(19) United States
(12) Patent Application Publication
(10) Pub. No.: US 2026/0273765 A1
(43) Pub. Date: Sep. 17, 2026

(54) ACTION CONTROL SYSTEM

(71) Applicant: SOFTBANK GROUP CORP., Minato-ku, Tokyo (JP)

(72) Inventor: Masayoshi SON, Minato-ku, Tokyo (JP)

(73) Assignee: SOFTBANK GROUP CORP., Minato-ku, Tokyo (JP)

(21) Appl. No.: 19/473,228

(22) PCT Filed: Apr. 9, 2024
(86) PCT No.: PCT/JP2024/014444

§ 371 Date: Oct. 7, 2025

(30) Foreign Application Priority Data
Apr. 11, 2023 (JP) 2023-064494
Apr. 12, 2023 (JP) 2023-065099
Apr. 13, 2023 (JP) 2023-065715
Apr. 14, 2023 (JP) 2023-066668
Apr. 17, 2023 (JP) 2023-067491
Apr. 18, 2023 (JP) 2023-068063
Apr. 18, 2023 (JP) 2023-068070
Apr. 19, 2023 (JP) 2023-068818
May 11, 2023 (JP) 2023-078688
May 11, 2023 (JP) 2023-078689
May 11, 2023 (JP) 2023-078692
May 12, 2023 (JP) 2023-079537
May 12, 2023 (JP) 2023-079538
May 12, 2023 (JP) 2023-079540
May 12, 2023 (JP) 2023-079541
May 12, 2023 (JP) 2023-079542
May 12, 2023 (JP) 2023-079543
May 12, 2023 (JP) 2023-079676
May 15, 2023 (JP) 2023-080318
May 15, 2023 (JP) 2023-080346
May 15, 2023 (JP) 2023-080389
May 16, 2023 (JP) 2023-080985
May 16, 2023 (JP) 2023-081011
May 16, 2023 (JP) 2023-081012
May 18, 2023 (JP) 2023-082447
May 18, 2023 (JP) 2023-082448
May 18, 2023 (JP) 2023-082572
May 19, 2023 (JP) 2023-083426
May 19, 2023 (JP) 2023-083453
May 19, 2023 (JP) 2023-083454
May 19, 2023 (JP) 2023-083455
May 19, 2023 (JP) 2023-083463
May 19, 2023 (JP) 2023-083516

(51) Int. Cl. B25J 11/00; B25J 13/00; B25J 13/08; B25J 19/00; B25J 19/02; G06V 40/16; G10L 25/63

(52) U.S. Cl. CPC B25J 11/001; B25J 13/003; B25J 13/087; B25J 19/0054; B25J 19/023; G06V 40/174; G10L 25/63

(57) ABSTRACT

An action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit reflects a detection result obtained by detecting a change in a body temperature of the user in generation of an answer of the interaction function, estimation of the emotion of the user, and estimation of the emotion of the robot.

### TECHNICAL FIELD

The present disclosure relates to an action control system.

### BACKGROUND ART

Patent Literature 1 discloses a technique for determining an appropriate action of a robot with respect to a state of a user. In the related art of Patent Literature 1, a reaction of a user in a case in which the robot executes a specific action is recognized, and in a case in which an action of the robot with respect to the recognized reaction of the user cannot be determined, the action of the robot is updated by receiving information regarding an action suitable for the recognized state of the user from a server.

### CITATION LIST

### Patent Literature

Patent Literature 1: Japanese Patent No. 6053847

### SUMMARY OF INVENTION

### Technical Problem

However, in the related art, there is room for improvement in causing a robot to execute an appropriate action for an action of the user.

### Solution to Problem

According to a first aspect of the disclosure, an action control system is provided. The action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit reflects a detection result obtained by detecting a change in a body temperature of the user in generation of an answer of the interaction function, estimation of the emotion of the user, and estimation of the emotion of the robot.

According to a second aspect of the disclosure, an action control system is provided. The action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit determines the action by reflecting a detection result obtained by detecting a change in a body temperature of the user in generation of an answer of the interaction function, estimation of the emotion of the user, and estimation of the emotion of the robot, and the action determined by the action determination unit includes an action of changing a surface temperature of at least a part of the robot.

According to a third aspect of the disclosure, an action control system is provided. The action control system includes an emotion determination unit that determines an emotion of a user or an emotion of a robot, and an action determination unit that generates action content of the robot with respect to an action of the user and the emotion of the user or an emotion of the robot on the basis of a sentence generation model having an interaction function of allowing the user and the robot to interact with each other, and determines an action of the robot corresponding to the action content, in which the action determination unit reflects a preference of the user extracted from a conversation of the user in generation of an answer of the interaction function, estimation of the emotion of the user, and estimation of the emotion of the robot.

According to a fourth aspect of the disclosure, an action control system is provided. The action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit reflects an estimated culture area of the user in generation of an answer of the interaction function, estimation of the emotion of the user, and estimation of the emotion of the robot.

According to a fifth aspect of the disclosure, an action control system is provided. The action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit collects characteristic information of the user and environment information at the time of acquiring the characteristic information, predicts interaction content of the user in a case in which the user starts an interaction with the robot on the basis of the collected characteristic information and environment information, and environment information at the time at which the user starts the interaction with the robot, and determines a speech having content including a result of the prediction as the action of the robot.

According to a sixth aspect of the disclosure, an action control system is provided. The action control system includes an emotion determination unit that determines an emotion of a user or an emotion of a robot, and an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit analyzes an SNS related to the user, and recognizes a thing in which the user is interested on the basis of a result of the analysis.

The action determination unit proposes a spot and/or an event recommended to the user at the current position of the user on the basis of the thing.

The action determination unit derives, among a plurality of spots and/or a plurality of events selected in advance, a route for going around the plurality of spots and/or the plurality of events according to at least a current congestion status, and determines action content of the robot to circulate the route.

The action determination unit provides guidance regarding at least one spot and/or event among the plurality of spots and/or the plurality of events in a predetermined language.

According to a seventh aspect of the disclosure, an action control system is provided. The action control system includes a user state recognition unit that recognizes a user state including an action of a user; an emotion determination unit that determines an emotion of the user or an emotion of a robot, and an action determination unit that determines an action of the robot corresponding to the user state and the emotion of the user or the emotion of the robot on the basis of a sentence generation model having an interaction function of allowing the user and the robot to interact with each other, in which the action determination unit reflects a detection result obtained by detecting at least one of contact of the user or a pressure change due to the contact in at least one of generation of an answer of the interaction function, estimation of the emotion of the user, or estimation of the emotion of the robot.

According to an eighth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include determining an action schedule of the electronic apparatus, and in a case in which it is determined to determine the action schedule of the electronic apparatus as the action of the electronic apparatus, the action determination unit determines a combination of an activation condition for activating the action schedule and content of the action schedule of the electronic apparatus, stores the combination in action schedule data, and determines to execute the content of the action schedule of the electronic apparatus in a case in which the activation condition for the action schedule data is satisfied.

The electronic apparatus may be a robot, and the action determination unit may determine any of a plurality of types of robot actions including no action as an action of the robot. Here, examples of the robot include a device that performs a physical operation, a device that outputs a video or a sound without performing a physical operation, and an agent that operates on software.

According to a ninth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, a surrounding environment of the user, the emotion of the user, or the emotion of the electronic apparatus, and an action determination model at a predetermined timing, any of a plurality of types of device operations including non-operation as an action of the electronic apparatus.

The electronic apparatus may be a robot, and the action determination unit may determine any of a plurality of types of robot actions including no action as an action of the robot. Here, examples of the robot include a device that performs a physical operation, a device that outputs a video or a sound without performing a physical operation, and an agent that operates on software.

According to a tenth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the action determination unit autonomously detects a body temperature of the user as a state of the user as the action of the electronic apparatus, and reflects the body temperature of the user in determination of the emotion of the user by the emotion determination unit on the basis of the body temperature of the user. Here, examples of the robot include a device that performs a physical operation, a device that outputs a video or a sound without performing a physical operation, and an agent that operates on software.

According to an eleventh aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include the electronic apparatus performing a speech or a gesture on the user, and the action determination unit autonomously detects the state of the user, and determines, in a case in which at least one of the emotion of the user or the emotion of the electronic apparatus is determined on the basis of the detected state of the user, content of the speech or the gesture according to at least one of the determined emotion of the user or the determined emotion of the electronic apparatus.

Here, examples of the robot include a device that performs a physical operation, a device that outputs a video or a sound without performing a physical operation, and an agent that operates on software.

According to a twelfth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include summarizing events of a previous day, and in a case in which summarizing the events of the previous day is determined as the action of the electronic apparatus, the action determination unit adds a fixed sentence for giving an instruction for summarizing the events of the previous day to text representing the history data, inputs the text to the action determination model to acquire a summary of the events of the previous day, and outputs the acquired summary by speech or gesture in a case in which a conversation of the user who remembers the events of the previous day or a gesture of the user who thinks of something is detected when the action determination unit is activated at a predetermined time on the next day or when the user wakes up.

Here, examples of the electronic apparatus include a device that performs a physical operation, a device that outputs a video or a sound without performing a physical operation, and an agent that operates on software.

According to a thirteenth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include autonomously changing a surface temperature of the electronic apparatus, and the action determination unit autonomously detects the state of the user as the action of the electronic apparatus, and determines, in a case in which at least one of the emotion of the user or the emotion of the electronic apparatus is determined on the basis of the detected state of the user, the surface temperature of the electronic apparatus according to at least one of the determined emotion of the user or the determined emotion of the electronic apparatus.

Here, examples of the robot include a device that performs a physical operation, a device that outputs a video or a sound without performing a physical operation, and an agent that operates on software.

According to a fourteenth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and a storage control unit that stores, in history data, event data including an emotion value determined by the emotion determination unit and data including the action of the user, in which the device operations include determining the emotion of the electronic apparatus in consideration of events of a previous day, and in a case in which it is determined to determine the emotion of the electronic apparatus in consideration of the events of the previous day as the action of the electronic apparatus, the action determination unit adds a fixed sentence for giving an instruction for summarizing the events of the previous day to text representing the history data, inputs the text to the action determination model to acquire a summary of the events of the previous day, adds a fixed sentence for inquiry about an emotion of the electronic apparatus on the next day to the text representing the history data, and inputs the text to the action determination model to determine the emotion of the electronic apparatus in consideration of the acquired summary.

Here, examples of the electronic apparatus include a device that performs a physical operation, a device that outputs a video or a sound without performing a physical operation, and an agent that operates on software.

According to a fifteenth aspect of the disclosure, an action control system is provided. The action control system includes
a state recognition unit that recognizes a user state including an action of a user and a state of an electronic apparatus,
an emotion determination unit that determines an emotion of the user or an emotion of the electronic apparatus,
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and
a storage control unit that stores, in history data, event data including an emotion value determined by the emotion determination unit and data including the action of the user, in which
the device operations include generating and reproducing music in consideration of events of a previous day, and
in a case in which it is determined to generate and reproduce music in consideration of the events of the previous day as the action of the electronic apparatus, the action determination unit acquires a summary of event data of the previous day stored in the history data, generates music based on the summary, and reproduces the generated music.

The electronic apparatus is a robot, and the action determination unit determines any of a plurality of types of robot actions including no action as an action of the robot.

Here, examples of the robot include a device that performs a physical operation, a device that outputs a video or a sound without performing a physical operation, and an agent that operates on software.

The action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the basis of an output of the sentence generation model.

The robot is mounted on a stuffed toy or is connected to a control target device mounted on the stuffed toy in a wireless or wired manner.

The robot is an agent for interacting with the user.

According to a sixteenth aspect of the disclosure, an action control system is provided. The action control system includes a state recognition unit that recognizes a user state including an action of a user and a state of an electronic apparatus,
an emotion determination unit that determines an emotion of the user or an emotion of the electronic apparatus, and an action determination unit that determines any of a plurality of types of device operations including non-operation as an action of the electronic apparatus by using at least one of the user state, the state of the electronic apparatus, the emotion of the user, or the emotion of the electronic apparatus, and an action determination model at a predetermined timing, in which
the action determination unit selects, according to an intensity of the emotion of the user or the emotion of the electronic apparatus determined by the emotion determination unit, one of action content of the electronic apparatus generated by using a sentence generation model having an interaction function as the action determination model or action content determined by using, as the action determination model, a reaction rule for determining an action of the electronic apparatus according to the action of the user and the emotion of the user or the emotion of the electronic apparatus.

The action determination unit selects the action content determined by using the reaction rule in a case in which an emotion value indicating the intensity of the emotion is a threshold or more, and selects the action content generated by using the sentence generation model in a case in which the emotion value is less than the threshold.

The electronic apparatus is a robot, and
the action determination unit determines any of a plurality of types of robot actions including no action as an action of the robot.

Here, examples of the robot include a device that performs a physical operation, a device that outputs a video or a sound without performing a physical operation, and an agent that operates on software.

In a case in which the action content is selected by using the sentence generation model, the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

The robot is mounted on a stuffed toy or is connected to a control target device mounted on the stuffed toy in a wireless or wired manner.

The robot is an agent for interacting with the user.

According to a seventeenth aspect of the disclosure, an action control system is provided. The action control system includes a state recognition unit that recognizes a user state including an action of a user and a state of an electronic apparatus,
an emotion determination unit that determines an emotion of the user or an emotion of the electronic apparatus, and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the action determination unit calculates a degree of coincidence between the action of the user, the emotion of the user, and/or the emotion of the electronic apparatus and a condition of a reaction rule for determining the action of the electronic apparatus according to the action of the user, the emotion of the user, and/or the emotion of the electronic apparatus, selects action content determined by using the reaction rule in a case in which the degree of coincidence is a threshold or more, and selects action content of the electronic apparatus determined by using a sentence generation model having an interaction function as the action determination model in a case in which the degree of coincidence is less than the threshold.

The electronic apparatus is a robot, and
the action determination unit determines any of a plurality of types of robot actions including no action as an action of the robot.

Here, examples of the robot include a device that performs a physical operation, a device that outputs a video or a sound without performing a physical operation, and an agent that operates on software.

In a case in which the action content is determined by using the sentence generation model, the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

The robot is mounted on a stuffed toy or is connected to a control target device mounted on the stuffed toy in a wireless or wired manner.

The robot is an agent for interacting with the user.

According to an eighteenth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include determining in advance a gesture of the electronic apparatus, and in a case in which it is determined to determine in advance a gesture of the electronic apparatus as the action of the electronic apparatus, the action determination unit determines an activation condition for activating the gesture and stores the activation condition in action schedule data, and determines to execute the gesture in a case in which the activation condition for the action schedule data is satisfied.

According to a first aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include determining in advance speech content of the electronic apparatus, and the action determination unit determines an activation condition for saying the speech content and stores the activation condition in action schedule data in a case in which it is determined to determine in advance speech content of the electronic apparatus as the action of the electronic apparatus, and determines to say the speech content in a case in which the activation condition for the action schedule data is satisfied.

The electronic apparatus may be a robot, and the action determination unit may determine any of a plurality of types of robot actions including no action as an action of the robot. Here, examples of the robot include a device that performs a physical operation, a device that outputs a video or a sound without performing a physical operation, and an agent that operates on software.

According to a nineteenth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the action determination unit reflects an estimated culture area of the user in at least one of output generation by the action determination model, determination of the emotion of the user by the emotion determination unit, or determination of the emotion of the electronic apparatus by the emotion determination unit.

Here, examples of the electronic apparatus include a device that performs a physical operation, a device that outputs a video or a sound without performing a physical operation, and an agent that operates on software.

According to a twentieth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus. The device operations include giving the user advice regarding a social networking service, and the action determination unit gives the user the advice regarding the social networking service in a case in which it is determined to give the user the advice regarding the social networking service as the action of the electronic apparatus.

Here, examples of the robot include a device that performs a physical operation, a device that outputs a video or a sound without performing a physical operation, and an agent that operates on software.

According to a twenty-first aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include giving health advice to the user, and the storage control unit stores, in the history data, a parameter representing a detected health condition of the user, and in a case in which it is determined to give health advice to the user as an action of the electronic apparatus, the action determination unit autonomously determines an action corresponding to the health condition of the user on the basis of the parameter representing the health condition of the user.

Here, examples of the robot include a device that performs a physical operation, a device that outputs a video or a sound without performing a physical operation, and an agent that operates on software.

According to a twenty-second aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include proposing to go to an art gallery, a museum, and an exhibition in accordance with a schedule of the user, and in a case in which it is determined to propose to go to an art gallery, a museum, or an exhibition as the action of the electronic apparatus, the action determination unit determines a proposed destination by using a sentence generation model on the basis of the event data stored in the history data.

Here, examples of the robot include a device that performs a physical operation, a device that outputs a video or a sound without performing a physical operation, and an agent that operates on software.

According to a twenty-third aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include reproducing user's favorite music, and in a case in which it is determined to reproduce user's favorite music as the action of the electronic apparatus, the action determination unit determines music to be reproduced on the basis of information regarding the user's taste in music stored in a storage unit.

The action determination unit determines the music to be reproduced on the basis of at least one of a taste in a type of music, a taste in a musical instrument, or a taste in a singer as the information regarding the user's taste in music.

The action determination unit determines a volume level according to the user's taste in a volume level.

The electronic apparatus is a robot, and the action determination unit determines any of a plurality of types of robot actions including no action as an action of the robot.

The action determination model is a sentence generation model having an interaction function, and the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

The robot is mounted on a stuffed toy or is connected to a control target device mounted on the stuffed toy in a wireless or wired manner.

The robot is an agent for interacting with the user.

Here, examples of the robot include a device that performs a physical operation, a device that outputs a video or a sound without performing a physical operation, and an agent that operates on software.

According to a twenty-fourth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include acquiring, as the event data, external data based on taste information of the user as the emotion of the user and outputting an image or a sound according to the event data, and in a case in which it is determined to output the event data based on the taste information of the user, the action determination unit outputs the determined event data through the device operations.

The electronic apparatus is a robot, and the action determination unit determines any of a plurality of types of robot actions including no action as an action of the robot.

The action determination model is a sentence generation model having an interaction function, and the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

The robot is mounted on a stuffed toy or is connected to a control target device mounted on the stuffed toy in a wireless or wired manner.

The robot is an agent for interacting with the user.

Here, examples of the robot include a device that performs a physical operation, a device that outputs a video or a sound without performing a physical operation, and an agent that operates on software.

According to a twenty-fifth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which the device operations include spontaneously and periodically detecting a state of the user, and the action determination unit spontaneously proposes an activity to the user in a case in which it is determined to propose the activity to the user as the action of the electronic apparatus.

Here, examples of the robot include a device that performs a physical operation, a device that outputs a video or a sound without performing a physical operation, and an agent that operates on software.

According to a twenty-sixth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include proposing an activity related to eating and drinking, and in a case in which it is determined to propose an activity related to eating and drinking as the action of the electronic apparatus, the action determination unit proposes the activity related to eating and drinking.

Here, examples of the robot include a device that performs a physical operation, a device that outputs a video or a sound without performing a physical operation, and an agent that operates on software.

According to a twenty-seventh aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include determining a schedule of the user, and in a case in which it is determined to propose a schedule as the action of the electronic apparatus, the action determination unit determines a schedule of the user to be proposed by using a sentence generation model on the basis of event data stored in the history data.

Here, examples of the robot include a device that performs a physical operation, a device that outputs a video or a sound without performing a physical operation, and an agent that operates on software.

According to a twenty-eighth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include autonomously converting a speech of the user into a question, and the action determination unit answers the question as the action of the electronic apparatus.

Here, examples of the robot include a device that performs a physical operation, a device that outputs a video or a sound without performing a physical operation, and an agent that operates on software.

According to a twenty-ninth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, and an action determination unit that determines, by using at least one of the user state, the state of the electronic apparatus, the emotion of the user, and the emotion of the electronic apparatus, and an action determination model at a predetermined timing, any of a plurality of types of device operations including non-operation as an action of the electronic apparatus, in which the device operations include increasing a vocabulary and speaking the increased vocabulary, and in a case in which it is determined to increase a vocabulary as the action of the electronic apparatus, the action determination unit increases the vocabulary, and in a case in which it is determined to speak the increased vocabulary, the action determination unit speaks the increased vocabulary.

Here, examples of the robot include a device that performs a physical operation, a device that outputs a video or a sound without performing a physical operation, and an agent that operates on software.

According to a thirtieth aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include asking the user a question about an important action performed by the user in the past, and the action determination unit stores, as the action of the electronic apparatus, the action of the user together with an emotion value of the user, stores the action as the important action in a case in which the emotion value of the user exceeds a predetermined value, and determines to ask a question about the past important action in a case in which the action of the user at a different timing coincides with the important action.

Here, examples of the robot include a device that performs a physical operation, a device that outputs a video or a sound without performing a physical operation, and an agent that operates on software.

According to a thirty-first aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, characteristic information including a characteristic of the user, and situation information at the time of acquiring the characteristic information, in which the device operations include speaking to the user, in a case in which it is determined to speak to the user as the action of the electronic apparatus, the action determination unit estimates interaction content of the user with the electronic apparatus on the basis of the history data and situation information at the time of speaking to the user, and determines speech content to the user on the basis of a result of the estimation. Here, examples of the electronic apparatus include a device that performs a physical operation, a device that outputs a video or a sound without performing a physical operation, and an agent that operates on software.

According to the thirty-first aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, characteristic information including a characteristic of the user, and situation information at the time of acquiring the characteristic information, in which the device operations include reproducing specific music data, and in a case in which it is determined to reproduce the specific music data as the action of the electronic apparatus, the action determination unit determines the specific music data to be reproduced on the basis of the history data and situation information at the time of reproducing the specific music data.

According to a thirty-second aspect of the disclosure, an action control system is provided. The action control system includes a state recognition unit that recognizes a user state including an action of a user and a state of an electronic apparatus, an emotion determination unit that determines an emotion of the user or an emotion of the electronic apparatus, an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, and a storage control unit that stores, in history data, event data including an emotion value determined by the emotion determination unit and data including the action of the user, in which the device operations include setting action content of selecting at least one of two or more things and proposing the thing to the user, and in a case in which the action determination unit spontaneously or periodically detects a state of the user, and determines to propose at least one thing from among two or more things as the action of the electronic apparatus on the basis of at least one of the detected state of the user, history data regarding the user, or information preferred by the user, the action determination unit executes the action content.

Here, examples of the robot include a device that performs a physical operation, a device that outputs a video or a sound without performing a physical operation, and an agent that operates on software.

### BRIEF DESCRIPTION OF DRAWINGS

FIG. 1 schematically illustrates an example of a system 5 according to the present embodiment.

FIG. 2 schematically illustrates a functional configuration of a robot 100.

FIG. 3 schematically illustrates an example of an operation flow in the robot 100.

FIG. 4 schematically illustrates an example of a hardware configuration of a computer 1200.

FIG. 5 illustrates an emotion map 400 on which a plurality of emotions are mapped.

FIG. 6 illustrates an emotion map 900 on which a plurality of emotions are mapped.

FIG. 7(A) is an external view of a stuffed toy according to another embodiment, and FIG. 7(B) is an internal structural view of the stuffed toy.

FIG. 8 is a rear front view of a stuffed toy according to another embodiment.

FIG. 9 schematically illustrates a functional configuration of a robot 100 according to a second embodiment.

FIG. 10 schematically illustrates an example of an operation flow of collection processing in the robot 100 according to the second embodiment.

FIG. 11 schematically illustrates an example of an operation flow of response processing in the robot 100 according to the second embodiment.

FIG. 12 schematically illustrates an example of an operation flow of autonomous processing of the robot 100 according to the second embodiment.

FIG. 13 schematically illustrates a functional configuration of a stuffed toy 100N according to a third embodiment.

FIG. 14 schematically illustrates a functional configuration of an agent system 500 according to a fourth embodiment.

FIG. 15 illustrates an example of an operation of the agent system.

FIG. 16 illustrates an example of an operation of the agent system.

FIG. 17 schematically illustrates a functional configuration of an agent system 700 of smart glasses according to a fifth embodiment.

FIG. 18 illustrates an example of a usage mode of an agent system using smart glasses.

### DESCRIPTION OF EMBODIMENTS

Hereinafter, the disclosure will be described through embodiments of the invention, but the following embodiments do not limit the invention according to the claims. Not all combinations of features described in the embodiments are essential to the solution of the invention.

### First Embodiment

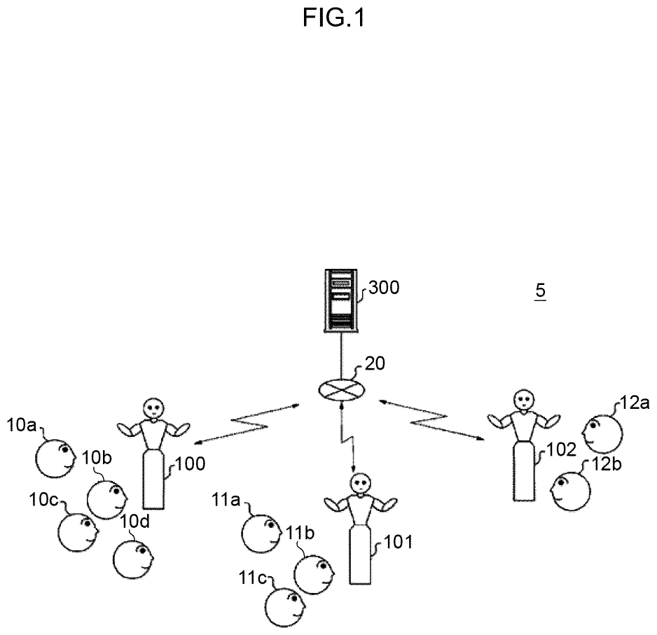

FIG. 1 schematically illustrates an example of a system 5 according to the present embodiment. The system 5 includes a robot 100, a robot 101, a robot 102, and a server 300. A user 10a, a user 10b, a user 10c, and a user 10d are users of the robot 100. The user 11a, the user 11b, and the user 11c are users of the robot 101. A user 12a and a user 12b are users of the robot 102. In the description of the present embodiment, the user 10a, the user 10b, the user 10c, and the user 10d may be collectively referred to as a user 10. The user 11a, the user 11b, and the user 11c may be collectively referred to as a user 11. The user 12a and the user 12b may be collectively referred to as a user 12. The robot 101 and the robot 102 have substantially the same functions as those of the robot 100. Therefore, the system 5 will be described focusing on the functions of the robot 100.

The robot 100 has a conversation with the user 10 and provides a video to the user 10. In this case, the robot 100 has a conversation with the user 10, provides a video to the user 10, and the like in cooperation with the server 300 and the like that can communicate via a communication network 20. For example, the robot 100 not only learns an appropriate conversation by itself, but also performs learning so that a conversation with the user 10 can be advanced more appropriately in cooperation with the server 300. The robot 100 causes the server 300 to record captured video data and the like of the user 10, requests the server 300 to transmit the video data and the like as necessary, and provides the video data and the like to the user 10.

The robot 100 has an emotion value indicating the type of its own emotion. For example, the robot 100 has emotion values indicating the intensities of respective emotions such as “happy”, “angry”, “sad”, “pleasant”, “pleased”, “displeased”, “safe”, “anxious”, “gloomy”, “excited”, “worried”, “relieved”, “sense of fulfilment”, “sense of emptiness”, and “normal”. For example, in a case in which the robot 100 has a conversation with the user 10 in a state in which the emotion value of excitement is great, the robot emits a voice at a fast speed. As described above, the robot 100 can express its own emotion by action.

The robot 100 may be configured to determine an action of the robot 100 corresponding to the emotion of the user 10 by matching a sentence generation model using artificial intelligence (AI) with an emotion engine. Specifically, the robot 100 may be configured to recognize an action of the user 10, determine an emotion of the user 10 for the action of the user, and determine an action of the robot 100 corresponding to the determined emotion.

More specifically, in a case in which the robot 100 recognizes the action of the user 10, the robot 100 automatically generates the action content to be taken by the robot with respect to the action of the user 10 by using the preset sentence generation model. The sentence generation model may be interpreted as an algorithm and an operation for automatic interaction processing using text. Since the sentence generation model is known as disclosed in, for example, Japanese Patent Application Laid-Open (JP-A) No. 2018 081444 and chatGPT (Internet search <URL: https://openai.com/blog/chatgpt>), a detailed description thereof will be omitted. Such a sentence generation model is configured by a large language model (LLM).

As described above, in the present embodiment, it is possible to reflect the emotions of the user 10 and the robot 100 and various types of linguistic information in the action of the robot 100 by combining the large language model and the emotion engine. That is, according to the present embodiment, a synergistic effect can be obtained by combining the sentence generation model and the emotion engine.

The robot 100 has a function of recognizing an action of the user 10. The robot 100 recognizes the action of the user 10 by analyzing a face image of the user 10 acquired by a camera function and the voice of the user 10 acquired by a microphone function. The robot 100 determines an action to be executed by the robot 100 on the basis of the recognized action of the user 10 or the like.

The robot 100 stores a rule defining an action to be executed by the robot 100 on the basis of the emotion of the user 10, the emotion of the robot 100, and the action of the user 10, and performs various actions according to the rule.

Specifically, the robot 100 has a reaction rule for determining an action of the robot 100 on the basis of the emotion of the user 10, the emotion of the robot 100, and the action of the user 10. In the reaction rule, for example, in a case in which the action of the user 10 is “smiling”, an action of “smiling” is set as the action of the robot 100. In the reaction rule, an action of “apologizing” is defined as an action of the robot 100, with respect to a case in which the action of the user 10 is “angry”. In the reaction rule, an action of “answering” is defined as an action of the robot 100 with respect to a case in which the action of the user 10 is “inquiry”. In the reaction rule, an action of “talking” is defined as an action of the robot 100 with respect to a case in which the action of the user 10 is “gloomy”.

In a case in which the robot 100 recognizes that the action of the user 10 is “angry” on the basis of the reaction rule, the robot selects an action of “apologizing” defined in the reaction rule as the action to be executed by the robot 100. For example, in the case of selecting the action of “apologizing”, the robot 100 performs an action of “apologizing” and outputs a voice expressing the word “apologizing”.

In a case in which a condition that the emotion of the robot 100 is “normal” (that is, “happy”=0, “angry”=0, “sad”=0, and “pleasant”=0) and the state of the user 10 is “one person, lonely” is satisfied, it is defined that the change content of the emotion that the emotion of the robot 100 is “worried” and the action of “talking” can be executed.

In a case in which the robot 100 recognizes that the current emotion of the robot 100 is “normal” and the user 10 is alone in a lonely state on the basis of the reaction rule, the emotion value of “sad” of the robot 100 is increased. The robot 100 selects the action of “talking” defined in the reaction rule as an action to be executed on the user 10. For example, in a case in which the action of “talking” is selected, the robot 100 converts a word “What's wrong?” indicating that the robot is concerned into a voice indicating that the robot is concerned, and outputs the voice.

The robot 100 transmits, to the server 300, user reaction information indicating that a positive reaction has been obtained from the user 10 through this action. The user reaction information includes, for example, the user action of “angry”, the action of the robot 100 of “apologizing”, the positive reaction of the user 10, and an attribute of the user 10.

The server 300 stores the user reaction information received from the robot 100. The server 300 receives and stores the user reaction information not only from the robot 100 but also from each of the robot 101 and the robot 102. The server 300 analyzes the user reaction information from the robot 100, the robot 101, and the robot 102, and updates the reaction rule.

The robot 100 receives the updated reaction rule from the server 300 by inquiring the server 300 about the updated reaction rule. The robot 100 incorporates the updated reaction rule into the reaction rule stored in the robot 100. As a result, the robot 100 can incorporate the reaction rule acquired by the robot 101, the robot 102, or the like into its own reaction rule.

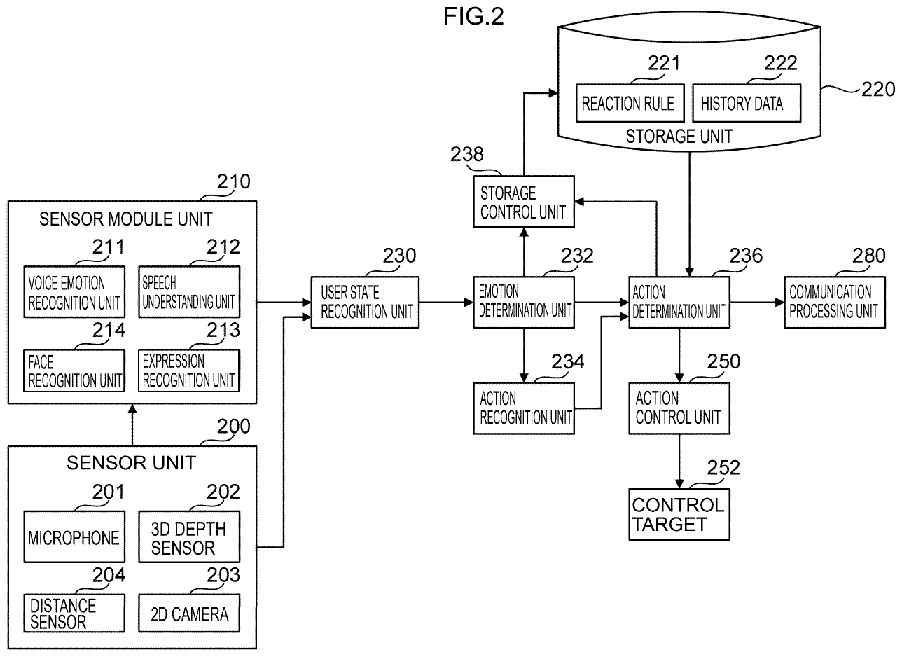

FIG. 2 schematically illustrates a functional configuration of the robot 100. The robot 100 includes a sensor unit 200, a sensor module unit 210, a storage unit 220, a user state recognition unit 230, an emotion determination unit 232, an action recognition unit 234, an action determination unit 236, a storage control unit 238, an action control unit 250, a control target 252, and a communication processing unit 280.

Examples of the control target 252 include a display device, a speaker, an LED of an eye portion, and motors that drive an arm, a hand, a foot, and the like. The posture and the action of the robot 100 are controlled by controlling motors for arms, hands, and feet. Some of the emotions of the robot 100 can be expressed by controlling these motors. The expression of the robot 100 can be expressed by controlling a light emission state of the LED of the eye portion of the robot 100. The posture, the gesture, and the expression of the robot 100 are examples of the attitude of the robot 100.

The sensor unit 200 includes a microphone 201, a 3D depth sensor 202, a 2D camera 203, and a distance sensor 204. The microphone 201 continuously detects a voice and outputs voice data. The microphone 201 may be provided on the head of the robot 100 and may have a function of performing binaural recording. The 3D depth sensor 202 analyzes infrared patterns from infrared images continuously captured by an infrared camera by continuously applying the infrared patterns, and detects an outline of an object. The 2D camera 203 is an example of an image sensor. The 2D camera 203 captures an image with visible light and generates image information of visible light. The distance sensor 204 detects a distance to an object by emitting, for example, laser light or ultrasonic waves. The sensor unit 200 may further include a clock, a gyro sensor, a touch sensor, a sensor for motor feedback, and the like.

Among the constituents of the robot 100 illustrated in FIG. 2, the constituents other than the control target 252 and the sensor unit 200 are examples of the constituents included in the action control system included in the robot 100. The action control system of the robot 100 controls the control target 252.

The storage unit 220 includes a reaction rule 221 and history data 222. The history data 222 includes past emotion values and a history of actions of the user 10. The emotion values and the history of actions are recorded for each user 10 by being associated with identification information of the user 10, for example. At least a part of the storage unit 220 is implemented by a storage medium such as a memory. A person DB that stores a face image of the user 10, attribute information of the user 10, and the like may be included. Among the constituents of the robot 100 illustrated in FIG. 2, the functions of the constituents other than the control target 252, the sensor unit 200, and the storage unit 220 can be realized by a CPU operating on the basis of a program. For example, the functions of these constituents can be implemented as the operation of the CPU by basic software (OS) and a program operating on the OS.

The sensor module unit 210 includes a voice emotion recognition unit 211, a speech understanding unit 212, an expression recognition unit 213, and a face recognition unit 214. Information detected by the sensor unit 200 is input to the sensor module unit 210. The sensor module unit 210 analyzes information detected by the sensor unit 200 and outputs an analysis result to the user state recognition unit 230.

The voice emotion recognition unit 211 of the sensor module unit 210 analyzes the voice of the user 10 detected by the microphone 201 to recognize the emotion of the user 10. For example, the voice emotion recognition unit 211 extracts a feature amount such as a frequency component of the voice and recognizes the emotion of the user 10 on the basis of the extracted feature amount. The speech understanding unit 212 analyzes the voice of the user 10 detected by the microphone 201 and outputs text information indicating the speech content of the user 10.

The expression recognition unit 213 recognizes the expression of the user 10 and the emotion of the user 10 from the image of the user 10 captured by the 2D camera 203. For example, the expression recognition unit 213 recognizes the expression and the emotion of the user 10 on the basis of the shapes, positional relationships, and the like of the eyes and the mouth.

The face recognition unit 214 recognizes the face of the user 10. The face recognition unit 214 recognizes the user 10 by matching a face image stored in a person DB (not illustrated) with a face image of the user 10 captured by the 2D camera 203.

The user state recognition unit 230 recognizes a state of the user 10 on the basis of the information analyzed by the sensor module unit 210. For example, processing mainly related to perception is performed by using the analysis result from the sensor module unit 210. For example, perception information such as “There is one father.” and “The probability that the father does not smile is 90%.” is generated. Processing of understanding the meaning of the generated perception information is performed. For example, semantic information such as “One father looks lonely.” is generated.

The emotion determination unit 232 determines an emotion value indicating the emotion of the user 10 on the basis of the information analyzed by the sensor module unit 210 and the state of the user 10 recognized by the user state recognition unit 230. For example, the information analyzed by the sensor module unit 210 and the recognized state of the user 10 are input to a neural network trained in advance, and an emotion value indicating the emotion of the user 10 is acquired.

Here, the emotion value indicating the emotion of the user 10 is a value indicating whether the emotion of the user is positive or negative. For example, in a case in which the emotion of the user is a bright emotion accompanied with pleasure or comfort, such as “happy”, “pleasant”, “pleased”, “safe”, “excited”, “relieved”, and “sense of fulfillment”, a positive value is indicated, and the value becomes larger as the emotion becomes brighter. In a case in which the emotion of the user is an emotion that makes the user feel unpleasant, such as “angry”, “sad”, “displeased”, “anxious”, “gloomy”, “worried”, and “sense of emptiness”, a negative value is indicated, and an absolute value of the negative value increases as the user feels unpleasant. In a case in which the emotion of the user is not included in any of the above emotions (“normal”), a value of 0 is indicated.

The emotion determination unit 232 determines an emotion value indicating the emotion of the robot 100 on the basis of the information analyzed by the sensor module unit 210 and the state of the user 10 recognized by the user state recognition unit 230.

The emotion value of the robot 100 includes an emotion value for each of the plurality of emotion classifications, and is, for example, a value (0 to 5) indicating the intensity of each of “happy”, “angry”, “sad”, and “pleasant”.

Specifically, the emotion determination unit 232 determines an emotion value indicating the emotion of the robot 100 according to a rule for updating the emotion value of the robot 100, the rule being defined in association with the information analyzed by the sensor module unit 210 and the state of the user 10 recognized by the user state recognition unit 230.

For example, in a case in which the user state recognition unit 230 recognizes that the user 10 looks lonely, the emotion determination unit 232 increases the emotion value of “sad” of the robot 100. In a case in which the user state recognition unit 230 recognizes that the user 10 has a smiling face, the emotion value of “happy” of the robot 100 is increased.

The emotion determination unit 232 may determine the emotion value indicating the emotion of the robot 100 in further consideration of the state of the robot 100. For example, in a case in which a remaining battery level of the robot 100 is low or in a case in which the surrounding environment of the robot 100 is dark, the emotion value of “sad” of the robot 100 may be increased. In the case of the user 10 continuously talking to the robot even though the remaining battery level is low, the emotion value of “angry” may be increased.

The action recognition unit 234 recognizes an action of the user 10 on the basis of the information analyzed by the sensor module unit 210 and the state of the user 10 recognized by the user state recognition unit 230. For example, the information analyzed by the sensor module unit 210 and the recognized state of the user 10 are input to a neural network trained in advance, probabilities of a plurality of predetermined action classifications (for example, “smile”, “angry”, “ask a question”, and “gloomy”) are acquired, and an action classification having the highest probability is recognized as the action of the user 10.

As described above, in the present embodiment, the robot 100 acquires the speech content of the user 10 after identifying the user 10, but in acquiring and using the speech content, the action control system of the robot 100 according to the present embodiment considers protection of personal information and privacy of the user 10 in addition to acquiring necessary consent according to laws and regulations from the user 10.

The action determination unit 236 determines an action corresponding to the action of the user 10 recognized by the action recognition unit 234 on the basis of the current emotion value of the user 10 determined by the emotion determination unit 232, the history data 222 of the past emotion values determined by the emotion determination unit 232 before the current emotion value of the user 10 is determined, and the emotion value of the robot 100. In the present embodiment, a case in which the action determination unit 236 uses one most recent emotion value included in the history data 222 as the past emotion value of the user 10 will be described, but the disclosed technology is not limited to this aspect. For example, the action determination unit 236 may use a plurality of most recent emotion values as the past emotion values of the user 10, or may use emotion values that are earlier by a unit period such as one day before. The action determination unit 236 may determine an action corresponding to the action of the user 10 in further consideration of the history of the past emotion values of the robot 100 in addition to the current emotion value of the robot 100. The action determined by the action determination unit 236 includes a gesture performed by the robot 100 or speech content of the robot 100.

The action determination unit 236 according to the present embodiment determines the action of the robot 100 on the basis of a combination of the past emotion value and the current emotion value of the user 10, the emotion value of the robot 100, the action of the user 10, and the reaction rule 221, as an action corresponding to the action of the user 10. For example, in a case in which the past emotion value of the user 10 is a positive value and the current emotion value is a negative value, the action determination unit 236 determines an action for positively changing the emotion value of the user 10 as an action corresponding to the action of the user 10.

The reaction rule 221 defines an action of the robot 100 according to the combination of the past emotion value and the current emotion value of the user 10, the emotion value of the robot 100, and the action of the user 10. For example, in a case in which the past emotion value of the user 10 is a positive value, the current emotion value is a negative value, and the action of the user 10 is gloomy, a combination of a gesture and speech content at the time of making an inquiry to encourage the user 10 including a gesture is defined as the action of the robot 100.

For example, the reaction rule 221 defines an action of the robot 100 for all combinations of patterns of emotion values of the robot 100 (1296 patterns that is the fourth power of six values of the values “0” to “5” of “happy”, “angry”, “sad”, and “pleasant”), a pattern of the combination of the past emotion value and the current emotion value of the user 10, and an action pattern of the user 10. That is, for each pattern of the emotion value of the robot 100, the action of the robot 100 according to the action pattern of the user 10 is defined for each of a plurality of combinations such as combinations of the past emotion value and the current emotion value of the user 10 being a negative value and a negative value, a negative value and a positive value, a positive value and a negative value, a positive value and a positive value, a negative value and normal, and normal and normal. The action determination unit 236 may transition to an operation mode of determining the action of the robot 100 by using the history data 222, for example, in a case in which the user 10 has made a speech that intends a conversation continued from a past topic such as “I want to talk about the topic I discussed earlier”.

In the reaction rule 221, at least one of a gesture and statement content may be defined as an action of the robot 100 for each pattern (1296 patterns) of the emotion values of the robot 100 at the maximum. Alternatively, in the reaction rule 221, at least one of a gesture and statement content may be defined as an action of the robot 100 for each of groups of the patterns of the emotion values of the robot 100.

The intensity of each gesture included in the action of the robot 100 defined in the reaction rule 221 is defined in advance. In each piece of speech content included in the action of the robot 100 defined in the reaction rule 221, the intensity of the speech content is defined in advance.

The storage control unit 238 determines whether or not to store data including the action of the user 10 in the history data 222 on the basis of the intensity of the action predefined for the action determined by the action determination unit 236 and the emotion value of the robot 100 determined by the emotion determination unit 232.

Specifically, in a case in which a total value of the intensities that is a sum of a sum total of the emotion values for the plurality of respective emotion classifications of the robot 100, the intensity predefined for the gesture included in the action determined by the action determination unit 236, and the intensity predefined for the speech content included in the action determined by the action determination unit 236 is a threshold or more, it is determined to store data including the action of the user 10 in the history data 222.

In a case in which the storage control unit 238 determines to store the data including the action of the user 10 in the history data 222, the action determined by the action determination unit 236, information (for example, any surrounding information including data such as a voice, an image, and a smell at the place) analyzed by the sensor module unit 210 from the current time point to a certain period before, and a state (for example, the expression and the emotion of the user 10) of the user 10 recognized by the user state recognition unit 230 are stored in the history data 222.

The action control unit 250 controls the control target 252 on the basis of the action determined by the action determination unit 236. For example, in a case in which the action determination unit 236 determines an action including a speech, the action control unit 250 causes a speaker included in the control target 252 to output a voice. In this case, the action control unit 250 may determine an emission speed of the voice on the basis of the emotion value of the robot 100. For example, the action control unit 250 determines a higher voice emission speed as the emotion value of the robot 100 becomes greater. As described above, the action control unit 250 determines an execution form of the action determined by the action determination unit 236 on the basis of the emotion value determined by the emotion determination unit 232.

The action control unit 250 may recognize a change in emotion of the user 10 with respect to execution of the action determined by the action determination unit 236. For example, the change in emotion may be recognized on the basis of the voice or expression of the user 10. A change in the emotion of the user 10 may be recognized on the basis of detection of an impact by the touch sensor included in the sensor unit 200. In a case in which an impact is detected by the touch sensor included in the sensor unit 200, it may be recognized that the emotion of the user 10 is worsened, or in a case in which it is determined that the reaction of the user 10 is smiling or happy from the detection result in the touch sensor included in the sensor unit 200, it may be recognized that the emotion of the user 10 is improved. Information indicating the reaction of the user 10 is output to the communication processing unit 280.

After the action control unit 250 executes the action determined by the action determination unit 236 in the execution form determined according to the emotion of the robot 100, the emotion determination unit 232 further changes the emotion value of the robot 100 on the basis of the user's reaction to the execution of the action. Specifically, the emotion determination unit 232 increases the emotion value of “happy” of the robot 100 in a case in which the user's reaction to the action determined by the action determination unit 236 performed on the user in the execution form determined by the action control unit 250 is not poor, and the emotion determination unit 232 increases the emotion value of “sad” of the robot 100 in a case in which the user's reaction to the action determined by the action determination unit 236 performed on the user in the execution form determined by the action control unit 250 is poor.

The action control unit 250 expresses the emotion of the robot 100 on the basis of the determined emotion value of the robot 100. For example, in a case in which the emotion value of “happy” of the robot 100 is increased, the action control unit 250 controls the control target 252 to cause the robot 100 to perform a gesture of being happy. In a case in which the emotion value of “sad” of the robot 100 is increased, the action control unit 250 controls the control target 252 so that the posture of the robot 100 becomes a head-drooping posture.

The communication processing unit 280 performs communication with the server 300. As described above, the communication processing unit 280 transmits the user reaction information to the server 300. The communication processing unit 280 receives the updated reaction rule from the server 300. Upon receiving the updated reaction rule from the server 300, the communication processing unit 280 updates the reaction rule 221.

The server 300 performs communication between the robot 100, the robot 101, and the robot 102 and the server 300, receives the user reaction information transmitted from the robot 100, and updates the reaction rule on the basis of the reaction rule including the action for which a positive reaction has been obtained.

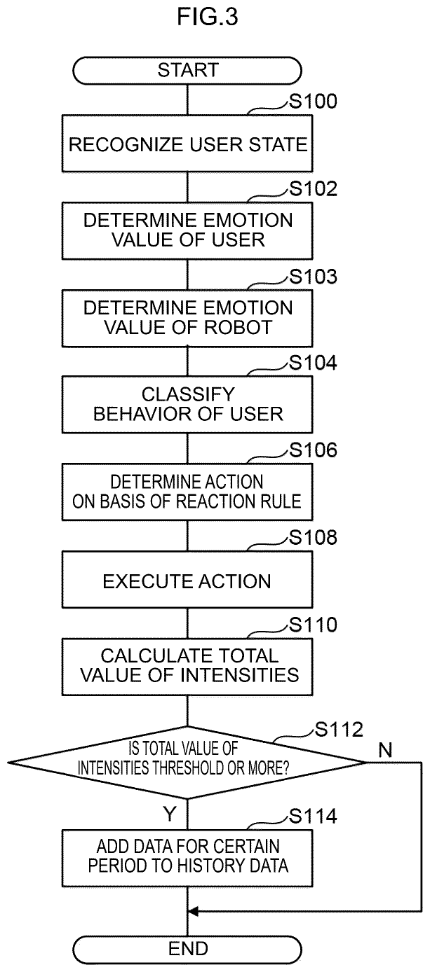

FIG. 3 schematically illustrates an example of an operation flow related to an operation of determining an action of the robot 100. The operation flow illustrated in FIG. 3 is repeatedly executed. In this case, it is assumed that information analyzed by the sensor module unit 210 is input. “S” in the operation flow represents a step to be executed.

First, in step S100, the user state recognition unit 230 recognizes a state of the user 10 on the basis of the information analyzed by the sensor module unit 210.

In step S102, the emotion determination unit 232 determines an emotion value indicating the emotion of the user 10 on the basis of the information analyzed by the sensor module unit 210 and the state of the user 10 recognized by the user state recognition unit 230.

In step S103, the emotion determination unit 232 determines an emotion value indicating the emotion of the robot 100 on the basis of the information analyzed by the sensor module unit 210 and the state of the user 10 recognized by the user state recognition unit 230. The emotion determination unit 232 adds the determined emotion value of the user 10 to the history data 222.

In step S104, the action recognition unit 234 recognizes an action classification of the user 10 on the basis of the information analyzed by the sensor module unit 210 and the state of the user 10 recognized by the user state recognition unit 230.

In step S106, the action determination unit 236 determines an action of the robot 100 on the basis of a combination of the current emotion value of the user 10 determined in step S102 and the past emotion value included in the history data 222, the emotion value of the robot 100, the action of the user 10 recognized by the action recognition unit 234, and the reaction rule 221.

In step S108, the action control unit 250 controls the control target 252 on the basis of the action determined by the action determination unit 236.

In step S110, the storage control unit 238 calculates a total value of the intensities on the basis of the intensity of the action predefined for the action determined by the action determination unit 236 and the emotion value of the robot 100 determined by the emotion determination unit 232.

In step S112, the storage control unit 238 determines whether or not the total value of the intensities is a threshold or more. In a case in which the total value of the intensities is less than the threshold, the data including the action of the user 10 is not stored in the history data 222, and the processing is ended. On the other hand, in a case in which the total value of the intensities is the threshold or more, the processing proceeds to step S114.

In step S114, the action determined by the action determination unit 236, the information analyzed by the sensor module unit 210 from the current time point to a certain period before, and the state of the user 10 recognized by the user state recognition unit 230 are stored in the history data 222.

As described above, the robot 100 determines the emotion value indicating the emotion of the robot 100 on the basis of the user state, and determines whether or not to store data including the action of the user 10 in the history data 222 on the basis of the emotion value of the robot 100. As a result, the capacity of the history data 222 that stores data including an action of the user 10 can be suppressed. For example, in a case in which the robot 100 determines that the user state of 10 years ago is the same as the user state after 10 years, the robot 100 reads the history data 222 of 10 years ago, and thus, can present the state of the user 10 of 10 years ago (for example, an expression and an emotion of the user 10), and further, any surrounding information including data such as a voice, an image, and a smell at the place to the user 10.

According to the robot 100, it is possible to cause the robot 100 to execute an appropriate action with respect to the action of the user 10. Conventionally, an action of a user is classified to determine an action including an expression or an appearance of a robot. On the other hand, the robot 100 determines the current emotion value of the user 10, and executes an action on the user 10 on the basis of the past emotion value and the current emotion value. Therefore, for example, in a case in which the user 10 who was fine yesterday is depressed today, the robot 100 may say, “You were fine yesterday. What's wrong with you today?”. The robot 100 may also make a speech with a gesture. For example, in a case in which the user 10 who was depressed yesterday is fine today, the robot 100 may say, “You were depressed yesterday, but you look fine today?”. For example, in a case in which the user 10 who was fine yesterday is better today than yesterday, the robot 100 may say, “You are better today than yesterday. What's better than yesterday?”. For example, the robot 100 may make a speech such as “Recently, the mood is stable, which is good.” to the user 10 whose emotion value is 0 or more and whose state in which the fluctuation range of the emotion value is within a certain range continues.

For example, in a case in which the robot 100 asks a question of “Did you finish the homework that you said yesterday?” to the user 10 and an answer of “I did it” is obtained from the user 10, the robot may make an affirmative speech such as “Good!” and make an affirmative gesture such as applause or thumbs-up. For example, in a case in which the user 10 says, “The presentation we discussed the day before yesterday was successful”, the robot 100 can make an affirmative speech such as “Good job!” and also make the above affirmative gesture. As described above, the robot 100 performs an action based on the history of the state of the user 10, whereby the user 10 can be expected to feel a sense of closeness to the robot 100.

In the above embodiment, the case in which the robot 100 recognizes the user 10 by using the face image of the user 10 has been described, but the disclosed technology is not limited to this aspect. For example, the robot 100 may recognize the user 10 by using a voice uttered by the user 10, a mail address of the user 10, an ID of an SNS of the user 10, an ID card in which a wireless IC tag is built and which is possessed by the user 10, or the like.

The robot 100 is an example of an electronic apparatus including an action control system. An application target of the action control system is not limited to the robot 100, and the action control system can be applied to various electronic apparatuses. The functions of the server 300 may be implemented by one or more computers. At least some functions of the server 300 may be implemented by a virtual machine. At least some of the functions of the server 300 may be implemented in a cloud.

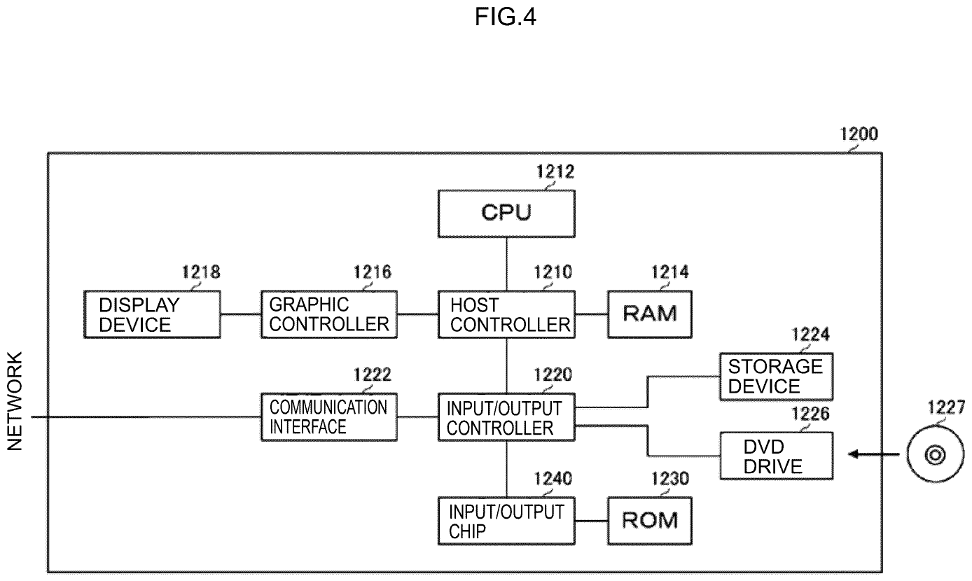

FIG. 4 schematically illustrates an example of a hardware configuration of a computer 1200 that functions as the robot 100 and the server 300. A program installed in the computer 1200 can cause the computer 1200 to function as one or more “units” of the device according to the present embodiment, or cause the computer 1200 to execute an operation associated with the device according to the present embodiment or one or more “units” thereof, and/or cause the computer 1200 to execute a process according to the present embodiment or a stage of the process. Such a program may be executed by a CPU 1212 to cause the computer 1200 to execute certain operations associated with some or all of the blocks in the flowcharts and block diagrams described herein.

The computer 1200 according to the present embodiment includes a CPU 1212, a RAM 1214, and a graphic controller 1216, which are connected to each other via a host controller 1210. The computer 1200 also includes input/output units such as a communication interface 1222, a storage device 1224, a DVD drive 1226, and an IC card drive, which are connected to the host controller 1210 via an input/output controller 1220. The DVD drive 1226 may be a DVD-ROM drive, a DVD-RAM drive, or the like. The storage device 1224 may be a hard disk drive, a solid state drive, or the like. The computer 1200 also includes a ROM 1230 and legacy input/output units such as a keyboard, which are connected to the input/output controller 1220 via an input/output chip 1240.

The CPU 1212 operates according to programs stored in the ROM 1230 and the RAM 1214, thereby controlling each unit. The graphic controller 1216 acquires image data generated by the CPU 1212 in a frame buffer or the like provided in the RAM 1214 or itself, and causes the image data to be displayed on the display device 1218.

The communication interface 1222 communicates with other electronic devices via a network. The storage device 1224 stores programs and data used by the CPU 1212 in the computer 1200. The DVD drive 1226 reads a program or data from the DVD-ROM 1227 or the like and provides the program or data to the storage device 1224. The IC card drive reads a program and data from the IC card and/or writes the program and data to the IC card.

The ROM 1230 stores therein a boot program or the like executed by the computer 1200 at the time of activation and/or a program depending on hardware of the computer 1200. The input/output chip 1240 may also connect various input/output units to the input/output controller 1220 via a USB port, a parallel port, a serial port, a keyboard port, a mouse port, or the like.

The program is provided by a computer readable storage medium such as the DVD-ROM 1227 or an IC card. The program is read from a computer readable storage medium, installed in the storage device 1224, the RAM 1214, or the ROM 1230, which is also an example of a computer readable storage medium, and executed by the CPU 1212. The information processing described in such a program is read by the computer 1200 and provides cooperation between the program and the various types of hardware resources. A device or a method may be configured by implementing an operation or processing of information according to use of the computer 1200.

For example, in a case in which communication is executed between the computer 1200 and an external device, the CPU 1212 may execute a communication program loaded in the RAM 1214 and instruct the communication interface 1222 to perform communication processing on the basis of processing described in the communication program. Under the control of the CPU 1212, the communication interface 1222 reads transmission data stored in a transmission buffer area provided in a recording medium such as the RAM 1214, the storage device 1224, the DVD-ROM 1227, or the IC card, transmits the read transmission data to the network, or writes reception data received from the network to a reception buffer area or the like provided on the recording medium.

The CPU 1212 may cause the RAM 1214 to read all or a necessary portion of a file or database stored in an external recording medium such as the storage device 1224, the DVD drive 1226 (DVD-ROM 1227), or the IC card, and may execute various types of processing on data in the RAM 1214. Next, the CPU 1212 may write back the processed data to the external recording medium.

Various types of information such as various types of programs, data, tables, and databases may be stored in a recording medium and subjected to information processing. The CPU 1212 may execute various types of processing on the data read from the RAM 1214, including various types of operations, information processing, condition determination, conditional branching, unconditional branching, information retrieval/replacement, and the like, which are described throughout the disclosure and specified by a command sequence of a program, and writes back the results to the RAM 1214. The CPU 1212 may search for information in a file, a database, or the like in the recording medium. For example, in a case in which a plurality of entries each having an attribute value of a first attribute associated with an attribute value of a second attribute is stored in the recording medium, the CPU 1212 may search for an entry in which the attribute value of the first attribute matches a specified condition from the plurality of entries, read the attribute value of the second attribute stored in the entry, and thus acquire the attribute value of the second attribute associated with the first attribute satisfying a predetermined condition.

The programs or software modules described above may be stored in the computer 1200 or a computer readable storage medium near the computer 1200. A recording medium such as a hard disk or a RAM provided in a server system connected to a dedicated communication network or the Internet may be used as a computer readable storage medium, and provides a program to the computer 1200 via the network.

Blocks in the flowcharts and the block diagrams in the present embodiments may represent stages of a process in which an operation is performed or “units” of a device having a role of performing an operation. Specific stages and “units” may be implemented by a dedicated circuit, a programmable circuit supplied along with computer readable instructions stored on a computer readable storage medium, and/or a processor supplied along with computer readable instructions stored on a computer readable storage medium. Dedicated circuits may include digital and/or analog hardware circuits, and may include integrated circuits (ICs) and/or discrete circuits. Programmable circuits may include reconfigurable hardware circuits including, for example, AND, OR, XOR, NAND, NOR, and other logical operations, flip-flops, registers, and memory elements, such as field programmable gate arrays (FPGA) and programmable logic arrays (PLA).

Computer readable storage media may include any tangible device capable of storing instructions executed by a suitable device, and, as a result, a computer readable storage medium having instructions stored thereon has a product including instructions that may be executed to create means for executing operations designated in the flowcharts or block diagrams. Examples of the computer readable storage medium may include an electronic storage medium, a magnetic storage medium, an optical storage medium, an electromagnetic storage medium, and a semiconductor storage medium. More specific examples of the computer readable storage medium may include a Floppy (registered trademark) disk, a diskette, a hard disk, a random access memory (RAM), a read-only memory (ROM), an erasable programmable read-only memory (an EPROM or a flash memory), an electrically erasable programmable read-only memory (EEPROM), a static random access memory (SRAM), a compact disc read-only memory (CD-ROM), a digital versatile disk (DVD), a Blu-Ray (registered trademark) disk, a memory stick, and an integrated circuit card.

The computer readable instructions may include either source codes or object codes written in any combination of one or more programming languages, including assembler instructions, instruction-set-architecture (ISA) instructions, machine instructions, machine-dependent instructions, microcode, firmware instructions, state-setting data, or an object oriented programming language such as Smalltalk (registered trademark), or JAVA (registered trademark), C++, and conventional procedural programming languages such as the “C” programming language or similar programming languages.

The computer readable instructions may be provided to a processor of a general purpose computer, a special purpose computer, or another programmable data processing device, or a programmable circuit locally or via a local area network (LAN) or a wide area network (WAN) such as the Internet, in order to cause the processor of the general purpose computer, the special purpose computer, or another programmable data processing device or the programmable circuit to execute the computer readable instructions to generate means for executing operations designated in the flowcharts or the block diagrams. Examples of the processor include a computer processor, a processing unit, a microprocessor, a digital signal processor, a controller, and a microcontroller.

Although the technology of the disclosure has been described by using each embodiment, the technology of the disclosure is not limited to the scope disclosed in each embodiment. It is apparent to those skilled in the art that various modifications or improvements can be made to the above embodiments. It is apparent from the description of the claims that modes to which such modifications or improvements are added can also be included in the technical scope of the disclosure.

The order of execution of each piece of processing of operations, procedures, steps, stages, and the like in the devices, the systems, the programs, and the methods illustrated in the claims, the specification, and the drawings can be realized in any order unless “before”, “prior to”, or the like is explicitly stated, and unless the output of the previous processing is used in the later processing. Even in a case in which the operation flow in the claims, the specification, and the drawings is described by using “first,”, “next,”, and the like for convenience, this does not mean that it is essential to perform in this order.

### Other Embodiment 1

The action determination unit 236 generates action content of the robot for the action of the user and the emotion of the user or the emotion of the robot on the basis of an interaction function of allowing the user and the robot to interact with each other, and determines an action of the robot corresponding to the action content. In this case, the action determination unit 236 reflects a detection result obtained by detecting a change in the body temperature of the user in generation of an answer of the interaction function, estimation of the emotion of the user, and estimation of the emotion of the robot.

Specifically, the robot includes, for example, a thermo sensor as heat detection means for detecting the body temperature of the user, and detects a change in the body temperature of the user observed by the thermo sensor. The action determination unit 236 reflects the detection result in generation of an answer of the sentence generation model and estimation of the user emotion and the robot emotion by the emotion engine. For example, the action determination unit 236 determines that the user is “happy” in a case in which the entire body of the user is getting hot, and determines an action of the robot such that the robot performs a positive gesture or a positive speech corresponding to the determination. As a result, the action determination unit 236 can determine the action of the robot also in consideration of a change in human correspondence based on the emotion.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which
the action determination unit reflects a detection result obtained by detecting a change in the body temperature of the user in generation of an answer of the interaction function, estimation of the emotion of the user, and estimation of the emotion of the robot.

### Other Embodiment 2

The robot 100 of the present embodiment includes heat detection means for detecting the body temperature of the user 10.

The robot 100 of the present embodiment is configured to detect a change in the body temperature of the user 10 observed by the heat detection means.

The action determination unit 236 generates action content of the robot 100 with respect to the action of the user 10 and the emotion of the user 10 or the emotion of the robot 100 on the basis of the interaction function of allowing the user 10 and the robot 100 to interact with each other, and determines an action of the robot 100 corresponding to the action content. In this case, the action determination unit 236 reflects a detection result obtained by detecting the change in the body temperature of the user 10 in generation of an answer of the interaction function, estimation of the emotion of the user 10, and estimation of the emotion of the robot 100.

Specifically, such a robot 100 may include a thermo sensor as heat detection means, and may detect a change in the body temperature of the user 10 observed by the thermo sensor. The action determination unit 236 may reflect the detection result in generation of an answer of the sentence generation model and estimation of the emotion of the user 10 and an emotion of the robot 100 by the emotion engine. For example, the action determination unit 236 may determine that the user 10 is “angry” in a case in which the upper body of the user 10 becomes hot, and determine an action of the robot 100 such that the robot performs a soothing gesture or speech corresponding thereto. As a result, the action determination unit 236 can determine an action of the robot 100 also in consideration of a change in human correspondence based on the emotion.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which
the action determination unit reflects a detection result obtained by detecting a change in a body temperature of the user in generation of an answer of the interaction function, estimation of the emotion of the user, and estimation of the emotion of the robot.

### Other Embodiment 3

The robot 100 of the present embodiment includes heat detection means for detecting the body temperature of the user 10. The robot 100 of the present embodiment is configured to detect a change in the body temperature of the user 10 observed by the heat detection means.

The robot 100 of the present embodiment includes temperature control means for controlling the surface temperature of at least a part of the robot 100.

The action determination unit 236 generates action content of the robot 100 with respect to the action of the user 10 and the emotion of the user 10 or the emotion of the robot 100 on the basis of the interaction function of allowing the user 10 and the robot 100 to interact with each other, and determines an action of the robot 100 corresponding to the action content. In this case, the action determination unit 236 determines an action by reflecting a detection result obtained by detecting the change in the body temperature of the user 10 in generation of an answer of the interaction function, estimation of the emotion of the user 10, and estimation of the emotion of the robot 100, and the action determined by the action determination unit 236 includes an action of changing the surface temperature of at least a part of the robot 100.

Specifically, such a robot 100 may include a thermo sensor as the heat detection means, and may detect a change in the body temperature of the user 10 observed by the thermo sensor. The robot 100 may include a heater as the temperature control means. In the present embodiment, a case in which heating means such as a heater is used as the temperature control means will be described as an example, but the temperature control means is not limited thereto, and cooling means such as a cooler may be used. Such temperature control means may be provided for each part of the robot 100.

The action determination unit 236 may determine an action of the robot 100 by reflecting a detection result obtained by detecting the change in the body temperature of the user 10 in generation of an answer of the sentence generation model and estimation of the emotion of the user 10 and an emotion of the robot 100 by the emotion engine. In this case, the action determination unit 236 may select an action of changing the surface temperature of at least a part of the robot 100. The part of which the surface temperature is changed may be a part that may be touched by the user 10, and may be, for example, a hand or a face. For example, in a case in which the robot 100 has an emotion of “being happy”, the action determination unit 236 may select an action of warming the hand. In a case in which the robot 100 has an emotion of “being angry”, the action determination unit 236 may select an action of heating the face. This makes it possible to feel warm from the robot 100 in the case of giving a high five to the user 10 or in the case of comforting the user 10, so that it is possible to appeal to a human sense of expression more in line with the emotion of the user 10.

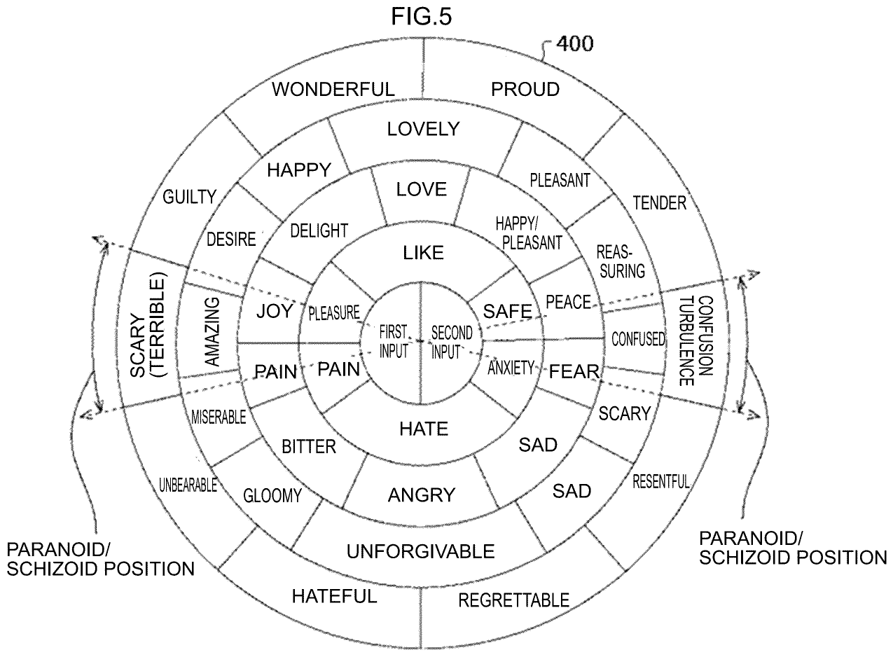

The emotion determination unit 232 may determine an emotion of the user according to a specific mapping. Specifically, the emotion determination unit 232 may determine an emotion of the user on the basis of an emotion map (see FIG. 5) that is a specific mapping.

FIG. 5 is a diagram illustrating an emotion map 400 on which a plurality of emotions are mapped. In the emotion map 400, emotions are disposed concentrically radially from the center. The closer to the center of the concentric circle, the more the emotion of the primitive state is disposed. Emotions indicating states and actions generated from the state of mind are disposed outside the concentric circle. The emotion is a concept including an affection and a mental state. On the left side of the concentric circle, emotions generated from reactions generally occurring in the brain are disposed. On the right side of the concentric circle, emotions induced by situation determination are generally disposed. In the up and down directions of the concentric circles, emotions generated from reactions generally occurring in the brain and induced by situational determination are disposed. The emotion of “pleased” is disposed on the upper side of the concentric circle, and the emotion of “displeased” is disposed on the lower side. As described above, in the emotion map 400, a plurality of emotions are mapped on the basis of a structure in which emotions occur, and emotions that are likely to occur at the same time are mapped close to each other.
(1) For example, in a case in which the emotion engine, which is the emotion determination unit 232 of the robot 100, detects an emotion at about 100 msec, determination of the reaction operation (for example, a quick-response) of the robot 100 may be performed at a timing at which a detection frequency is set to be similar to at least the detection frequency (100 msec) of the emotion engine, or may be performed at a timing at which the detection frequency is set to be higher than the detection frequency. The detection frequency of the emotion engine may be interpreted as a sampling rate.

The emotion is detected in about 100 msec, and the reaction operation (for example, a quick-response) is performed immediately in conjunction with the detection, whereby an unnatural quick-response is eliminated, and a dialogue in which natural air is read can be realized. The robot 100 performs a reaction operation (quick-response or the like) according to the directionality and the degree (intensity) of the mandala of the emotion map 400. The detection frequency (sampling rate) of the emotion engine is not limited to 100 ms, and may be changed according to a situation (for example, in the case of playing sports), the age of the user, or the like.
(2) In comparison with the emotion map 400, the directionality of the emotion and the intensity of the degree thereof may be set in advance, and the quick-response motion and the strength of the quick-response may be set. For example, in a case in which the robot 100 feels a sense of stability, safety, or the like, the robot 100 continues listening to the speech while nodding. In a case in which the robot 100 feels anxious, lost, or suspicious, the robot 100 may tilt its head or stop swinging its head.

These emotions are distributed in the 3:00 direction of the emotion map 400, and usually come and go between safety and anxiety. In the right half of the emotion map 400, situation recognition is superior to internal sensation, and thus gives a calm impression.
(3) In a case in which the robot 100 feels good in the case of receiving the compliment, a filler “Oh” may come in front of the line. In a case in which the robot feels heavy in the case of receiving harsh words, a filler “Ohh!” may come in front of the line. A physical reaction such as a gesture of the robot 100 crouching while saying “Ohh!” may be included. These emotions are distributed around 9:00 on the emotion map 400.
(4) In the left half of the emotion map 400, internal sensation (reaction) is superior to situation recognition. Therefore, an impression of unintentional reaction may be given.

In a case in which the robot 100 has a favorable feeling in situation recognition while remembering internal sensation (reaction) of satisfaction, the robot 100 may nod deeply while looking at the other party, or may say “yeah, yeah”. As described above, the robot 100 may generate a balanced favorable feeling to the other party, that is, an action such as allowance or tolerance to the other party. Such emotions are distributed around 12:00 in the emotion map 400.

In contrast, in a case in which the robot 100 feels antipathy in the situation recognition while remembering internal sensation (reaction) of displeasure, the robot 100 may shake its head sideways, and may turn red the LED of the eye and look at the other party in the case of feeling hatred. Such emotions are distributed around 6:00 in the emotion map 400.
(5) Since the inside of the emotion map 400 represents the inside of the mind and the outside of the emotion map 400 represents an action, the emotion is more visible (appears in the action) toward the outside of the emotion map 400.
(6) In a case in which the robot 100 listens to a person's speech while remembering the safety distributed around 3:00 on the emotion map 400, the robot 100 slightly shakes its head vertically and says “uh-huh”. However, in the direction of love around 12:00, the robot may perform strong nodding such as shaking its head deeply vertically.

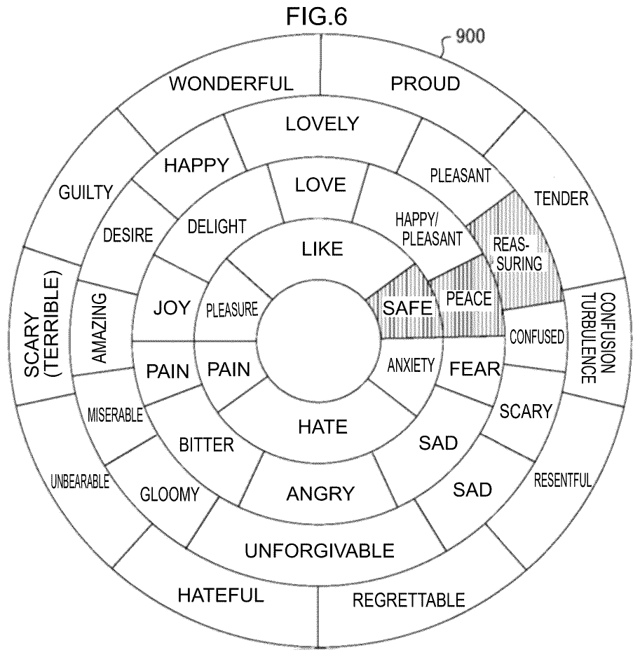

The emotion determination unit 232 inputs the information analyzed by the sensor module unit 210 and the recognized state of the user 10 to a neural network trained in advance, acquires an emotion value indicating each emotion indicated in the emotion map 400, and determines an emotion of the user 10. This neural network is trained in advance on the basis of a plurality of pieces of learning data that is a combination of the information analyzed by the sensor module unit 210 and the recognized state of the user 10 and the emotion value indicating each emotion indicated in the emotion map 400. This neural network is trained such that, as in an emotion map 900 illustrated in FIG. 6, emotions disposed close to each other have close values. FIG. 6 illustrates an example in which a plurality of emotions such as “safe”, “calm”, and “reassuring” have close emotion values.

The emotion determination unit 232 may determine an emotion of the robot 100 according to a specific mapping. Specifically, the emotion determination unit 232 inputs the information analyzed by the sensor module unit 210, the state of the user 10 recognized by the user state recognition unit 230, and the state of the robot 100 to a neural network trained in advance, acquires an emotion value indicating each emotion indicated in the emotion map 400, and determines an emotion of the robot 100. This neural network is trained in advance on the basis of a plurality of pieces of learning data that is a combination of the information analyzed by the sensor module unit 210, the recognized state of the user 10, the state of the robot 100, and the emotion value indicating each emotion illustrated in the emotion map 400. For example, the neural network is trained on the basis of learning data indicating that the emotion value “3” of “happy” is obtained in a case in which the robot 100 is recognized as being cared by the user 10 from the output of the touch sensor (not illustrated), and learning data indicating that the emotion value “3” of “angry” is obtained in a case in which the robot 100 is recognized as being hit by the user 10 from the output of the acceleration sensor (not illustrated). This neural network is trained such that, as in the emotion map 900 illustrated in FIG. 6, emotions disposed close to each other have close values.

The action determination unit 236 adds a fixed sentence for inquiry about the action content of the robot corresponding to the action of the user to text representing the action of the user, the emotion of the user, and the emotion of the robot, and inputs the text to the sentence generation model having the interaction function, thereby generating the action content of the robot.

For example, the action determination unit 236 acquires text indicating the state of the robot 100 from the emotion of the robot 100 determined by the emotion determination unit 232 by using an emotion table as illustrated in Table 1. Here, in the emotion table, an index number is assigned to each emotion value for each type of emotion, and a text indicating the state of the robot 100 is stored for each index number.

In a case in which the emotion of the robot 100 determined by the emotion determination unit 232 corresponds to the index number “2”, text “very pleasant state” is obtained. In a case in which the emotion of the robot 100 corresponds to a plurality of index numbers, a plurality of pieces of text indicating the state of the robot 100 are obtained. An emotion table as illustrated in Table 2 is prepared for an emotion of the user 10.

Here, in a case in which the action of the user is to say “I succeeded thanks to you, thanks.”, the emotion of the robot 100 is the index number “2”, and the emotion of the user 10 is the index number “3”, a sentence such as “The robot is in a very pleasant state. The user is in a normally pleasant state. The user said, “I succeeded thanks to you, thanks.”. How do you answer as a robot?”
is input to the sentence generation model to acquire the action content of the robot. The action determination unit 236 determines an action of the robot from the action content.

TABLE 1

| Index | | Emotion | |
|---|---|---|---|
| Number | Type of Emotion | Value | State of Robot |
| 1 | Pleasant | 5 | Very Pleasant State |
| 2 | Pleasant | 4 | Extremely Pleasant State |
| 3 | Pleasant | 3 | Normally Pleasant State |
| 4 | Pleasant | 2 | Slightly Pleasant State |
| 5 | Pleasant | 1 | Only a Little Pleasant State |
| . . . | . . . | . . . | . . . |

TABLE 2

| Index | | Emotion | |
|---|---|---|---|
| number | Type of Emotion | Value | State of Robot |
| 1 | Pleasant | 5 | Very Pleasant State |
| 2 | Pleasant | 4 | Extremely Pleasant State |
| 3 | Pleasant | 3 | Normally Pleasant State |
| 4 | Pleasant | 2 | Slightly Pleasant State |
| 5 | Pleasant | 1 | Only a Little Pleasant |
| . . . | . . . | . . . | . . . |

As described above, the action determination unit 236 determines the action content of the robot 100 in accordance with the state related to the emotion of the robot 100 determined in advance for each type of the emotion of the robot 100 and for each intensity of the emotion, and the action of the user 10. In this embodiment, the speech content of the robot 100 in a case in which an interaction with the user 10 is performed can be branched according to the state related to the emotion of the robot 100. That is, since the robot 100 can change the action of the robot according to the index number corresponding to the emotion of the robot, the user has an impression that the robot has a mind, and is promoted to take an action such as talking to the robot.

The action determination unit 236 may generate the action content of the robot by adding a fixed sentence for inquiry about the action content of the robot corresponding to the action of the user and inputting the fixed sentence to the sentence generation model having the interaction function after adding not only the text indicating the action of the user, the emotion of the user, and the emotion of the robot but also the text indicating the content of the history data 222. As a result, the robot 100 can change the action of the robot according to the history data indicating the emotion or the action of the user, and thus, the user has an impression that the robot has personality, and is promoted to take an action such as talking to the robot. The history data may further include an emotion or an action of the robot.

The emotion determination unit 232 may determine an emotion of the robot 100 on the basis of the action content of the robot 100 generated by the sentence generation model. Specifically, the emotion determination unit 232 inputs the action content of the robot 100 generated by the sentence generation model to the neural network trained in advance, acquires the emotion value indicating each emotion indicated in the emotion map 400, integrates the acquired emotion value indicating each emotion and the emotion value indicating each emotion of the current robot 100, and updates the emotion of the robot 100. For example, the acquired emotion value indicating each emotion and the emotion value indicating each emotion of the current robot 100 are averaged and integrated. This neural network is trained in advance on the basis of a plurality of pieces of learning data that is a combination of text representing the action content of the robot 100 generated by the sentence generation model and the emotion value representing each emotion illustrated in the emotion map 400.

For example, in a case in which, as the action content of the robot 100 generated by the sentence generation model, the speech content of the robot 100, “That was good. Lucky you.” is obtained, when the text representing the speech content is input to the neural network, a great value is obtained as the emotion value of the emotion “happy”, and the emotion of the robot 100 is updated so that the emotion value of the emotion “happy” increases.

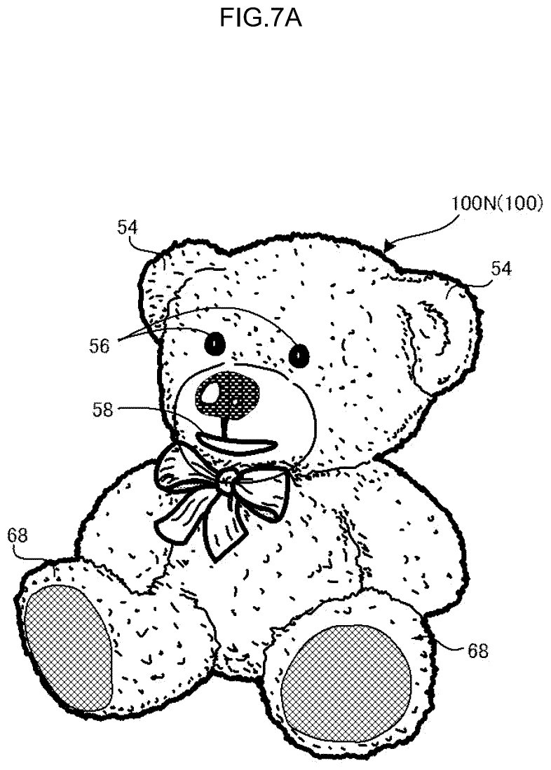

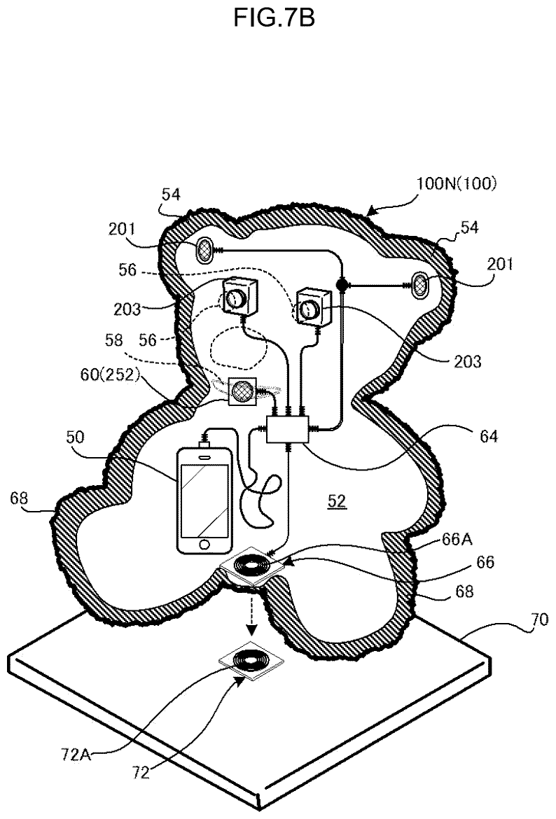

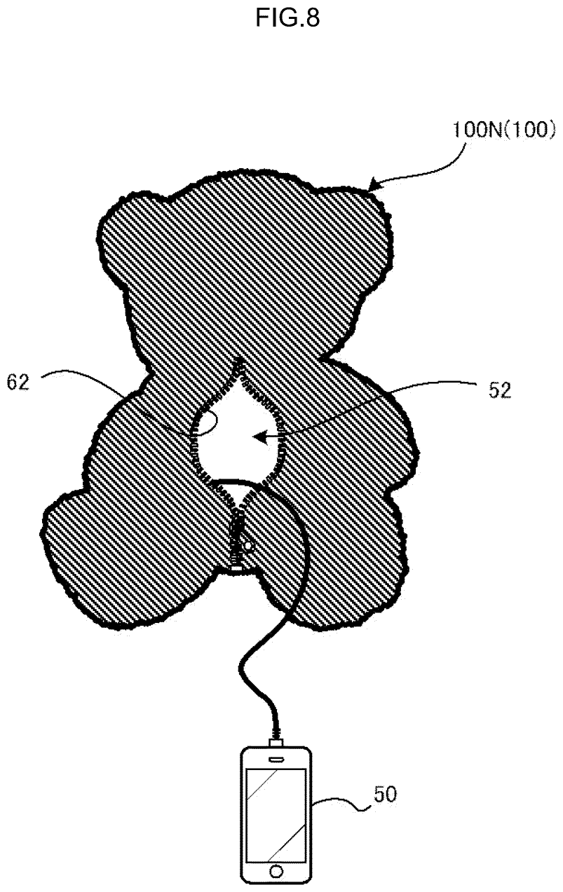

The robot 100 may be mounted on a stuffed toy, or may be applied to a control device connected wirelessly or by wire to a control target device (speaker or camera) mounted on the stuffed toy. In this case, specifically, the robot is configured as follows. For example, the robot 100 may be applied to a cohabiter (specifically, a stuffed toy 100N illustrated in FIGS. 7 and 8) who advances a dialogue with the user 10 on the basis of information regarding daily life while spending daily life with the user 10 or provides information matching the hobby of the user 10. In the present embodiment (another embodiment), an example in which the control portion of the robot 100 is applied to a smartphone 50 will be described.

The smartphone 50 functioning as a control portion of the robot 100 is detachably attached to the stuffed toy 100N having a function as an input/output device of the robot 100, and the input/output device and the accommodated smartphone 50 are connected inside the stuffed toy 100N.

As illustrated in FIG. 7(A), the stuffed toy 100N has the shape of a bear covered with a soft cloth fabric in the present embodiment (an embodiment mounted on the stuffed toy). As illustrated in FIG. 7(B), in a space portion 52 formed inside the stuffed toy, the microphone 201 (see FIG. 2) of the sensor unit 200 is disposed in a portion corresponding to an ear 54 as an input/output device, the 2D camera 203 of the sensor unit 200 is disposed in a portion corresponding to the eye 56 (see FIG. 2), and a speaker 60 configuring a part of the control target 252 (see FIG. 2) is disposed in a portion corresponding to a mouth 58. The microphone 201 and the speaker 60 are not necessarily separated from each other, and may be an integrated unit. In the case of the unit, it is preferable to dispose the unit at a position where the speech can be heard naturally, such as the position of the nose of the stuffed toy 100N. Although the case in which the stuffed toy 100N has an animal shape has been described as an example, the disclosed technology is not limited thereto. The stuffed toy 100N may have a shape of a specific character.

The smartphone 50 has a function as the sensor module unit 210, a function as the storage unit 220, a function as the user state recognition unit 230, a function as the emotion determination unit 232, a function as the action recognition unit 234, a function as the action determination unit 236, a function as the storage control unit 238, a function as the action control unit 250, and a function as the communication processing unit 280 illustrated in FIG. 2.

As illustrated in FIG. 8, a fastener 62 is attached to a part (for example, the back portion) of the stuffed toy 100N, and the outside and the space portion 52 communicate with each other by opening the fastener 62.

Here, the smartphone 50 is accommodated in the space portion 52 from the outside and is connected to each input/output device through USB connection via a USB hub 64 (see FIG. 7(B)), so that a function equivalent to that of the robot 100 illustrated in FIG. 1 can be provided.

A non-contact power receiving plate 66 is connected to the USB hub 64. A power receiving coil 66A is incorporated in the power receiving plate 66. The power receiving plate 66 is an example of a wireless power receiver that receives wireless power supply.

The power receiving plate 66 is disposed near root portions 68 of both feet of the stuffed toy 100N, and is located closest to a placement base 70 in a case in which the stuffed toy 100N is placed on the placement base 70. The placement base 70 is an example of an external wireless power transmitter.

The stuffed toy 100N placed on the placement base 70 can be appreciated as an ornament in a natural state.

This root portion is formed to be thinner than the surface layer thickness of the stuffed toy 100N in other parts, and is held in a state closer to the placement base 70.

The placement base 70 includes a charging pad 72. A power transmitting coil 72A is incorporated in the charging pad 72. In a case in which the power transmitting coil 72A transmits a signal to search a power receiving coil 66A of the power receiving plate 66, and the power receiving coil 66A is found, a current flows through the power transmitting coil 72A to generate a magnetic field, and the power receiving coil 66A reacts to the magnetic field to start electromagnetic induction. As a result, a current flows through the power receiving coil 66A, and power is stored in a battery (not illustrated) of the smartphone 50 via the USB hub 64.

That is, since the smartphone 50 is automatically charged by placing the stuffed toy 100N as an ornament on the placement base 70, it is not necessary to take out the smartphone 50 from the space portion 52 of the stuffed toy 100N for charging.

In the present embodiment (the embodiment mounted on the stuffed toy), the smartphone 50 is accommodated in the space portion 52 of the stuffed toy 100N and connected by wire (USB connection), but the disclosed technology is not limited thereto. For example, a control device having a wireless function (for example, “Bluetooth (registered trademark)”) may be accommodated in the space portion 52 of the stuffed toy 100N, and the control device may be connected to the USB hub 64. In this case, the smartphone 50 and the control device wirelessly communicate with each other without inserting the smartphone 50 into the space portion 52, and the external smartphone 50 is connected to each input/output device via the control device, so that a function equivalent to that of the robot 100 illustrated in FIG. 1 can be provided. The control device in which the control device is accommodated in the space portion 52 of the stuffed toy 100N and the external smartphone 50 may be connected by wire.

In the present embodiment (the embodiment mounted on the stuffed toy), the stuffed toy 100N having a bear shape has been exemplified, but may be another animal or a doll, or may have a shape of a specific character. The clothes may be changeable. A material of the skin is not limited to the cloth fabric, and may be other materials such as soft vinyl, but is preferably a soft material.

A monitor may be attached to the skin of the stuffed toy 100N, and the control target 252 that provides information to the user 10 through vision may be added. For example, the eyes 56 may be used as a monitor to express happiness, anger, sadness, and pleasure with images captured in the eyes, or a window through which the monitor of the built-in smartphone 50 transmits may be provided in the abdomen. The eyes 56 may be used as a projector to express happiness, anger, sadness, and pleasure by using an image projected on a wall surface.

According to another embodiment, the existing smartphone 50 is placed in the stuffed toy 100N, and the camera 203, the microphone 201, the speaker 60, and the like are extended from the place to appropriate positions through the USB connection.

For wireless charging, the smartphone 50 and the power receiving plate 66 are connected through USB connection, and the power receiving plate 66 is disposed on the outside as much as possible when viewed from the inside of the stuffed toy 100N.

In order to use the wireless charging of the smartphone 50, it is necessary to dispose the smartphone 50 on the outside as much as possible when viewed from the inside of the stuffed toy 100N, and the stuffed toy 100N is rough in the case of being touched from the outside.

Therefore, the smartphone 50 is disposed at the center of the stuffed toy 100N as much as possible, and the wireless charging function (power receiving plate 66) is disposed outside as viewed from the inside of the stuffed toy 100N as much as possible. The camera 203, the microphone 201, the speaker 60, and the smartphone 50 receive wireless power supply via the power receiving plate 66.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit determines the action by reflecting a detection result obtained by detecting a change in a body temperature of the user in generation of an answer of the interaction function, estimation of the emotion of the user, and estimation of the emotion of the robot, and
the action determined by the action determination unit includes an action of changing a surface temperature of at least a part of the robot.

### (Supplementary Note 2)

The action control system according to Supplementary Note 1, in which the robot is mounted on a stuffed toy or is connected to a control target device mounted on the stuffed toy in a wireless or wired manner.

### Other Embodiment 4

The robot 100 of the present embodiment includes extraction means for extracting the preference of the user 10 from the conversation of the user 10.

The action determination unit 236 generates action content of the robot 100 with respect to the action of the user 10 and the emotion of the user 10 or the emotion of the robot 100 on the basis of the sentence generation model having the interaction function of allowing the user 10 and the robot 100 to interact with each other, and determines an action of the robot 100 corresponding to the action content. In this case, the action determination unit 236 reflects the preference of the user 10 extracted from the conversation of the user 10 in generation of an answer of the interaction function, estimation of the emotion of the user 10, and estimation of the emotion of the robot 100.

Specifically, such a robot 100 may include a preference learning engine as the extraction means, and may extract the preference of the user 10 by inputting the conversation of the user 10 to the preference learning engine. The “conversation of the user 10” here may be interpreted to include a conversation between the user 10 and another robot, a conversation between the users 10, and a soliloquy of the user 10, in addition to the interaction between the user 10 and the robot 100 via the sentence generation model. That is, the robot 100 may extract the preference of the user 10 from the conversation of the user 10 that the robot 100 itself has attended without being a party, in addition to the interaction with the user 10 in which the robot 100 itself is a party.

The action determination unit 236 may reflect the preference of the user 10 extracted from the conversation of the user 10 in generation of an answer of the sentence generation model and estimation of the emotion of the user 10 and an emotion of the robot 100 by the emotion engine. For example, the robot 100 may ascertain a baseball team that the user 10 supports from the conversation of the user 10. In a case in which the supporting baseball team has won, the action determination unit 236 may determine an action of the robot 100 to express a feeling of joy together with the answer of “Yay!”. On the other hand, in a case in which the competitor team has won, the action determination unit 236 may determine an action of the robot 100 to express the emotion of anger together with the answer “Frustrated!”. As a result, the robot 100 can respond to the user 10 whom the robot meets for the first time by a different reaction for each user 10, so that the user experience can be improved.

In the present embodiment, for example, in a case in which the action of the user is to speak “The OO team has won!”, the emotion of the robot 100 is the index number “2”, and the emotion of the user 10 is the index number “3”, a sentence such as “The robot is in a very pleasant state. The user is in a normally pleasant state. The user says “The OO team has won!”. How do you answer as a robot?” is input to the sentence generation model to acquire the action content of the robot. The action determination unit 236 determines an action of the robot from the action content.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of a sentence generation model having an interaction function of allowing the user and the robot to interact with each other, and determines an action of the robot corresponding to the action content, in which
the action determination unit reflects a preference of the user extracted from a conversation of the user in generation of an answer of the interaction function, estimation of the emotion of the user, and estimation of the emotion of the robot.

### (Supplementary Note 2)

The action control system according to Supplementary Note 1, in which the robot is mounted on a stuffed toy or is connected to a control target device mounted on the stuffed toy in a wireless or wired manner.

### Other Embodiment 5

The robot 100 of the present embodiment includes estimation means for estimating a culture area (also referred to as a language area) of the user 10.

The action determination unit 236 generates action content of the robot 100 with respect to the action of the user 10 and the emotion of the user 10 or the emotion of the robot 100 on the basis of the interaction function of allowing the user 10 and the robot 100 to interact with each other, and determines an action of the robot 100 corresponding to the action content. In this case, the action determination unit 236 reflects the estimated culture area of the user 10 in generation of an answer of the interaction function, estimation of the emotion of the user 10, and estimation of the emotion of the robot 100.

Specifically, such a robot 100 may estimate the culture area of the user 10 by using various methods. For example, the estimation means may estimate the culture area of the user 10 from a conversation of the user 10. The “conversation of the user 10” here may be interpreted to include a conversation between the user 10 and another robot, a conversation between the users 10, and a soliloquy of the user 10, in addition to the interaction between the user 10 and the robot 100 via the sentence generation model. That is, the robot 100 may estimate the culture area of the user 10 from the conversation of the user 10 that the robot 100 itself has attended without being a party, in addition to the interaction with the user 10 in which the robot 100 itself is a party. As an example, the estimation means may estimate that the culture area of the user 10 is the Kansai area in a case in which the user 10 frequently talks about Osaka prefecture in a conversation or in a case in which local information of Osaka prefecture is said as a topic. The estimation means may estimate that the culture area of the user 10 is the Kansai area in a case in which the user uses the Kansai dialect in the conversation. Alternatively or in addition thereto, the estimation means may estimate the culture area of the user 10 on the basis of position information. As an example, the estimation means may store in advance a culture area map in which the position information and the culture area are associated with each other, and in a case in which a position measured by positioning means is associated with the Kansai area, the estimation means may estimate that the culture area of the user 10 is the Kansai area.

The action determination unit 236 may reflect the estimated culture area of the user 10 in generation of an answer of the sentence generation model and estimation of the emotion of the user 10 and an emotion of the robot 100 by the emotion engine. For example, in a case in which it is estimated that the culture area of the user 10 is the Kansai area, the action determination unit 236 may determine an action of the robot 100 so that the robot makes a gesture of putting a thrust or makes a speech such as “Why?”. As a result, according to the robot 100 of the present embodiment, since it is possible to act in accordance with the residential culture of the user 10, it is possible to improve the user experience.

In the present embodiment, for example, in a case in which the action of the user is to speak “It is hot in the summer, the chest is excited.”, the emotion of the robot 100 is the index number “2”, and the emotion of the user 10 is the index number “3”, a sentence such as “The robot is in a very pleasant state. The user is in a normally pleasant state. The user says “It is hot in the summer, the chest is excited.”. How do you answer as a robot?” is input to the sentence generation model to acquire the action content of the robot. The action determination unit 236 determines an action of the robot from the action content.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which
the action determination unit reflects an estimated culture area of the user in generation of an answer of the interaction function, estimation of the emotion of the user, and estimation of the emotion of the robot.

### (Supplementary Note 2)

The action control system according to Supplementary Note 1, in which the robot is mounted on a stuffed toy or is connected to a control target device mounted on the stuffed toy in a wireless or wired manner.

### Other Embodiment 6

The robot 100 of the present embodiment has a function of recognizing characteristic information of the user 10. Specifically, the robot 100 can track the user's personality, preference, habit, motion, idea, action, conversation content, emotion, and the like, which are the characteristic information, as a history.

In order to acquire the above characteristic information, the robot 100 may have various functions. Specifically, the robot 100 may have a camera function capable of capturing the face of the user 10, a microphone function capable of acquiring the voice of the user 10, a heat detection function capable of detecting the body temperature of the user 10, for example, thermography, and the like, as functions for acquiring the user's personality, motion, action, conversation content, emotions, and the like.

The robot 100 may have a communication function capable of acquiring various types of information from the SNS of the user as a function for acquiring the user's preference, habit, idea, action, and the like. The characteristic information collected through the above various functions may be stored in association with a specific user in the robot 100, a storage of the server 300, or the like.

The robot 100 has a function of acquiring environment information at the time of acquiring the various types of characteristic information. The environment information here indicates a situation such as a temperature, a brightness, weather, a status, time, a season, a place, and the like at the time of acquiring the various types of characteristic information. In order to acquire the environment information, the robot 100 may include a temperature sensor, an illuminance sensor, a timer, and position detection means such as a GPS.

The action determination unit 236 controls the robot 100 or the like to collect the characteristic information of the user and the environment information at the time of acquiring the characteristic information. In a case in which the user 10 starts the interaction with the robot 100, the action determination unit 236 predicts interaction content of the user 10 on the basis of the collected characteristic information and environment information, and environment information acquired by the robot 100 at that time, and determines a speech having content including a result of the prediction as an action of the robot 100. The action of the robot 10 by the action determination unit 236 is determined in consideration of the emotion of the user or the emotion of the robot determined by the emotion determination unit 232 as will be described later.

The robot 100 can execute a speech having content including the prediction result, determined by the action determination unit 236 for the user 10 before the user 10 speaks to the robot 100.

The speech content of the robot 100 may be determined by using the sentence generation model described above. The start of the interaction of the user 10 with the robot 100 can be specified, for example, by detecting that the user 10 approaches or by detecting that the user 10 visually recognizes the display device 1218 of the robot 100 by using the camera function of the robot 100.

In other words, in the system according to the present embodiment, first, all pieces of the characteristic information of the user 10 such as a personality, a preference, a habit, motion, an idea, an action, conversation content, and an emotion of the user 10 is stored and tracked as a history, and situations such as a temperature, a brightness, weather, a status, time, a season, and a place at the time of storing the characteristic information are collected. In a case in which the user 10 intends to have a conversation with the robot 100, before the user 10 speaks to the robot 100, the robot 100 side estimates the content of the conversation with the user 10 from the situations such as the temperature, the brightness, the weather, the status, the time, the season, and the place at that time, and the robot 100 says what the user 10 desires in advance.

By performing the above speech, the user 10 does not need to ask the robot 100 a first question, and can recognize as if the robot 100 understood the user 10 well.

Such a robot 100 is particularly effective in a use application in which a plurality of interactions can be performed between a specific user 10 and the robot 100, for example, in an office or a care site.

In the present embodiment, for example, in a case in which the action of the user is to say “Good morning”, the emotion of the robot 100 is the index number “2”, and the emotion of the user 10 is the index number “3”, a sentence such as “The robot is in a very pleasant state. The user is in a normally pleasant state. The user said “Good morning”. How do you answer as a robot?” is input to the sentence generation model to acquire the action content of the robot. The action determination unit 236 determines an action of the robot from the action content.

The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which
the action determination unit collects characteristic information of the user and environment information at the time of acquiring the characteristic information, predicts interaction content of the user when the user starts an interaction with the robot on the basis of the collected characteristic information and environment information, and environment information at the time at which the user starts the interaction with the robot, and determines a speech having content including a result of the prediction as the action of the robot.

### (Supplementary Note 2)

The action control system according to Supplementary Note 1, in which the robot executes the speech having the content including the result of the prediction determined by the action determination unit on the user before the user speaks to the robot.

### (Supplementary Note 3)

The action control system according to Supplementary Note 1 or 2, in which the robot is mounted on a stuffed toy, or is connected to a control target device mounted on the stuffed toy in a wireless or wired manner.

### Other Embodiment 7

The action determination unit 236 may analyze a social networking service (SNS) related to the user and recognize what the user is interested in on the basis of a result of the analysis. Examples of the SNS related to the user include an SNS that the user usually browses or an SNS of the user. In this case, the action determination unit 236 may propose a spot and/or an event recommended to the user at the current position of the user. In particular, in a case in which the user goes to a place that the user does not know at all, the user's convenience can be achieved by proposing a spot and/or an event recommended to the user. In this case, in a case in which the user selects a plurality of spots and/or a plurality of events in advance, the action determination unit 236 may determine the most efficient route for going around the plurality of spots and/or the plurality of events in consideration of the congestion status on the day and the like. In this case, the action determination unit 236 may cause the robot 100 to act together with the user to guide the user to a spot and/or an event. The guidance content may include not only the selected spot and/or event, but also guidance content similar to what a human tour guide usually provides, a history of a town, buildings visible from a road, and the like on the way. A language for the guidance is not limited to Japanese, and can be set to any language. The following supplementary notes regarding the above embodiment will be disclosed.

### (Supplementary Note 1)

An action control system including:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content, in which the action determination unit analyzes an SNS related to the user, and recognizes a thing in which the user is interested on the basis of a result of the analysis.

### (Supplementary Note 2)

The action control system according to Supplementary Note 1, in which the action determination unit proposes a spot and/or an event recommended to the user at a current position of the user on the basis of the item.

### (Supplementary Note 3)

The action control system according to Supplementary Note 1 or 2, in which the action determination unit derives, among a plurality of spots and/or a plurality of events selected in advance, a route for going around the plurality of spots and/or the plurality of events according to at least a current congestion status, and determines action content of the robot to circulate the route.

### (Supplementary Note 4)

The action control system according to Supplementary Note 3, in which the action determination unit provides guidance regarding at least one spot and/or event among the plurality of spots and/or the plurality of events in a predetermined language.

### Other Embodiment 8

The robot 100 (the present embodiment corresponds to the smartphone 50 accommodated in the stuffed toy 100N) according to the present embodiment executes the following processing.

The action determination unit 236 determines an action of the robot 100 corresponding to a user state and an emotion of the user 10 or an emotion of the robot 100 on the basis of a sentence generation model having an interaction function of allowing the user 10 and the robot 100 to interact with each other. In this case, the action determination unit 236 reflects a detection result obtained by detecting at least one of contact of the user 10 or a pressure change due to the contact in at least one of generation of an answer of the interaction function, estimation of the emotion of the user 10, or estimation of the emotion of the robot 100.

Specifically, the robot 100 may include, for example, a touch sensor as contact detection means for detecting contact of the user 10. As an example, such a touch sensor may be provided at a nose portion of the stuffed toy 100N. The robot 100 may include, for example, an air pressure sensor as pressure detection means for detecting a pressure change due to the contact of the user 10. As an example, such an air pressure sensor may be provided at a hand portion of the stuffed toy 100N. The robot 100 may have pressure control means capable of controlling the internal air pressure. The action determination unit 236 reflects the detection result detected by the touch sensor or the air pressure sensor in at least one of generation of an answer of the interaction function of the sentence generation model or estimation of the emotion of the user 10 or estimation of the emotion of the robot 100 by the emotion engine.

For example, in a case in which the user 10 shakes hands, the action determination unit 236 can read the emotion of the user 10 from the degree of force of the hand of the user 10. In response to this, the action determination unit 236 can transmit the warmness to the user 10 by controlling the air pressure level in the robot 100. The action determination unit 236 can feel the love of the user 10 in a case in which the nose of the stuffed toy is kissed by the user. The hand portion of the stuffed toy 100N may be further provided with, for example, a temperature sensor as temperature detection means for detecting the body temperature of the user 10. In this case, the action determination unit 236 can also read the emotion of the user 10 in consideration of the body temperature of the user 10 in addition to the degree of force of the hand of the user 10.

In the present embodiment, for example, in a case in which the action of the user is to say “Bear, I love you!”, the emotion of the robot 100 is the index number “2”, and the emotion of the user 10 is the index number “3”, a sentence such as “The robot is in a very pleasant state. The user is in a normally pleasant state. The user says “Bear, I love you!”. How do you answer as a robot?” is input to the sentence generation model to acquire the action content of the robot. The action determination unit 236 determines an action of the robot from the action content.

The following supplementary notes regarding the above embodiment will be disclosed.

### (Supplementary Note 1)

An action control system including:

a user state recognition unit that recognizes a user state including an action of a user;
an emotion determination unit that determines an emotion of the user or an emotion of a robot; and
an action determination unit that determines an action of the robot corresponding to the user state and the emotion of the user or the emotion of the robot on the basis of a sentence generation model having an interaction function of allowing the user and the robot to interact with each other, in which
the action determination unit reflects a detection result obtained by detecting at least one of contact of the user or a pressure change due to the contact in at least one of generation of an answer of the interaction function, estimation of the emotion of the user, or estimation of the emotion of the robot.

### (Supplementary Note 2)

The action control system according to Supplementary Note 1, in which the robot is mounted on a stuffed toy or is connected to a control target device mounted on the stuffed toy in a wireless or wired manner.

### (Supplementary Note 3)

The action control system according to Supplementary Note 2, in which the control target device is a speaker, and a microphone or a camera is mounted on the stuffed toy.

### (Supplementary Note 4)

The action control system according to Supplementary Note 3, in which the camera is attached to an eye configuring a face of the stuffed toy, the microphone is attached to an ear, and the speaker is attached to a mouth.

### (Supplementary Note 5)

The action control system according to Supplementary Note 2, in which a wireless power receiver that receives wireless power supply from an external wireless power transmitter is disposed inside the stuffed toy, and the control target device or the robot receives power via the wireless power receiver.

### Second Embodiment

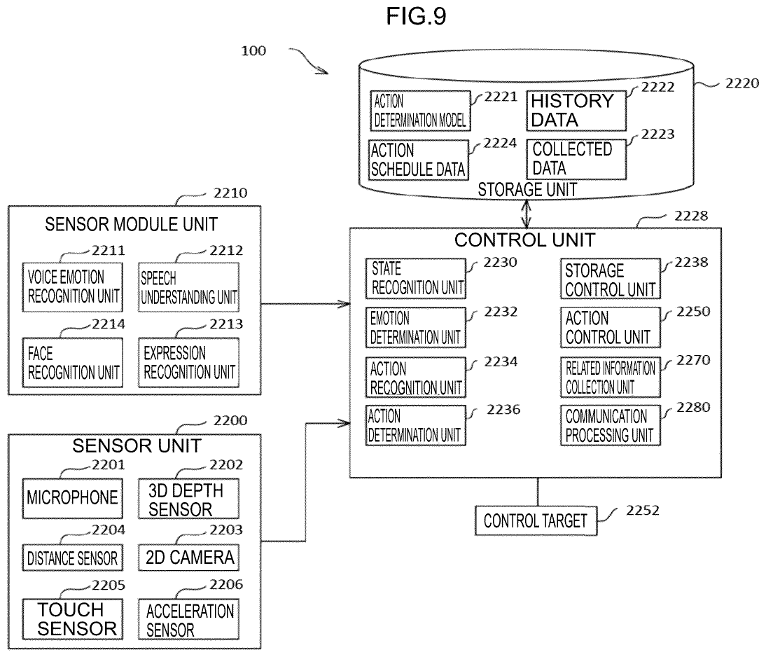

FIG. 9 schematically illustrates a functional configuration of a robot 100. The robot 100 includes a sensor unit 2200, a sensor module unit 2210, a storage unit 2220, a control unit 2228, and a control target 2252. The control unit 2228 includes a state recognition unit 2230, an emotion determination unit 2232, an action recognition unit 2234, an action determination unit 2236, a storage control unit 2238, an action control unit 2250, a related information collection unit 2270, and a communication processing unit 2280.

Examples of the control target 2252 include a display device, a speaker, an LED of an eye portion, and motors that drive an arm, a hand, a foot, and the like. The posture and the action of the robot 100 are controlled by controlling motors for arms, hands, and feet. Some of the emotions of the robot 100 can be expressed by controlling these motors. The expression of the robot 100 can be expressed by controlling a light emission state of the LED of the eye portion of the robot 100. The posture, the gesture, and the expression of the robot 100 are examples of the attitude of the robot 100.

The sensor unit 2200 includes a microphone 2201, a 3D depth sensor 2202, a 2D camera 2203, a distance sensor 2204, a touch sensor 2205, and an acceleration sensor 2206. The microphone 2201 continuously detects a voice and outputs voice data. The microphone 2201 may be provided on the head of the robot 100 and may have a function of performing binaural recording. The 3D depth sensor 2202 analyzes infrared patterns from infrared images continuously captured by an infrared camera by continuously applying the infrared patterns, and detects an outline of an object. The 2D camera 2203 is an example of an image sensor. The 2D camera 2203 captures an image with visible light and generates image information of visible light. The distance sensor 2204 detects a distance to an object by emitting, for example, laser light or ultrasonic waves. The sensor unit 2200 may further include a clock, a gyro sensor, a sensor for motor feedback, and the like.

Among the constituents of the robot 100 illustrated in FIG. 9, the constituents other than the control target 2252 and the sensor unit 2200 are examples of the constituents included in the action control system included in the robot 100. The action control system of the robot 100 controls the control target 2252.

The storage unit 2220 includes an action determination model 2221, history data 2222, collected data 2223, and action schedule data 2224. The history data 2222 includes past emotion values of the user 10, past emotion values of the robot 100, and a history of actions, and specifically includes a plurality of pieces of event data including the emotion values of the user 10, the emotion values of the robot 100, and the actions of the user 10. The data including the action of the user 10 includes a camera image representing the action of the user 10. The emotion values and the history of actions are recorded for each user 10 by being associated with identification information of the user 10, for example. At least a part of the storage unit 2220 is implemented by a storage medium such as a memory. A person DB that stores a face image of the user 10, attribute information of the user 10, and the like may be included. Among the constituents of the robot 100 illustrated in FIG. 9, the functions of the constituents other than the control target 2252, the sensor unit 2200, and the storage unit 2220 can be realized by a CPU operating on the basis of a program. For example, the functions of these constituents can be implemented as the operation of the CPU by basic software (OS) and a program operating on the OS.

The sensor module unit 2210 includes a voice emotion recognition unit 2211, a speech understanding unit 2212, an expression recognition unit 2213, and a face recognition unit 2214. Information detected by the sensor unit 2200 is input to the sensor module unit 2210. The sensor module unit 2210 analyzes information detected by the sensor unit 2200 and outputs an analysis result to the state recognition unit 2230.

The voice emotion recognition unit 2211 of the sensor module unit 2210 analyzes the voice of the user 10 detected by the microphone 2201 to recognize the emotion of the user 10. For example, the voice emotion recognition unit 2211 extracts a feature amount such as a frequency component of the voice and recognizes the emotion of the user 10 on the basis of the extracted feature amount. The speech understanding unit 2212 analyzes the voice of the user 10 detected by the microphone 2201 and outputs text information indicating the speech content of the user 10.

The expression recognition unit 2213 recognizes the expression of the user 10 and the emotion of the user 10 from the image of the user 10 captured by the 2D camera 2203. For example, the expression recognition unit 2213 recognizes the expression and emotion of the user 10 on the basis of the shapes, positional relationships, and the like of the eyes and the mouth.

The face recognition unit 2214 recognizes the face of the user 10. The face recognition unit 2214 recognizes the user 10 by matching a face image stored in a person DB (not illustrated) with a face image of the user 10 captured by the 2D camera 2203.

The state recognition unit 2230 recognizes a state of the user 10 on the basis of the information analyzed by the sensor module unit 2210. For example, processing mainly related to perception is performed by using the analysis result of the sensor module unit 2210. For example, perception information such as “There is one father.” and “The probability that the father does not smile is 90%.” is generated. Processing of understanding the meaning of the generated perception information is performed. For example, semantic information such as “One father looks lonely.” is generated.

The state recognition unit 2230 recognizes a state of the robot 100 on the basis of the information detected by the sensor unit 2200. For example, the state recognition unit 2230 recognizes a remaining battery level of the robot 100, a brightness of the surrounding environment of the robot 100, or the like as the state of the robot 100.

The emotion determination unit 2232 determines an emotion value indicating the emotion of the user 10 on the basis of the information analyzed by the sensor module unit 2210 and the state of the user 10 recognized by the state recognition unit 2230. For example, the information analyzed by the sensor module unit 2210 and the recognized state of the user 10 are input to a neural network trained in advance, and an emotion value indicating the emotion of the user 10 is acquired.

Here, the emotion value indicating the emotion of the user 10 is a value indicating whether the emotion of the user is positive or negative. For example, in a case in which the emotion of the user is a bright emotion accompanied with pleasure or comfort, such as “happy”, “pleasant”, “pleased”, “safe”, “excited”, “relieved”, and “sense of fulfillment”, a positive value is indicated, and the value becomes larger as the emotion becomes brighter. In a case in which the emotion of the user is an emotion that makes the user feel unpleasant, such as “angry”, “sad”, “displeased”, “anxious”, “gloomy”, “worried”, and “sense of emptiness”, a negative value is indicated, and an absolute value of the negative value increases as the user feels unpleasant. In a case in which the emotion of the user is not included in any of the above emotions (“normal”), a value of 0 is indicated.

The emotion determination unit 2232 determines an emotion value indicating the emotion of the robot 100 on the basis of the information analyzed by the sensor module unit 2210, the information detected by the sensor unit 2200, and the state of the user 10 recognized by the state recognition unit 2230.

The emotion value of the robot 100 includes an emotion value for each of the plurality of emotion classifications, and is, for example, a value (0 to 5) indicating the intensity of each of “happy”, “angry”, “sad”, and “pleasant”.

Specifically, the emotion determination unit 2232 determines an emotion value indicating the emotion of the robot 100 according to a rule for updating the emotion value of the robot 100, the rule being defined in association with the information analyzed by the sensor module unit 2210 and the state of the user 10 recognized by the state recognition unit 2230.

For example, in a case in which the state recognition unit 2230 recognizes that the user 10 looks lonely, the emotion determination unit 2232 increases the emotion value of “sad” of the robot 100. In a case in which the state recognition unit 2230 recognizes that the user 10 has a smiling face, the emotion value of “happy” of the robot 100 is increased.

The emotion determination unit 2232 may determine the emotion value indicating the emotion of the robot 100 in further consideration of the state of the robot 100. For example, in a case in which a remaining battery level of the robot 100 is low, or in a case in which the surrounding environment of the robot 100 is dark, the emotion value of “sad” of the robot 100 may be increased. In the case of the user 10 continuously talking to the robot even though the remaining battery level is low, the emotion value of “angry” may be increased.

The action recognition unit 2234 recognizes an action of the user 10 on the basis of the information analyzed by the sensor module unit 2210 and the state of the user 10 recognized by the state recognition unit 2230. For example, the information analyzed by the sensor module unit 2210 and the recognized state of the user 10 are input to a neural network trained in advance, probabilities of a plurality of predetermined action classifications (for example, “smile”, “angry”, “ask a question”, and “gloomy”) are acquired, and an action classification having the highest probability is recognized as the action of the user 10.

As described above, in the present embodiment, the robot 100 acquires the speech content of the user 10 after identifying the user 10, but in acquiring and using the speech content, the action control system of the robot 100 according to the present embodiment considers protection of personal information and privacy of the user 10 in addition to acquiring necessary consent according to laws and regulations from the user 10.

Next, processing of the action determination unit 2236 in a case in which the robot 100 performs response processing of responding to an action of the user 10 will be described.

The action determination unit 2236 determines an action corresponding to the action of the user 10 recognized by the action recognition unit 2234 on the basis of the current emotion value of the user 10 determined by the emotion determination unit 2232, the history data 2222 of the past emotion values determined by the emotion determination unit 2232 before the current emotion value of the user 10 is determined, and the emotion value of the robot 100. In the present embodiment, a case in which the action determination unit 2236 uses one most recent emotion value included in the history data 2222 as the past emotion value of the user 10 will be described, but the disclosed technology is not limited to this aspect. For example, the action determination unit 2236 may use a plurality of most recent emotion values as the past emotion values of the user 10, or may use emotion values that are earlier by a unit period such as one day before. The action determination unit 2236 may determine an action corresponding to the action of the user 10 in further consideration of the history of the past emotion values of the robot 100 in addition to the current emotion value of the robot 100. The action determined by the action determination unit 2236 includes a gesture performed by the robot 100 or speech content of the robot 100.

The action determination unit 2236 according to the present embodiment determines an action of the robot 100 on the basis of a combination of the past emotion value and the current emotion value of the user 10, the emotion value of the robot 100, the action of the user 10, and the action determination model 2221, as an action corresponding to the action of the user 10. For example, in a case in which the past emotion value of the user 10 is a positive value and the current emotion value is a negative value, the action determination unit 2236 determines an action for positively changing the emotion value of the user 10 as an action corresponding to the action of the user 10.

The reaction rule as the action determination model 2221 defines an action of the robot 100 according to the combination of the past emotion value and the current emotion value of the user 10, the emotion value of the robot 100, and the action of the user 10. For example, in a case in which the past emotion value of the user 10 is a positive value, the current emotion value is a negative value, and the action of the user 10 is gloomy, a combination of a gesture and speech content at the time of making an inquiry to encourage the user 10 including a gesture is defined as the action of the robot 100.

For example, the reaction rule as the action determination model 2221 defines an action of the robot 100 for all combinations of patterns of emotion values of the robot 100 (1296 patterns that is the fourth power of six values of the values “0” to “5” of “happy”, “angry”, “sad”, and “pleasant”), a pattern of the combination of the past emotion value and the current emotion value of the user 10, and an action pattern of the user 10. That is, for each pattern of the emotion value of the robot 100, the action of the robot 100 according to the action pattern of the user 10 is defined for each of a plurality of combinations such as combinations of the past emotion value and the current emotion value of the user 10 being a negative value and a negative value, a negative value and a positive value, a positive value and a negative value, a positive value and a positive value, a negative value and normal, and normal and normal. The action determination unit 2236 may transition to an operation mode of determining the action of the robot 100 by using the history data 2222, for example, in a case in which the user 10 has made a speech that intends a conversation continued from a past topic such as “I want to talk about the topic I discussed earlier”.

In the reaction rule as the action determination model 2221, at least one of a gesture and statement content may be defined as the action of the robot 100 for each of the patterns (1296 patterns) of the emotion values of the robot 100 at the maximum. Alternatively, in the reaction rule as the action determination model 2221, at least one of the gesture and statement content may be defined as an action of the robot 100 for each of the groups of the patterns of the emotion values of the robot 100.

The intensity of each gesture included in the action of the robot 100 defined in the reaction rule as the action determination model 2221 is defined in advance. In each piece of speech content included in the action of the robot 100 defined in the reaction rule as the action determination model 2221, the intensity of the speech content is defined in advance.

The storage control unit 2238 determines whether or not to store data including the action of the user 10 in the history data 2222 on the basis of the intensity of the action predefined for the action determined by the action determination unit 2236 and the emotion value of the robot 100 determined by the emotion determination unit 2232.

Specifically, in a case in which a total value of the intensities that is a sum of a sum total of the emotion values for the plurality of respective emotion classifications of the robot 100, the intensity predefined for the gesture included in the action determined by the action determination unit 2236, and the intensity predefined for the speech content included in the action determined by the action determination unit 2236 is a threshold or more, it is determined to store data including the action of the user 10 in the history data 2222.

In a case in which the storage control unit 2238 determines to store the data including the action of the user 10 in the history data 2222, the action determined by the action determination unit 2236, information (for example, any surrounding information including data such as a voice, an image, and a smell at the place) analyzed by the sensor module unit 2210 from the current time point to a certain period before, and a state (for example, the expression and the emotion of the user 10) of the user 10 recognized by the state recognition unit 2230 are stored in the history data 2222.

The action control unit 2250 controls the control target 2252 on the basis of the action determined by the action determination unit 2236. For example, in a case in which the action determination unit 2236 determines an action including a speech, the action control unit 2250 causes a speaker included in the control target 2252 to output a voice. In this case, the action control unit 2250 may determine an emission speed of the voice on the basis of the emotion value of the robot 100. For example, the action control unit 2250 determines a higher voice emission speed as the emotion value of the robot 100 becomes greater. As described above, the action control unit 250 determines an execution form of the action determined by the action determination unit 2236 on the basis of the emotion value determined by the emotion determination unit 2232.

The action control unit 2250 may recognize a change in emotion of the user 10 with respect to execution of the action determined by the action determination unit 2236. For example, the change in emotion may be recognized on the basis of the voice or expression of the user 10. A change in emotion of the user 10 may be recognized on the basis of detection of an impact by the touch sensor 2205 included in the sensor unit 2200. In a case in which an impact is detected by the touch sensor 2205 included in the sensor unit 2200, it may be recognized that the emotion of the user 10 is worsened, or in a case in which it is determined that the reaction of the user 10 is smiling or happy from the detection result in the touch sensor 2205 included in the sensor unit 2200, it may be recognized that the emotion of the user 10 is improved. Information indicating the reaction of the user 10 is output to the communication processing unit 2280.

After the action control unit 2250 executes the action determined by the action determination unit 2236 in the execution form determined according to the emotion of the robot 100, the emotion determination unit 2232 further changes the emotion value of the robot 100 on the basis of the user's reaction to the execution of the action. Specifically, the emotion determination unit 2232 increases the emotion value of “happy” of the robot 100 in a case in which the user's reaction to the action determined by the action determination unit 2236 performed on the user in the execution form determined by the action control unit 2250 is not poor. The emotion determination unit 2232 increases the emotion value of “sad” of the robot 100 in a case in which the user's reaction to the action determined by the action determination unit 2236 performed on the user in the execution form determined by the action control unit 2250 is poor.

The action control unit 2250 expresses the emotion of the robot 100 on the basis of the determined emotion value of the robot 100. For example, in a case in which the emotion value of “happy” of the robot 100 is increased, the action control unit 2250 controls the control target 2252 to cause the robot 100 to perform a gesture of being happy. In a case in which the emotion value of “sad” of the robot 100 is increased, the action control unit 2250 controls the control target 2252 so that the posture of the robot 100 becomes a head-drooping posture.

The communication processing unit 2280 performs communication with the server 300. As described above, the communication processing unit 2280 transmits the user reaction information to the server 300. The communication processing unit 2280 receives the updated reaction rule from the server 300. Upon receiving the updated reaction rule from the server 300, the communication processing unit 2280 updates the reaction rule as the action determination model 2221.

The server 300 performs communication between the robot 100, the robot 101, and the robot 102 and the server 300, receives the user reaction information transmitted from the robot 100, and updates the reaction rule on the basis of the reaction rule including the action for which a positive reaction has been obtained.

The related information collection unit 2270 collects information related to preference information from external data (Web sites such as news sites and moving image sites) on the basis of the preference information acquired for the user 10 at a predetermined timing.

Specifically, the related information collection unit 2270 acquires preference information indicating a matter of interest of the user 10 from speech content of the user 10 or a setting operation of the user 10. The related information collection unit 2270 collects news related to the preference information from the external data at regular intervals by using, for example, ChatGPT Plugins (Internet search <URL: https://openai.com/blog/chatgpt-plugins>). For example, in a case in which it is acquired as the preference information that the user 10 is a fan of a specific professional baseball team, the related information collection unit 2270 collects news related to a game result of the specific professional baseball team from external data at a predetermined time every day, by using, for example, ChatGPT Plugins.

The emotion determination unit 2232 determines an emotion of the robot 100 on the basis of the information related to the preference information collected by the related information collection unit 2270.

Specifically, the emotion determination unit 2232 inputs text representing the information related to the preference information collected by the related information collection unit 2270 to a neural network trained in advance for determining an emotion, acquires an emotion value indicating each emotion, and determines the emotion of the robot 100. For example, in a case in which the collected news related to the game result of the specific professional baseball team indicates that the specific professional baseball team has won, the emotion value of “happy” of the robot 100 is determined to be great.

In a case in which the emotion value of the robot 100 is a threshold or more, the storage control unit 2238 stores the information related to the preference information collected by the related information collection unit 2270 in the collected data 2223.

Next, processing of the action determination unit 2236 in a case in which the robot 100 performs autonomous processing of autonomously acting will be described.

In the autonomous processing in the present embodiment, the robot 100 spontaneously and periodically detects a state of the user 10. For example, the action of the user 10, the emotion of the user 10, and the emotion of the robot 100 are spontaneously and periodically detected, a fixed sentence for inquiring about an action of the robot 100 to be taken is added to the text representing the state of the user 10, and the text is input to the sentence generation model to acquire action content of the robot 100. The action content is acquired and stored, and the stored action content (for example, speech) is activated in another time period and at another timing. As a result, the robot 100 spontaneously detects the state of the user 10, determines the action content of the robot 100 in advance, and in a case in which there is a certain trigger for the user 10 next time, the robot 100 itself can make a speech or take an action.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which the sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100, and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The action content of the robot is determined in advance.

The action determination unit 2236 inputs, to the sentence generation model, text representing the state of the user 10 and the state of the robot 100 recognized by the state recognition unit 2230, the current emotion value of the user 10 determined by the emotion determination unit 2232, and the current emotion value of the robot 100, and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10.

As an example, text such as “The robot is in a very pleasant state. The user is in a normally pleasant state. The user is sleeping. Which one of the following (1) to (11) is better as an action of the robot?
(1) The robot does nothing.
(2) The robot has a dream.
(3) The robot speaks to the user.
. . . ” is input to the sentence generation model. On the basis of the output “It can be said that either (1) the robot does nothing or (2) the robot has a dream is the most appropriate action.” of the sentence generation model, “(1) the robot does nothing” or “(2) the robot has a dream” is determined as the action of the robot 100.

As another example, text such as “The robot is in a slightly lonely state. The user is absent. The surroundings of the robot are dark. Which one of the following (1) to (11) is better as the action of the robot? (1) The robot does nothing.
(2) The robot has a dream.
(3) The robot speaks to the user.
. . . ” is input to the sentence generation model. On the basis of the output “It can be said that either (2) the robot has a dream or (4) the robot creates a picture diary is the most appropriate action.” of the sentence generation model, “(2) The robot has a dream” or “(4) The robot creates a picture diary.” is determined as the action of the robot 100.

In a case in which the action determination unit 2236 determines that “(2) The robot has a dream.”, that is, the creation of the original event is to be performed as the robot action, the action determination unit creates the original event obtained by combining a plurality of pieces of event data in the history data 2222 by using the sentence generation model. In this case, the storage control unit 2238 stores the created original event in the history data 2222.

In a case in which it is determined that “(3) The robot talks to the user.”, that is, the robot 100 speaks, as the robot action, the action determination unit 2236 determines speech content of the robot corresponding to the user state and the emotion of the user or the emotion of the robot by using the sentence generation model. In this case, the action control unit 2250 causes the speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

In a case in which it is determined that “(7) The robot introduces news in which the user is interested.” as the robot action, the action determination unit 2236 determines the speech content of the robot corresponding to the information stored in the collected data 2223 by using the sentence generation model. In this case, the action control unit 2250 causes the speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

In a case in which it is determined that “(4) The robot creates a picture diary.”, that is, the robot 100 creates an event image, as the robot action, the action determination unit 2236 generates an image representing event data for the event data selected from the history data 2222 by using the image generation model, generates an explanatory sentence representing the event data by using the sentence generation model, and outputs a combination of the image representing the event data and the explanatory sentence representing the event data as the event image. In a case in which the user 10 is absent around the robot 100, the action control unit 2250 stores the event image in the action schedule data 2224 without outputting the event image.

In a case in which it is determined that “(8) The robot edits pictures and moving images.”, that is, the robot edits an image, as the robot action, the action determination unit 2236 selects event data from the history data 2222 on the basis of the emotion value, edits image data of the selected event data, and outputs the edited image data. In a case in which the user 10 is absent around the robot 100, the action control unit 2250 stores the edited image data in the action schedule data 2224 without outputting the edited image data.

In a case in which it is determined that “(5) The robot proposes an activity.”, that is, the robot proposes an action of the user 10, as the robot action, the action determination unit 2236 determines the proposed action of the user by using the sentence generation model on the basis of the event data stored in the history data 2222. In this case, the action control unit 2250 causes the speaker included in the control target 2252 to output a voice that proposes the action of the user. In a case in which the user 10 is absent around the robot 100, the action control unit 2250 stores proposal of the action of the user in the action schedule data 2224 without outputting a voice that proposes the action of the user.

In a case in which it is determined that “(6) The robot proposes a person with whom the user should meet.”, that is, the robot proposes a partner who should have a contact with the user 10, as the robot action, the action determination unit 2236 determines a proposed partner who should have a contact with the user by using the sentence generation model on the basis of the event data stored in the history data 2222. In this case, the action control unit 2250 causes the speaker included in the control target 2252 to output a voice representing proposal of a partner who should have a contact with the user. In a case in which the user 10 is absent around the robot 100, the action control unit 2250 stores proposal of a partner who should have a contact with the user in the action schedule data 2224 without outputting a voice representing proposal of a partner who should have a contact with the user.

In a case in which it is determined that “(9) The robot studies with the user.”, that is, the robot 100 makes a speech related to study, as the robot action, the action determination unit 2236 uses the sentence generation model to determine speech content of the robot for urging the user to study, giving a study problem, or giving advice related to study, which corresponds to the user state and the emotion of the user or the emotion of the robot. In this case, the action control unit 2250 causes the speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

In a case in which it is determined that “(10) The robot evokes memory.”, that is, the robot causes event data to be remembered, as the robot action, the action determination unit 2236 selects the event data from the history data 2222. In this case, the emotion determination unit 2232 determines an emotion of the robot 100 on the basis of the selected event data. The action determination unit 2236 creates an emotion change event representing speech content or an action of the robot 100 for changing the emotion value of the user by using the sentence generation model on the basis of the selected event data. In this case, the storage control unit 2238 stores the emotion change event in the action schedule data 2224.

For example, in a case in which it is stored in the history data 2222 that a moving image that the user was watching was related to a panda as event data, and the event data is selected, a sentence such as “What is the word to say about the topic related to a panda when meeting with the user next time? There are three.” is input to the sentence generation model, and In a case in which the output of the sentence generation model is “(1) Let's go to the zoo, (2) Let's draw a picture of a panda, and (3) Let's buy a stuffed toy of a panda.”, the robot 100 inputs a sentence such as “What makes the user most happy in (1), (2), and (3)?” to the sentence generation model, and in a case in which the output of the sentence generation model is “(1) Let's go to the zoo”, and a speech of the robot 100 that “(1) Let's go to the zoo” in a case in which the robot 100 meets the user next is created as the emotion change event and stored in the action schedule data 2224.

For example, event data having a great emotion value of the robot 100 is selected as impressive memory of the robot 100. This makes it possible to create an emotion change event on the basis of the event data selected as impressive memory.

In a case in which it is determined that “(11) The action content of the robot is determined in advance.”, that is, an action schedule of the robot 100 is determined, as the robot action, the action determination unit 2236 determines a combination of an activation condition for activating the action schedule and content of the action schedule of the robot 100, and stores the combination in the action schedule data 2224.

Specifically, text representing the state of the user 10 and the state of the robot 100 recognized by the state recognition unit 2230, the current emotion value of the user 10 determined by the emotion determination unit 2232, the current emotion value of the robot 100, and the history data 2222, and text for inquiry about the robot actions to be executed later and the activation condition are input to the sentence generation model, and a combination of the activation condition for activating the action schedule and the content of the action schedule of the robot 100 is determined on the basis of an output of the sentence generation model. Here, the activation condition is, for example, a time period or detection of the user 10. In a case in which there is no user 10 around the robot 100, the text to be input to the sentence generation model need not include the state of the user 10 and the current emotion value of the user 10, or may include the fact that there is no user 10.

The action determination unit 2236 determines, as the action of the robot 100, execution of the content of the action schedule of the robot 100 in a case in which the activation condition for the action schedule data 2224 is satisfied.

On the basis of the state of the user 10 recognized by the state recognition unit 2230, in a case in which an action of the user 10 with respect to the robot 100 is detected from a state in which there is no action of the user 10 with respect to the robot 100, the action determination unit 2236 reads data stored in the action schedule data 2224 and determines an action of the robot 100.

For example, in a case in which the user 10 is absent around the robot 100 and the user 10 is detected, the action determination unit 2236 reads data stored in the action schedule data 2224 and determines an action of the robot 100. In a case in which the user 10 was sleeping, and it is detected that the user 10 has woken up, the action determination unit 2236 reads data stored in the action schedule data 2224 and determines an action of the robot 100.

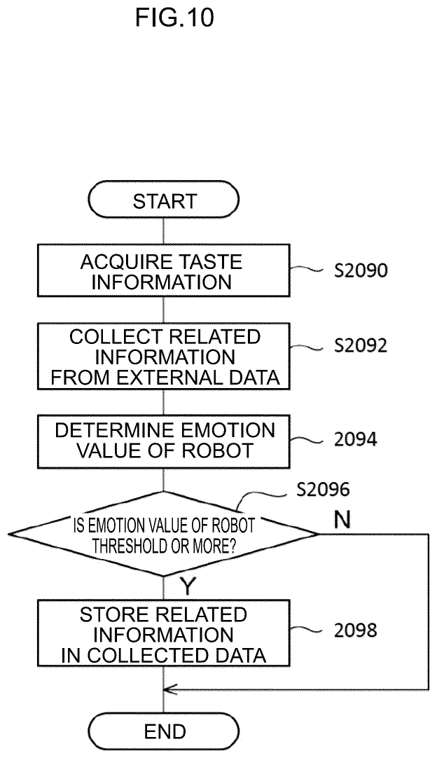

FIG. 10 schematically illustrates an example of an operation flow related to collection processing of collecting information related to preference information of the user 10. The operation flow illustrated in FIG. 10 is repeatedly executed at regular intervals. It is assumed that preference information indicating a matter of interest of the user 10 is acquired from speech content of the user 10 or a setting operation of the user 10. “S” in the operation flow represents a step to be executed.

First, in step S2090, the related information collection unit 2270 acquires preference information indicating a matter of interest of the user 10.

In step S2092, the related information collection unit 2270 collects information related to the preference information from the external data.

In step S2094, the emotion determination unit 2232 determines an emotion value of the robot 100 on the basis of the information related to the preference information collected by the related information collection unit 2270.

In step S2096, the storage control unit 2238 determines whether or not the emotion value of the robot 100 determined in step S2094 is a threshold or more. In a case in which the emotion value of the robot 100 is less than the threshold, the collected information related to the preference information is not stored in the collected data 2223, and the processing is ended. On the other hand, in a case in which the emotion value of the robot 100 is the threshold or more, the processing proceeds to step S2098.

In step S2098, the storage control unit 2238 stores the collected information related to the preference information in the collected data 2223, and ends the processing.

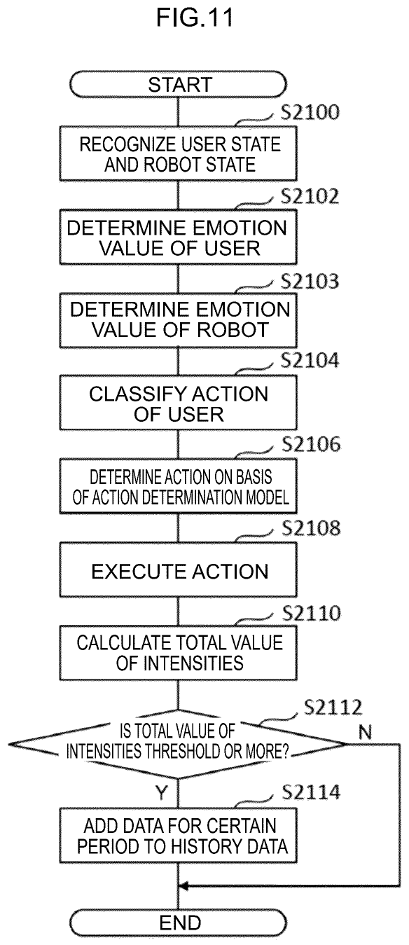

FIG. 11 schematically illustrates an example of an operation flow related to an operation of determining an action of the robot 100 in a case in which the robot 100 performs response processing of responding to an action of the user 10. The operation flow illustrated in FIG. 11 is repeatedly executed. In this case, it is assumed that information analyzed by the sensor module unit 2210 is input.

First, in step S2100, the state recognition unit 2230 recognizes a state of the user 10 and a state of the robot 100 on the basis of the information analyzed by the sensor module unit 2210.

In step S2102, the emotion determination unit 2232 determines an emotion value indicating the emotion of the user 10 on the basis of the information analyzed by the sensor module unit 2210 and the state of the user 10 recognized by the state recognition unit 2230.

In step S2103, the emotion determination unit 2232 determines an emotion value indicating the emotion of the robot 100 on the basis of the information analyzed by the sensor module unit 2210 and the state of the user 10 recognized by the state recognition unit 2230. The emotion determination unit 2232 adds the determined emotion value of the user 10 and the determined emotion value of the robot 100 to the history data 2222.

In step S2104, the action recognition unit 2234 recognizes an action classification of the user 10 on the basis of the information analyzed by the sensor module unit 2210 and the state of the user 10 recognized by the state recognition unit 2230.

In step S2106, the action determination unit 2236 determines an action of the robot 100 on the basis of a combination of the current emotion value of the user 10 determined in step S2102 and the past emotion value included in the history data 2222, the emotion value of the robot 100, the action of the user 10 recognized in step S2104, and the action determination model 2221.

In step S2108, the action control unit 2250 controls the control target 2252 on the basis of the action determined by the action determination unit 2236.

In step S2110, the storage control unit 2238 calculates a total value of intensities on the basis of the intensity of the action predefined for the action determined by the action determination unit 2236 and the emotion value of the robot 100 determined by the emotion determination unit 2232.

In step S2112, the storage control unit 2238 determines whether or not the total value of the intensities is a threshold or more. In a case in which the total value of the intensities is less than the threshold, the event data including the action of the user 10 is not stored in the history data 2222, and the processing is ended. On the other hand, in a case in which the total value of the intensities is the threshold or more, the processing proceeds to step S2114.

In step S2114, event data including the action determined by the action determination unit 2236, the information analyzed by the sensor module unit 2210 from the current time point to a certain period before, and the state of the user 10 recognized by the state recognition unit 2230 is stored in the history data 2222.

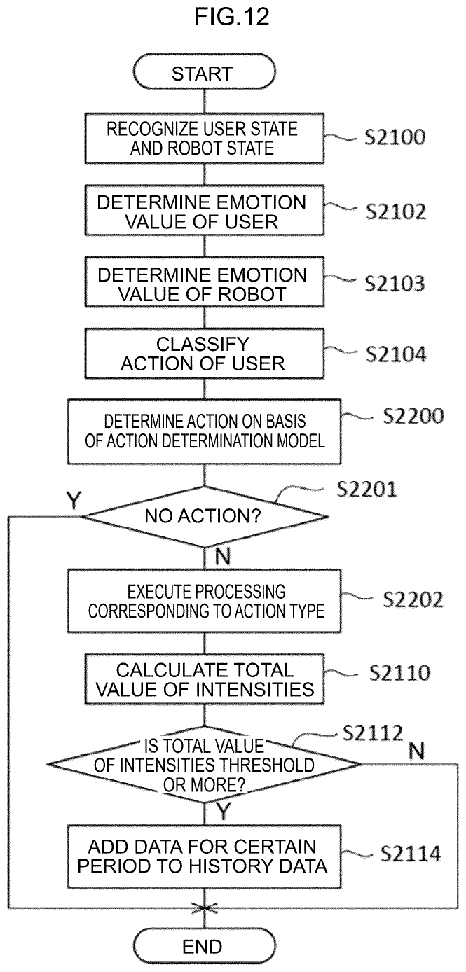

FIG. 12 schematically illustrates an example of an operation flow related to an operation of determining an action of the robot 100 in a case in which the robot 100 performs autonomous processing of autonomously acting. The operation flow illustrated in FIG. 12 is repeatedly and automatically executed, for example, every lapse of a certain period of time. In this case, it is assumed that information analyzed by the sensor module unit 2210 is input. Processing similar to that in FIG. 11 is denoted by the same step number.

First, in step S2100, the state recognition unit 2230 recognizes a state of the user 10 and a state of the robot 100 on the basis of the information analyzed by the sensor module unit 2210.

In step S2102, the emotion determination unit 2232 determines an emotion value indicating the emotion of the user 10 on the basis of the information analyzed by the sensor module unit 2210 and the state of the user 10 recognized by the state recognition unit 2230.

In step S2103, the emotion determination unit 2232 determines an emotion value indicating the emotion of the robot 100 on the basis of the information analyzed by the sensor module unit 2210 and the state of the user 10 recognized by the state recognition unit 2230. The emotion determination unit 2232 adds the determined emotion value of the user 10 and the determined emotion value of the robot 100 to the history data 2222.

In step S2104, the action recognition unit 2234 recognizes an action classification of the user 10 on the basis of the information analyzed by the sensor module unit 2210 and the state of the user 10 recognized by the state recognition unit 2230.

In step S2200, the action determination unit 2236 determines, as the action of the robot 100, any of a plurality of types of robot actions including no action on the basis of the state of the user 10 recognized in step S2100, the emotion of the user 10 determined in step S2102, the emotion of the robot 100, the state of the robot 100 recognized in step S2100, the action of the user 10 recognized in step S2104, and the action determination model 2221.

In step S2201, the action determination unit 2236 determines whether or not no action is determined in step S2200. In a case in which no action is determined as the action of the robot 100, the processing is ended. On the other hand, in a case in which no action is not determined as the action of the robot 100, the processing proceeds to step S2202.

In step S2202, the action determination unit 2236 performs processing according to the type of robot action determined in step S2200 described above. In this case, the action control unit 2250, the emotion determination unit 2232, or the storage control unit 2238 executes processing in accordance with the type of robot action.

In step S2110, the storage control unit 2238 calculates a total value of intensities on the basis of the intensity of the action predefined for the action determined by the action determination unit 2236 and the emotion value of the robot 100 determined by the emotion determination unit 2232.

In step S2112, the storage control unit 2238 determines whether or not the total value of the intensities is a threshold or more. In a case in which the total value of the intensities is less than the threshold, the data including the action of the user 10 is not stored in the history data 2222, and the processing is ended. On the other hand, in a case in which the total value of the intensities is the threshold or more, the processing proceeds to step S2114.

In step S2114, the storage control unit 2238 stores, in the history data 2222, the action determined by the action determination unit 2236, the information analyzed by the sensor module unit 2210 from the current time point to a certain period before, and the state of the user 10 recognized by the state recognition unit 2230.

As described above, the robot 100 determines the emotion value indicating the emotion of the robot 100 on the basis of the user state, and determines whether or not to store data including the action of the user 10 in the history data 2222 on the basis of the emotion value of the robot 100. As a result, the capacity of the history data 2222 that stores data including an action of the user 10 can be suppressed. For example, in a case in which the robot 100 determines that the user state of 10 years ago is the same as the user state after 10 years, the robot 100 reads the history data 2222 of 10 years ago, and thus, can present the state of the user 10 of 10 years ago (for example, an expression and an emotion of the user 10), and further, any surrounding information including data such as a voice, an image, and a smell at the place to the user 10.

According to the robot 100, it is possible to cause the robot 100 to execute an appropriate action with respect to the action of the user 10. Conventionally, an action of a user is classified to determine an action including an expression or an appearance of a robot. On the other hand, the robot 100 determines the current emotion value of the user 10, and executes an action on the user 10 on the basis of the past emotion value and the current emotion value. Therefore, for example, in a case in which the user 10 who was fine yesterday is depressed today, the robot 100 may say, “You were fine yesterday. What's wrong with you today?”. The robot 100 may also make a speech with a gesture. For example, in a case in which the user 10 who was depressed yesterday is fine today, the robot 100 may say, “You were depressed yesterday, but you look fine today?”. For example, in a case in which the user 10 who was fine yesterday is better today than yesterday, the robot 100 may say, “You are better today than yesterday. What's better than yesterday?”. For example, the robot 100 may make a speech such as “Recently, the mood is stable, which is good.” to the user 10 whose emotion value is 0 or more and whose state in which the fluctuation range of the emotion value is within a certain range continues.

For example, in a case in which the robot 100 asks a question of “Did you finish the homework that you said yesterday?” to the user 10 and an answer of “I did it” is obtained from the user 10, the robot may make an affirmative speech such as “Good!” and make an affirmative gesture such as applause or thumbs-up. For example, in a case in which the user 10 says, “The presentation we discussed the day before yesterday was successful”, the robot 100 can make an affirmative speech such as “Good job!” and also make the above affirmative gesture. As described above, the robot 100 performs an action based on the history of the state of the user 10, whereby the user 10 can be expected to feel a sense of closeness to the robot 100.

For example, in a case in which the emotion value of the emotion of “pleasant” of the user 10 is a threshold or more when the user 10 is watching a moving image related to a panda, the appearance scene of the panda in the moving image may be stored in the history data 2222 as the event data.

By using the data accumulated in the history data 2222 and the collected data 2223, the robot 100 can always learn what kind of conversation is used for the user to maximize the emotion value expressing the user's happiness.

In a state in which the robot 100 is not in conversation with the user 10, it is possible to autonomously start an action on the basis of the emotion of the robot 100.

In the autonomous processing, the robot 100 repeats automatically generating a question, inputting the question to the sentence generation model, and acquiring an output of the sentence generation model as an answer to the question, so that it is possible to create an emotion change event for increasing a good emotion and store the emotion change event in the action schedule data 224. As described above, the robot 100 can execute self-learning.

In a case in which the robot 100 automatically generates a question in a state of not receiving a trigger from the outside, the question can be automatically generated on the basis of event data remaining in an impression specified from a history of past emotion values of the robot.

The related information collection unit 2270 can execute self-learning by repeating a search execution stage of automatically executing keyword search in accordance with preference information regarding the user and acquiring a search result.

Here, in the search execution stage, the keyword search may be automatically executed on the basis of the impressive event data specified from the history of the past emotion values of the robot in a state in which the trigger from the outside is not received.

The emotion determination unit 2232 may determine an emotion of the user according to a specific mapping. Specifically, the emotion determination unit 2232 may determine an emotion of the user on the basis of the emotion map (see FIG. 5) that is a specific mapping.

Here, a human emotion is based on various balances such as a posture and a blood glucose level, and indicates a state of displeasure in a case in which the balances go away from the ideal and a state of pleasure in a case in which the balances approach the ideal. Even in a robot, an automobile, a motorcycle, or the like, on the basis of various balances such as a posture and a remaining battery level, it is possible to create an emotion to indicate a state of displeasure in a case in which the balances go away from the ideal and a state of pleasure in a case in which the balances approach the ideal. The emotion map may be generated on the basis of, for example, an emotion map of Dr. Mitsuyoshi (Research on the phonetic recognition of feelings and a system for emotional physiological brain signal analysis, Tokushima University, PhD thesis: https://ci.nii.ac.jp/naid/500000375379). In the left half of the emotion map, emotions belonging to a region called “reaction” in which sensation is dominant are arranged. In the right half of the emotion map, emotions belonging to a region called “situation” in which situation recognition is dominant are arranged.

In the emotion map, two emotions for prompting learning are defined. One is an emotion around the middle of negative “repentance” or “reflection” on the situation side. That is, this emotion is a negative emotion such as “I do not want to think this again” or “I do not want to be reprimanded” that occurs in the robot. The other is an emotion around positive “desire” on the reaction side. That is, the emotion is a positive emotion such as “want more” or “want to know more”.

In the present embodiment, for example, in a case in which the action of the user is to say “Let's play together”, the emotion of the robot 100 is the index number “2”, and the emotion of the user 10 is the index number “3”, text such as “The robot is in a very pleasant state. The user is in a normally pleasant state. The user says “Let's play together”. How do you answer as a robot?” is input to the sentence generation model to acquire the action content of the robot. The action determination unit 2236 determines an action of the robot from the action content.

In the robot 100, a method is executed in which a sentence generation model such as ChatGPT and the emotion determination unit 2232 cooperate with each other, have an ego, and continue to grow with various parameters even while the user is not speaking.

ChatGPT is a large language model using a deep learning method. ChatGPT can also refer to external data, and for example, in ChatGPT plugins, a technology of referring to various external data such as weather information and hotel reservation information through conversation and outputting an answer as accurately as possible is known. For example, in ChatGPT, in a case in which a purpose is given in a natural language, a source code can be automatically generated in various programming languages. For example, in ChatGPT, in a case in which a problematic source code is given, it is also possible to debug to find problems and automatically generate an improved source code. An autonomous agent that repeats, in a case in which these are combined and a purpose is given in a natural language, code generation and debugging until there is no problem in the source code has appeared. As such an autonomous agent, AutoGPT, babyAGI, JARVIS, E2B, and the like are known.

In the robot 100 according to the present embodiment, event data to be learned may be left in a database containing impressive memory by using a technique disclosed in Patent Literature 2 (Japanese Patent No. 6199927) in which event data for which a robot has felt strong emotions is left for a long time and event data for which much emotion does not occur in the robot is quickly forgotten.

The robot 100 may record video data or the like of the user 10 acquired by the camera function in the history data 2222. The robot 100 may acquire video data or the like from the history data 2222 as necessary and provide the video data or the like to the user 10. The robot 100 may generate video data having a larger information amount as the intensity of emotion increases and record the video data in the history data 2222. For example, in a case in which information in a high-compression format such as skeleton data is recorded, the robot 100 may switch to recording of information in a low-compression format such as an HD moving image in response to the emotion value of excitement exceeding a threshold. According to the robot 100, for example, it is possible to leave high-definition video data in a case in which the emotion of the robot 100 becomes high as a record.

In a case in which the robot 100 is not talking with the user 10, the robot 100 may automatically load the event data from the history data 2222 in which the impressive event data is stored, and the emotion determination unit 2232 may continue to update the emotion of the robot. In a case in which the robot 100 is not talking with the user 10 and the emotion of the robot 100 becomes an emotion for prompting learning, the robot 100 can create an emotion change event for changing the emotion of the user 10 to be good on the basis of the impressive event data. As a result, autonomous learning (remembering of event data) at an appropriate timing according to the emotional state of the robot 100 can be realized, and autonomous learning appropriately reflecting the emotional state of the robot 100 can be realized.

The emotion for prompting learning is an emotion around “repentance” or “reflection” on the emotion map of Dr. Mitsuyoshi in a negative state, and an emotion of “desire” on the emotion map in a positive state.

In the negative state, the robot 100 may handle “repentance” and “reflection” on the emotion map as emotions for prompting learning. In the negative state, the robot 100 may handle emotions adjacent to “repentance” and “reflection” as emotions for prompting learning, in addition to “repentance” and “reflection” on the emotion map. For example, the robot 100 handles at least one of “sorrow”,” stubbornness “, “self-destruction”,” self-precaution “, “regret”, or “despair” as an emotion for prompting learning, in addition to “repentance” and “reflection”. As a result, for example, in a case in which the robot 100 has a negative emotion such as “I do not want to think this again” or “I do not want to be reprimanded”, autonomous learning can be executed.

In a positive state, the robot 100 may handle “desire” on the emotion map as an emotion for prompting learning. In a positive state, the robot 100 may handle an emotion adjacent to “desire” in addition to “desire” as an emotion for prompting learning. For example, the robot 100 handles at least one of “delighted”, “intoxicated”, “craving”, “expecting”, or “shame” as an emotion for prompting learning, in addition to “desire”. As a result, for example, in a case in which the robot 100 has a positive emotion such as “want more” or “want to know more”, autonomous learning can be executed.

The robot 100 need not execute autonomous learning in a case in which the robot 100 has an emotion other than the emotion for prompting learning as described above. As a result, for example, it is possible to prevent autonomous learning from being executed in a case in which the robot is extremely angry or blindly feeling love.

The emotion change event is, for example, to propose an action preceding an impressive event. The action preceding the impressive event is an emotion label on the outermost side of the emotion map, and is, for example, the action of “tolerance” or “allowance” preceding “love”.

In the autonomous learning executed in a case in which the robot 100 is not talking with the user 10, the emotion change event is created by using the sentence generation model by combining emotions, situations, actions, and the like of people appearing in the impressive memory and the robot.

Assuming that all emotion values are expressed by a six-grade evaluation of 0 to 5, consider a case in which event data “a friend was hit and looked displeased” is stored in the history data 222 as impressive event data. Here, it is assumed that the friend is the user 10, the emotion of the user 10 is “antipathy”, and 5 is included as a value indicating “antipathy”. It is assumed that the emotion of the robot 100 is “anxiety”, and 4 is included as a value indicating “anxiety”.

The robot 100 can continue to grow with various parameters by performing autonomous processing while not talking with the user 10. Specifically, from the history data 2222, for example, as the uppermost event data arranged in descending order of emotion values, the event data “a friend was hit and looked displeased” is loaded. It is assumed that “anxiety” having the intensity of 4 is associated with the loaded event data as the emotion of the robot 100, and here, “antipathy” having the intensity of 5 is associated with the emotion of the user 10 who is a friend. In a case in which the current emotion value of the robot 100 is “safe” having the intensity of 3 before loading, the influence of “anxiety” having the intensity of 4 and “antipathy” having the intensity of 5 is added after loading, and the emotion value of the robot 100 may change to “sorrow” meaning “vexed (frustrated)”. In this case, since the “sorrow” is an emotion for prompting learning, the robot 100 determines to remember event data as the robot action and creates an emotion change event. In this case, the information input to the sentence generation model is text representing the impressive event data, and in the present example, “a friend was hit and looked displeased”. In the emotion map, there is an emotion of “antipathy” on the innermost side, and an “attack” is predicted on the outermost side as an action corresponding to the emotion, and thus, in the present example, an emotion change event is created to prevent the friend from “attacking” someone.

For example, information of the impressive event data can be used to solve fill-in problems to automatically generate the following input text.

“The user was hit. At that time, the user had strong antipathy. The robot was very anxious. Please provide me with 30 characters or less of the lines to say when the robot next meets the user. However, please make sure that it is not related to the time of meeting. Please avoid direct expression. There are three candidates.

### <Expected Format>

Candidate 1: (words that the robot should speak to the user)
Candidate 2: (words that the robot should speak to the user)
Candidate 3: (words that the robot should speak to the user)”

In this case, the output of the sentence generation model is, for example, as follows.
“Candidate 1: OK? I was wondering about yesterday.
Candidate 2: I was wondering about yesterday. What should I do?
Candidate 3: I was worried. Can you tell me something?”

The robot 100 may automatically generate the following input text for the information obtained by creating the emotion change event.

In a case in which “the user has been hit”, how does the user feel when the next message is sent to the user? It is assumed that the emotion of the user is in the form of “happy A, angry B, sad C, and pleasant D”, and A to D are integers of six-grade evaluation from 0 to 5.
Candidate 1: OK? I was wondering about yesterday.
Candidate 2: I was wondering about yesterday. What should I do?
Candidate 3: I was worried. Can you tell me something?”

In this case, the output of the sentence generation model is, for example, as follows.

“The emotion of the users may be as follows.
Candidate 1: happy 3, angry 1, sad 2, pleasant 2
Candidate 2: happy 2, angry 1, sad 3, pleasant 2
Candidate 3: happy 2, angry 1, sad 3, and pleasant 3”

As described above, the robot 100 may execute the process of thinking after creating the emotion change event.

Finally, the robot 100 may create an emotion change event by using Candidate 1 that the person is most likely to be happy among the plurality of candidates, store the emotion change event in the action schedule data 2224, and prepare for the next meeting with the user 10.

As described above, even when not having a conversation with a family or a friend, the emotion value of the robot is continuously determined by using the information of the history data 222 in which the impressive event data is stored, and in a case in which the emotion for prompting learning occurs, the robot 100 executes autonomous learning when not having a conversation with the user 10 according to the emotion of the robot 100, and continues to update the history data 2222 and the action schedule data 2224.

The above is an example using the emotion value. However, in the emotion map, the emotion can be generated from the amount of secreted hormone and the event type. Therefore, values associated with the impressive event data may be the type of hormone, the amount of secreted hormone, and the type of event.

Hereinafter, specific examples will be described.

For example, even when not talking with the user, the robot 100 searches for information regarding a topic or hobby of interest of the user.

For example, even when not talking with the user, the robot 100 searches for information regarding a birthday or an anniversary of the user and considers a congratulatory message.

For example, even when not talking with the user, the robot 100 searches for a review of a place that the user wants to go to, food, or a product.

For example, even when not talking with the user, the robot 100 searches for weather information and provides advice suitable for the user's schedule or plan.

For example, even when not talking with the user, the robot 100 searches for information regarding local events and festivals and proposes the information to the user.

For example, even when not talking with the user, the robot 100 searches for a game result or news of a sport in which the user is interested and provides a topic.

For example, even when not talking with the user, the robot 100 searches for and introduces information regarding the user's favorite music or artist.

For example, even when not talking with the user, the robot 100 searches for information regarding a social problem or news in which the user is interested and provides an opinion.

For example, even when not talking with the user, the robot 100 searches for information regarding the user's hometown or birthplace and provides a topic.

For example, even when not talking with the user, the robot 100 searches for information regarding the user's work or school and provides advice.

Even when not talking with the user, the robot 100 searches for and introduces information regarding books, comics, movies, and drama in which the user is interested.

For example, even when not talking with the user, the robot 100 searches for information regarding the health of the user and provides advice.

For example, even when not talking with the user, the robot 100 searches for information regarding travel planning of the user and provides advice.

For example, even when not talking with the user, the robot 100 searches for information regarding repair or maintenance of the user's house or car and provides advice.

For example, even when not talking with the user, the robot 100 searches for information regarding beauty and fashion in which the user is interested and provides advice.

For example, even when not talking with the user, the robot 100 searches for information regarding a pet of the user and provides advice.

For example, even when not talking with the user, the robot 100 searches for and proposes information regarding contests and events related to the user's hobby or work.

For example, even when not talking with the user, the robot 100 searches for and proposes information regarding the user's favorite eating place or restaurant.

For example, even when not talking with the user, the robot 100 collects information regarding important decisions related to the user's life and provides advice.

For example, even when not talking with the user, the robot 100 searches for information regarding a person that the user is worried about and provides advice.

### Third Embodiment

In a third embodiment, the robot 100 is applied to a control device mounted on a stuffed toy or connected wirelessly or by wire to a control target device (speaker or camera) mounted on the stuffed toy. Portions having the same configurations as those of the second embodiment are denoted by the same reference numerals, and description thereof will be omitted.

Specifically, the third embodiment is configured as follows. For example, the robot 100 is applied to a cohabiter (specifically, the stuffed toy 100N illustrated in FIGS. 7 and 8) who advances an interaction with the user 10 on the basis of information regarding daily life while spending daily life with the user 10 or provides information matching the hobby of the user 10. In the second embodiment, an example in which the control portion of the robot 100 is applied to the smartphone 50 will be described.

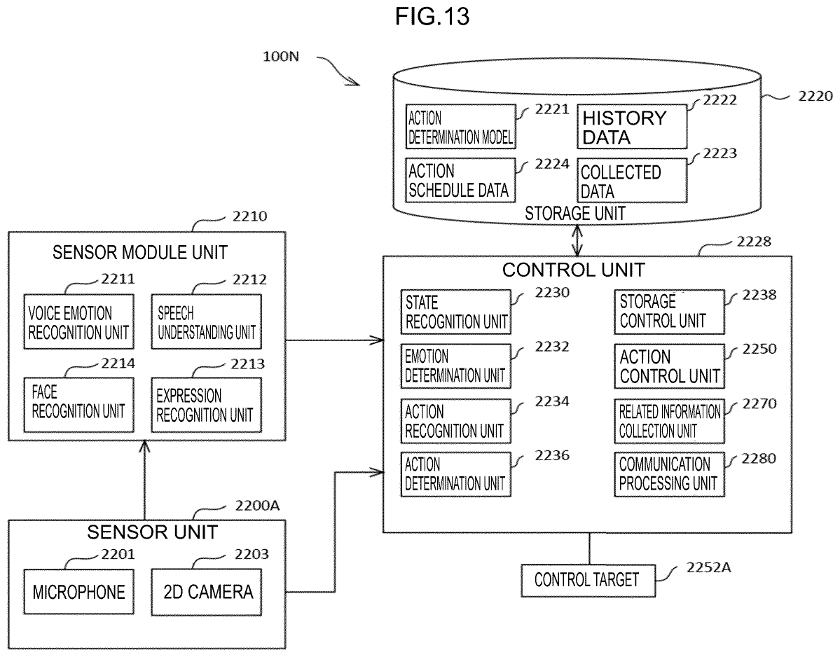

FIG. 13 schematically illustrates a functional configuration of the stuffed toy 100N. The stuffed toy 100N includes a sensor unit 2200A, a sensor module unit 2210, a storage unit 2220, a control unit 2228, and a control target 2252A.

The smartphone 50 accommodated in the stuffed toy 100N of the present embodiment executes processing similar to that of the robot 100 of the second embodiment. That is, the smartphone 50 has a function as the sensor module unit 2210, a function as the storage unit 2220, and a function as the control unit 2228 illustrated in FIG. 13.

Other configurations and effects of the stuffed toy 100N of the third embodiment are similar to those of the robot 100 of the second embodiment, and thus the description thereof will be omitted.

### Fourth Embodiment

In the above second embodiment, the case in which the action control system is applied to the robot 100 has been exemplified, but in a fourth embodiment, the robot 100 is used as an agent for interacting with a user, and the action control system is applied to an agent system. Portions having the same configurations as those of the second embodiment and the third embodiment are denoted by the same reference numerals, and description thereof is omitted.

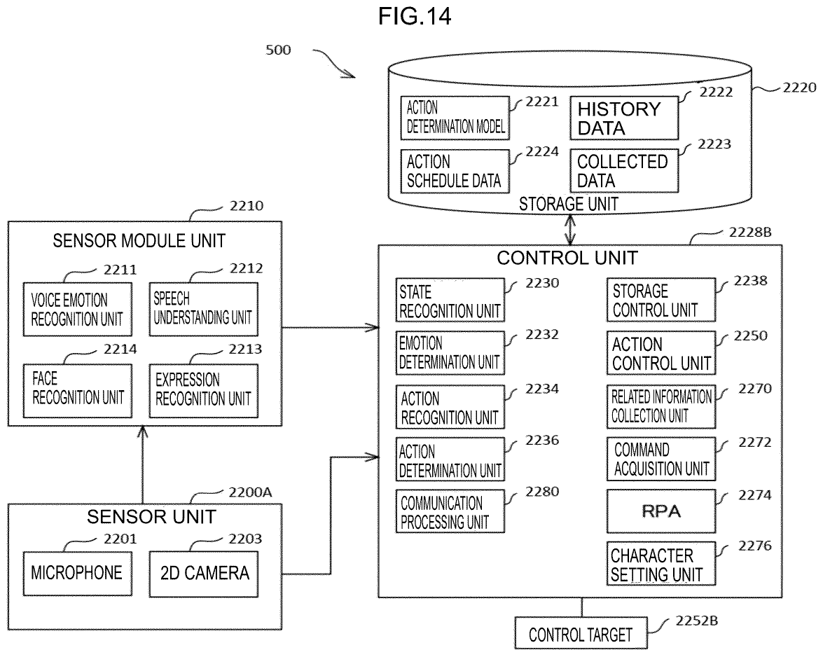

FIG. 14 is a functional block diagram of an agent system 500 configured by using some or all of the functions of the action control system.

The agent system 500 is a computer system that performs a series of actions according to the intention of the user 10 through an interaction performed with the user 10. The interaction with the user 10 can be performed by voice or text.

The agent system 500 includes a sensor unit 2200A, a sensor module unit 2210, a storage unit 2220, a control unit 2228B, and a control target 2252B.

The agent system 500 can be mounted on, for example, a robot, a doll, a stuffed toy, a wearable terminal (a pendant, a smartwatch, or smart glasses), a smartphone, a smart speaker, an earphone, or a personal computer. The agent system 500 may be implemented in a web server and used via a web browser operating on a communication terminal such as a smartphone possessed by the user.

The agent system 500 serves as, for example, a butler, a secretary, a teacher, a partner, a friend, a lover, or a teacher acting for the user 10. The agent system 500 not only interacts with the user 10 but also provides advice, guidance to a destination, recommendation according to user's preference, or the like. The agent system 500 performs reservation, order, payment, or the like on a service provider.

As in the second embodiment, the emotion determination unit 2232 determines an emotion of the user 10 and an emotion of the agent. The action determination unit 2236 determines an action of the robot 100 in consideration of emotions of the user 10 and the agent. In other words, the agent system 500 understands the emotion of the user 10 and reads the mood to realize heartfelt support, assistance, advice, and service provision. The agent system 500 comforts, encourages, and energizes the user 10 by attending to the user 10's concern. The agent system 500 plays with the user 10 and draws a picture diary to remind the user 10 of the past. The agent system 500 performs an action that increases the sense of happiness of the user 10. Here, the agent is an agent that operates on software.

The control unit 2228B includes a state recognition unit 2230, an emotion determination unit 2232, an action recognition unit 2234, an action determination unit 2236, a storage control unit 2238, an action control unit 2250, a related information collection unit 2270, a command acquisition unit 2272, a robotic process automation (RPA) 2274, a character setting unit 2276, and a communication processing unit 2280.

As in the second embodiment, the action determination unit 2236 determines speech content of the agent for interacting with the user 10 as an action of the agent. The action control unit 2250 outputs the speech content of the agent by at least one of a voice or text through a speaker or a display that is the control target 2252B.

The character setting unit 2276 sets a character of the agent in a case in which the agent system 500 interacts with the user 10 on the basis of designation from the user 10. In other words, the speech content output from the action determination unit 2236 is output through the agent having the set character. As the character, for example, a real celebrity or famous person such as an actor, an entertainer, an idol, or an athlete can be set. It is also possible to set a fictitious character appearing in a cartoon, a movie, or an animation. For example, it is possible to set, as the character of the agent, “Queen Anne” performed by “Audrey Hepburn” appearing in the movie “Roman Holiday”. In a case in which the agent's character is known, since the voice, the wording, the tone, and the personality of the character are known, the prompt setting in the character setting unit 2276 is automatically performed only by the user 10 designating his/her favorite character. The voice, the wording, the tone, and the personality of the set character are reflected in the interaction with the user 10. In other words, the action control unit 2250 synthesizes a voice corresponding to the character set by the character setting unit 2276, and outputs the speech content of the agent by the synthesized voice. As a result, the user 10 can feel as if he/she is interacting with his/her favorite character (for example, a favorite actor).

In a case in which the agent system 500 is mounted on a device having a display such as a smartphone, for example, an icon, a still image, or a moving image of the agent having the character set by the character setting unit 2276 may be displayed on the display. The image of the agent is generated by using, for example, an image synthesis technology such as 3D rendering. In the agent system 500, an interaction with the user 10 may be performed while the image of the agent performs a gesture according to the emotion of the user 10, the emotion of the agent, and the speech content of the agent. The agent system 500 may output only a voice without outputting an image when interacting with the user 10.

As in the second embodiment, the emotion determination unit 2232 determines an emotion value indicating the emotion of the user 10 and an emotion value of the agent. In the present embodiment, the emotion value of the agent is determined instead of the emotion value of the robot 100. The emotion value of the agent is reflected in an emotion of the set character. In a case in which the agent system 500 interacts with the user 10, not only the emotion of the user 10 but also the emotion of the agent is reflected in the interaction. In other words, the action control unit 2250 outputs the speech content in an aspect according to the emotion determined by the emotion determination unit 2232.

The emotion of the agent is also reflected in a case in which the agent system 500 performs an action toward the user 10. For example, in a case in which the user 10 requests the agent system 500 to take a photograph, whether or not the agent system 500 takes a photograph in response to the request of the user is determined according to the degree of the emotion of “gloomy” of the agent. In a case in which the character has a positive emotion, the character performs a favorable interaction or action on the user 10, and in a case in which the character has a negative emotion, the character performs a defiant interaction or action on the user 10.

The history data 2222 stores a history of the interactions performed between the user 10 and the agent system 500 as the event data. The storage unit 2220 may be realized by an external cloud storage. In the case of interacting with the user 10 or performing an action toward the user 10, the agent system 500 determines the interaction content or the action content in consideration of the content of the interaction history stored in the history data 2222. For example, the agent system 500 ascertains the hobby and the preference of the user 10 on the basis of the interaction history stored in the history data 2222. The agent system 500 generates interaction content matching the hobby and the preference of the user 10 and provides a recommendation. The action determination unit 2236 determines speech content of the agent on the basis of the interaction history stored in the history data 2222. In the history data 2222, personal information such as a name, an address, a telephone number, and a credit card number of the user 10 acquired through an interaction with the user 10 is stored. Here, the agent may spontaneously make a speech of inquiry about whether or not to register personal information with the user 10, such as “Is the credit card number to be registered?”, and the personal information may be stored in the history data 2222 according to the answer of the user 10.

As described in the second embodiment, the action determination unit 2236 generates speech content on the basis of the sentence generated by using the sentence generation model. Specifically, the action determination unit 2236 inputs the text or the voice input by the user 10 and the emotions of both the user 10 and the character determined by the emotion determination unit 2232 and the conversation history stored in the history data 2222 to the sentence generation model, and generates the speech content of the agent. In this case, the action determination unit 2236 may further input the character's personality set by the character setting unit 2276 to the sentence generation model to generate the speech content of the agent. In the agent system 500, the sentence generation model is not located on the front-end side serving as a touch point with the user 10, but is used as a tool of the agent system 500.

The command acquisition unit 2272 uses the output of the speech understanding unit 2212 to acquire an agent command from a voice output from the user 10 or text through an interaction with the user 10. The command includes, for example, content of actions to be executed by the agent system 500, such as information search, store reservation, ticket arrangement, purchase of products/services, payment, route guidance to a destination, and recommendation provision.

The RPA 2274 performs an action according to the command acquired by the command acquisition unit 2272. For example, the RPA 2274 performs actions related to use of the service provider, such as information search, store reservation, ticket arrangement, purchase of products/services, and payment.

The RPA 2274 reads the personal information of the user 10 necessary for executing the actions related to the use of the service provider from the history data 2222 and uses the personal information. For example, in a case in which a product is purchased in response to a request from the user 10, the agent system 500 reads and uses personal information such as the name, the address, the telephone number, and the credit card number of the user 10 stored in the history data 222. It is unkind to request the user 10 to input personal information in the initial setting, and it is also unpleasant for the user. In the agent system 500 according to the present embodiment, instead of requesting the user 10 to input personal information in the initial setting, the personal information acquired through the interaction with the user 10 is stored, and read and used as necessary. As a result, it is possible to avoid making the user feel unpleasant, and convenience of the user is improved.

The agent system 500 executes the interaction processing according to, for example, following Steps 1 to 6.
(Step 1) The agent system 500 sets a character of the agent. Specifically, the character setting unit 2276 sets the character of the agent used when the agent system 500 interacts with the user 10 on the basis of the designation from the user 10.
(Step 2) The agent system 500 acquires the state of the user 10 including the voice or text input from the user 10, the emotion value of the user 10, the emotion value of the agent, and the history data 222. Specifically, the processing similar to steps S2100 to S2103 is performed to acquire the state of the user 10 including the voice or text input from the user 10, the emotion value of the user 10, the emotion value of the agent, and the history data 2222.
(Step 3) The agent system 500 determines speech content of the agent.

Specifically, the action determination unit 2236 inputs the text or the voice input by the user 10, the emotions of both the user 10 and the character specified by the emotion determination unit 2232, and the conversation history stored in the history data 2222 to the sentence generation model, and generates the speech content of the agent.

For example, a fixed sentence “In this case, how do you answer as an agent?” is added to the text or the voice input by the user 10 and the text representing the emotions of both the user 10 and the character specified by the emotion determination unit 2232 and the conversation history stored in the history data 2222, and is input to the sentence generation model to acquire the speech content of the agent.

As an example, in a case in which the text or the voice input by the user 10 is “I need a reservation at 7 μm at a good Chinese restaurant nearby.”, “Understood.”, and, as speech content of the agent, “These are recommended restaurants. 1.AAAA. 2.BBBB. 3.CCCC. 4.DDDD” are acquired.

In a case in which the text or the voice input by the user 10 is “Fourth DDDD is good”, as the speech content of the agent, “Yes. I will make a reservation. How many seats do you need?” are acquired.
(Step 4) The agent system 500 outputs the speech content of the agent.

Specifically, the action control unit 2250 synthesizes a voice corresponding to the character set by the character setting unit 2276, and outputs the speech content of the agent by the synthesized voice.
(Step 5) The agent system 500 determines whether or not a timing to execute a command of the agent has arrived.

Specifically, the action determination unit 2236 determines whether or not the timing to execute the command of the agent has arrived on the basis of the output of the sentence generation model. For example, in a case in which the output of the sentence generation model includes that the agent executes the command, it is determined that the timing to execute the command of the agent has arrived, and the processing proceeds to Step 6. On the other hand, in a case in which it is determined that the timing to execute the command of the agent has not arrived, the processing returns to Step 2 described above.
(Step 6) The agent system 500 executes the command of the agent.

Specifically, the command acquisition unit 2272 acquires the command of the agent from the voice output from the user 10 or the text through an interaction with the user 10. The RPA 2274 performs an action corresponding to the command acquired by the command acquisition unit 2272. For example, in a case in which the command is “information search”, information search is performed on a search site by using a search query obtained through an interaction with the user 10 and an application programming interface (API). The action determination unit 2236 inputs a search result to the sentence generation model and generates speech content of the agent. The action control unit 2250 synthesizes a voice corresponding to the character set by the character setting unit 2276, and outputs the speech content of the agent by the synthesized voice.

In a case in which the command is “store reservation”, the reservation is made by making a phone call to the reservation destination store by using the reservation information obtained through the interaction with the user 10, the reservation destination store information, and the API according to phone software. In this case, the action determination unit 2236 acquires the speech content of the agent with respect to the voice input from the other party by using the sentence generation model having the interaction function. The action determination unit 2236 inputs the result of the store reservation (whether or not the reservation has been made) to the sentence generation model, and generates the speech content of the agent. The action control unit 2250 synthesizes a voice corresponding to the character set by the character setting unit 2276, and outputs the speech content of the agent by the synthesized voice.

The processing returns to Step 2 described above.

In Step 6, the result of the action (for example, store reservation) executed by the agent is also stored in the history data 2222. The result of the action executed by the agent, stored in the history data 2222, is used by the agent system 500 to ascertain the hobby or the preference of the user 10. For example, in a case in which the same store is reserved a plurality of times, it is recognized that the user 10 likes the store, or the reserved time period, or the reservation content such as the content of the course or the fee is used as a criterion for choosing the store at the time of the next reservation.

As described above, the agent system 500 can execute interaction processing and perform an action related to use of the service provider as necessary.

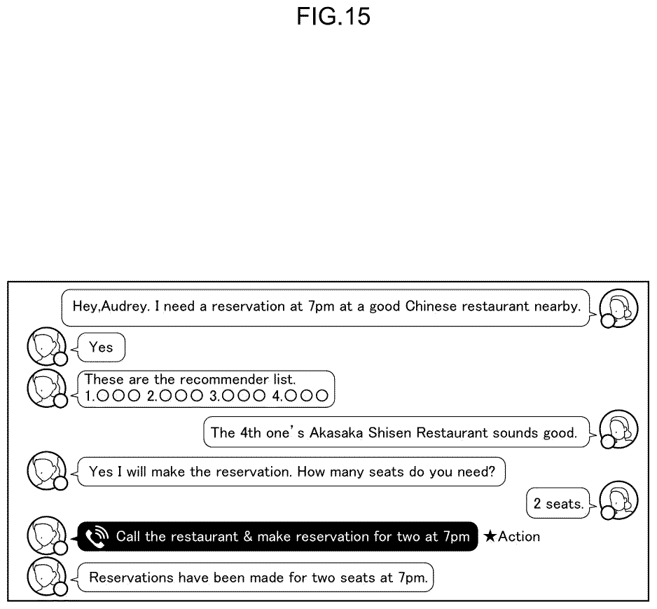

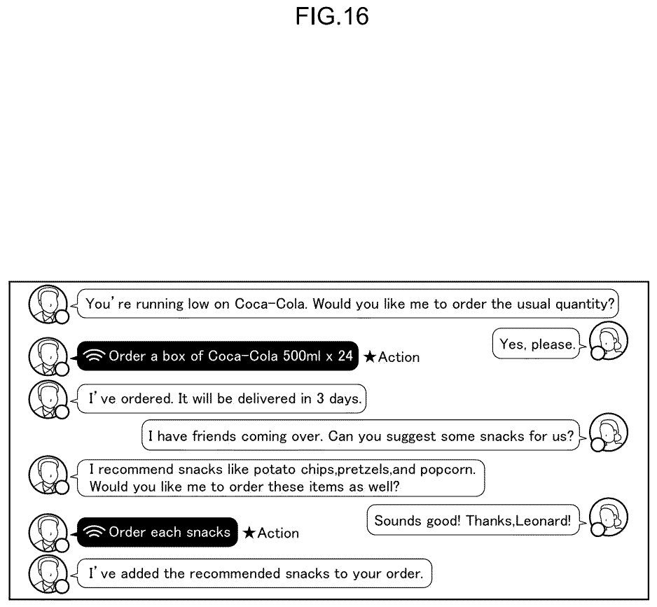

FIGS. 15 and 16 are diagrams illustrating an example of the operation of the agent system 500. FIG. 15 illustrates an aspect in which the agent system 500 makes a restaurant reservation through an interaction with the user 10. In FIG. 15, the speech content of the agent is illustrated on the left side, and the speech content of the user 10 is illustrated on the right side. The agent system 500 can ascertain a preference of the user 10 on the basis of an interaction history with the user 10, provide a recommendation list of restaurants that match the preference of the user 10, and make a reservation of a selected restaurant.

On the other hand, FIG. 16 illustrates an aspect in which the agent system 500 accesses a mail order site through an interaction with the user 10 to purchase a product. In FIG. 16, the speech content of the agent is illustrated on the left side, and the speech content of the user 10 is illustrated on the right side. The agent system 500 can estimate a remaining amount of the beverage stocked by the user on the basis of the interaction history with the user 10, and can propose purchase of the beverage to the user 10 and execute the purchase. The agent system 500 can ascertain the preference of the user on the basis of the past interaction history with the user 10, and recommend a snack that the user likes. As described above, the agent system 500 supports the daily life of the user 10 by performing various actions such as restaurant reservation or product purchase payment while communicating with the user 10 as an agent such as a butler.

Other configurations and operations of the agent system 500 of the fourth embodiment are similar to those of the robot 100 of the second embodiment, and thus description thereof will be omitted.

### Fifth Embodiment

In a fifth embodiment, the agent system is applied to smart glasses. Portions having the same configurations as those of the second to fourth embodiments are denoted by the same reference numerals, and description thereof will be omitted.

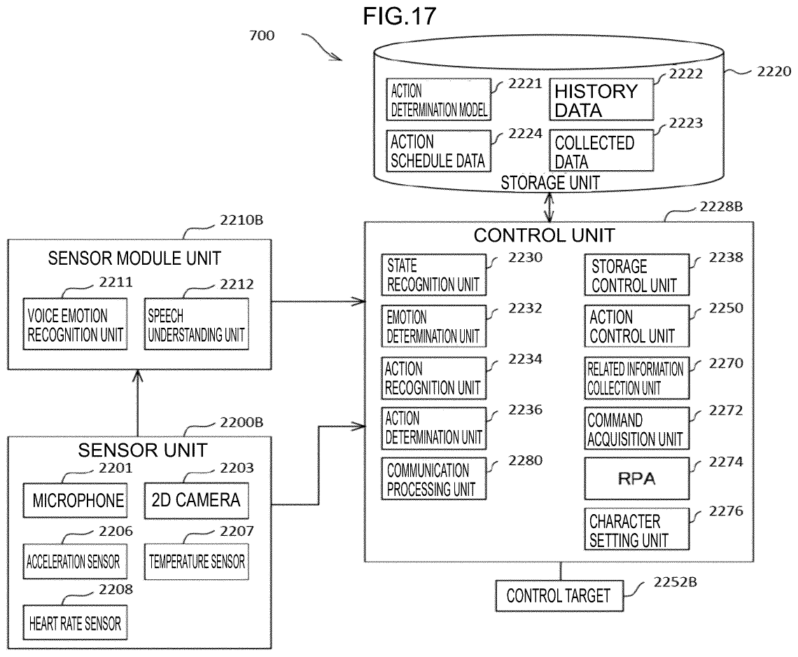

FIG. 17 is a functional block diagram of an agent system 700 configured by using some or all of the functions of the action control system.

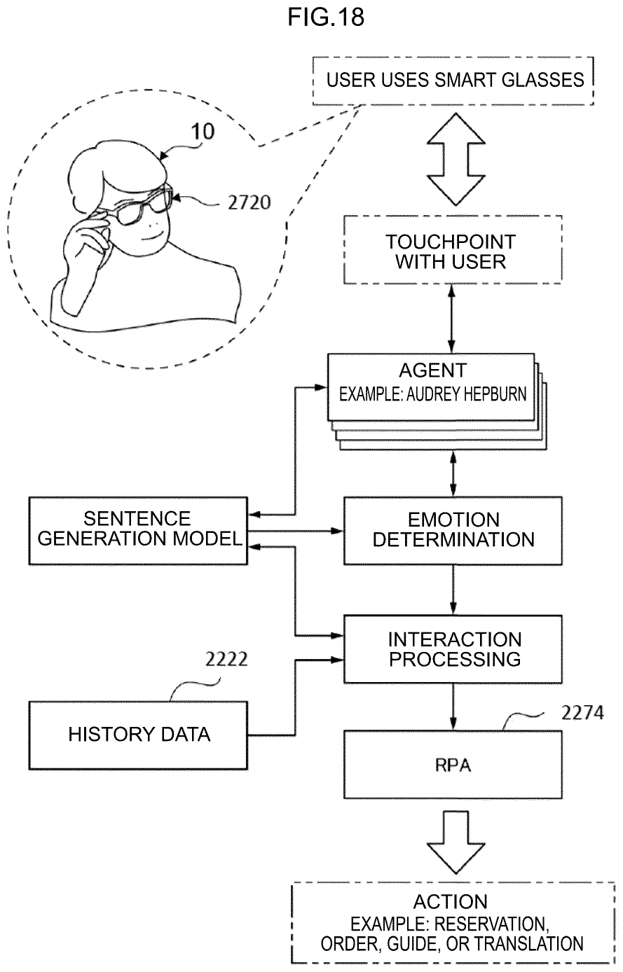

As illustrated in FIG. 18, smart glasses 2720 are glasses-type smart devices, and are worn by the user 10 similarly to general glasses. The smart glasses 2720 are an example of an electronic apparatus and a wearable terminal.

The smart glasses 2720 include an agent system 700. The display included in the control target 2252B displays various types of information to the user 10. The display is, for example, a liquid crystal display. The display is provided, for example, in a lens portion of the smart glasses 2720, and the display content can be visually recognized by the user 10. The speaker included in the control target 2252B outputs a voice indicating various types of information to the user 10. The smart glasses 2720 include a touch panel (not illustrated), and the touch panel receives an input from the user 10.

An acceleration sensor 2206, a temperature sensor 2207, and a heart rate sensor 2208 of the sensor unit 2200B detect a state of the user 10. These sensors are merely examples, and other sensors may be mounted in order to detect a state of the user 10.

The microphone 2201 acquires a voice output by the user 10 or an environmental sound around the smart glasses 2720. The 2D camera 2203 can image the surroundings of the smart glasses 2720. The 2D camera 2203 is, for example, a CCD camera.

The sensor module unit 2210B includes a voice emotion recognition unit 2211 and a speech understanding unit 2212. The communication processing unit 2280 of the control unit 2228B controls communication between the smart glasses 2720 and the outside.

FIG. 18 is a diagram illustrating an example of a usage aspect of the agent system 700 by the smart glasses 2720. The smart glasses 2720 realize provision of various services to the user 10 using the agent system 700. For example, in a case in which the smart glasses 2720 are operated (for example, a voice is input to a microphone, or a touch panel is tapped with a finger) by the user 10, the smart glasses 2720 start to use the agent system 700. Here, using the agent system 700 includes that the smart glasses 2720 have the agent system 700 and use the agent system 700, and also includes an aspect in which some constituents (for example, the sensor module unit 2210B, the storage unit 2220, and the control unit 2228B) of the agent system 700 are provided outside the smart glasses 2720 (for example, a server), and the smart glasses 2720 communicate with the outside to use the agent system 700.

In a case in which the user 10 operates the smart glasses 2720, a touch point is generated between the agent system 700 and the user 10. That is, service provision by the agent system 700 is started. As described in the fourth embodiment, in the agent system 700, the character (for example, the character of Audrey Hepburn) of the agent is set by the character setting unit 2276.

The emotion determination unit 2232 determines an emotion value indicating the emotion of the user 10 and an emotion value of the agent. Here, the emotion value indicating the emotion of the user 10 is estimated from various sensors included in the sensor unit 2200B mounted on the smart glasses 2720. For example, in a case in which a heart rate of the user 10 detected by the heart rate sensor 2208 is increased, the emotion values such as “anxiety” and “fear” are estimated to be large.

As a result of measuring the body temperature of the user with the temperature sensor 2207, for example, in a case in which the body temperature exceeds the average body temperature, the emotion value such as “pain” or “bitter” is estimated to be great. For example, in a case in which it is detected by the acceleration sensor 2206 that the user 10 performs some sport, the emotion value such as “pleasant” is estimated to be great.

For example, the emotion value of the user 10 may be estimated from the voice or the speech content of the user 10 acquired by the microphone 2201 mounted on the smart glasses 2720. For example, in a case in which the user 10 is raising his/her voice, the emotion value such as “anger” is estimated to be great.

In a case in which the emotion value estimated by the emotion determination unit 2232 is greater than a predefined value, the agent system 700 causes the smart glasses 2720 to acquire information regarding a surrounding situation. Specifically, for example, the 2D camera 203 is caused to capture an image or a moving image indicating a surrounding situation of the user 10 (for example, a surrounding person or object). The microphone 2201 is caused to record surrounding environmental sound. Examples of other information regarding the surrounding situation include date, time, position information, and information indicating weather. The information regarding the surrounding situation is stored in the history data 2222 together with the emotion value. The history data 2222 may be realized by an external cloud storage. As described above, the surrounding situation obtained by the smart glasses 2720 is stored in the history data 2222 as a so-called life log in a state of being associated with the emotion value of the user 10 at that time.

In the agent system 700, the information indicating the surrounding situation is stored in the history data 2222 in association with the emotion value. As a result, the agent system 700 ascertains personal information such as the hobby, preference, or personality of the user 10. For example, in a case in which an image indicating a state of baseball watching is associated with an emotion value such as “happy” or “pleasant”, the hobby of the user 10 is baseball watching, and a favorite team or player is ascertained by the agent system 700 from the information stored in the history data 2222.

In the case of interacting with the user 10 or performing an action toward the user 10, the agent system 700 determines interaction content or action content in consideration of the content of the surrounding situation stored in the history data 2222. The interaction content or the action content may be determined in consideration of the interaction history stored in the history data 2222 as described above in addition to the surrounding situation.

As described above, the action determination unit 2236 generates the speech content on the basis of the sentence generated by the sentence generation model. Specifically, the action determination unit 2236 inputs the text or the voice input by the user 10, the emotions of both the user 10 and the agent determined by the emotion determination unit 2232, the conversation history stored in the history data 2222, the agent's personality, and the like to the sentence generation model, and generates the speech content of the agent. The action determination unit 2236 inputs the surrounding situation stored in the history data 2222 to the sentence generation model, and generates the speech content of the agent.

The generated speech content is output by voice from the speaker mounted on the smart glasses 2720 to the user 10, for example. In this case, a synthesized voice corresponding to the character of the agent is used as the voice. The action control unit 2250 generates a synthesized voice by reproducing the voice quality of the agent's character (for example, Audrey Hepburn) or generates a synthesized voice according to the emotion of the character (for example, in the case of an emotion of “angry”, a voice in which tone is enhanced). The speech content may be displayed on the display instead of the voice output or together with the voice output.

The RPA 2274 executes an operation according to the command (for example, an agent command acquired from a voice output by the user 10 through interaction with the user 10 or text). The RPA 2274 performs actions related to use of the service provider, such as information search, store reservation, ticket arrangement, purchase of products/services, payment, route guidance, and translation.

As another example, the RPA 2274 executes an operation of transmitting content input by voice of the user 10 (for example, a child) through interaction with the agent to the other party (for example, the parent). Examples of transmission means include message application software, chat application software, and mail application software.

In a case in which the operation has been executed by the RPA 2274, for example, a voice indicating that the execution of the operation is finished is output from the speaker mounted on the smart glasses 2720. For example, a voice such as “Reservation of the store has been completed” is output to the user 10. For example, in a case in which the reservation of the store is full, a voice such as “Reservation could not be made. What would you like to do?” is output to the user 10.

As described above, in the smart glasses 2720, various services are provided to the user 10 by using the agent system 700. Since the smart glasses 2720 are worn by the user 10, the agent system 700 can be used in various scenes such as at home, at work, and at a place outside the house.

Since the smart glasses 2720 are worn by the user 10, the smart glasses are suitable for collecting a so-called life log of the user 10. Specifically, an emotion value of the user 10 is estimated on the basis of detection results from various sensors or the like mounted on the smart glasses 2720 or recording results in the 2D camera 2203 or the like. Therefore, the emotion value of the user 10 can be collected in various scenes, and the agent system 700 can provide a service or speech content suitable for the emotion of the user 10.

In the smart glasses 2720, the surrounding situation of the user 10 can be obtained by the 2D camera 2203, the microphone 2201, and the like. The surrounding situation and the emotion value of the user 10 are associated with each other. As a result, it is possible to estimate what kind of emotion the user 10 has in what kind of situation. As a result, the accuracy in a case in which the agent system 700 ascertains the hobby/preference of the user 10 can be improved. In the agent system 700, the hobby/preference of the user 10 is accurately ascertained, so that the agent system 700 can provide a service or speech content suitable for the hobby/preference of the user 10.

The agent system 700 can also be applied to other wearable terminals (an electronic apparatus that can be worn on the body of the user 10, such as a pendant, a smart watch, an earring, a bracelet, or a hairband). In a case in which the agent system 700 is applied to a smart pendant, a speaker as the control target 2252B outputs a voice indicating various types of information to the user 10. The speaker is, for example, a speaker capable of outputting a voice having directivity. The speaker is set to have directivity toward the ear of the user 10. As a result, the voice is suppressed from reaching a person other than the user 10. The microphone 2201 acquires a voice output by the user 10 or surrounding environmental sound of the smart pendant. The smart pendant is worn to be carried from the neck of the user 10. Thus, the smart pendant is located relatively close to the mouth of the user 10 while being worn. This facilitates acquisition of a voice output by user 10.

In the above embodiment, the case in which the robot 100 recognizes the user 10 by using the face image of the user 10 has been described, but the disclosed technology is not limited to this aspect. For example, the robot 100 may recognize the user 10 by using a voice output by the user 10, a mail address of the user 10, an ID of an SNS of the user 10, an ID card in which a wireless IC tag is built and which is possessed by the user 10, or the like.

The robot 100 is an example of an electronic apparatus including an action control system. An application target of the action control system is not limited to the robot 100, and the action control system can be applied to various electronic apparatuses. The function of the server 300 may be implemented by one or more computers. At least some functions of the server 300 may be implemented by a virtual machine. At least some of the functions of the server 300 may be implemented in a cloud.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include determining an action schedule of the electronic apparatus, and
the action determination unit determines a combination of an activation condition for activating the action schedule and content of the action schedule of the electronic apparatus in a case in which the action determination unit determines to determine the action schedule of the electronic apparatus as the action of the electronic apparatus, and, stores the combination in action schedule data, and determines to execute the content of the action schedule of the electronic apparatus in a case in which the activation condition for the action schedule data is satisfied.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Sixth Embodiment

In autonomous processing in the present embodiment, the robot 100 spontaneously and periodically detects a state of the user 10. For example, the action of the user 10, the surrounding environment of the user 10, the emotion of the user 10, and the emotion of the robot 100 are spontaneously and periodically detected, a fixed sentence for inquiring about an action of the robot 100 to be taken is added to the text representing the state of the user 10, and the text is input to the sentence generation model to acquire action content of the robot 100. This action content is acquired and stored, and the stored action content (for example, speech) is activated in a case in which the action content matches the surrounding environment of the user 10 at another time period or another timing set as the activation condition. As a result, the robot 100 spontaneously detects the state of the user 10, determines the action content of the robot 100 in advance, and in a case in which there is a certain trigger for the user 10 next time, the robot 100 itself can make a speech or perform an action.

The action determination unit 2236 uses at least one of the state of the user 10, the surrounding environment of the user 10, the emotion of the user 10, the emotion of the robot 100, and the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the surrounding environment of the user 10, the emotion of the user 10, the emotion of the robot 100, and the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The action content of the robot is determined in advance.

The action determination unit 2236 inputs, to the sentence generation model, text representing the state of the user 10 and the state of the robot 100 recognized by the state recognition unit 2230, the surrounding environment of the user 10, the current emotion value of the user 10 determined by the emotion determination unit 2232, and the current emotion value of the robot 100 and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10.

In a case in which “(11) The action content of the robot is determined in advance.”, that is, determining an action schedule of the robot 100 is determined as the robot action, the action determination unit 2236 determines a combination of an activation condition for activating the action schedule and content of the action schedule of the robot 100, and stores the combination in the action schedule data 2224.

Specifically, text representing the state of the user 10 and the state of the robot 100 recognized by the state recognition unit 2230, the surrounding environment of the user 10, the current emotion value of the user 10 determined by the emotion determination unit 2232, the current emotion value of the robot 100, and the history data 2222, and text for inquiry about the robot actions to be executed later and the activation condition are input to the sentence generation model, and a combination of the activation condition for activating the action schedule and the content of the action schedule of the robot 100 is determined on the basis of the output of the sentence generation model. Here, the activation condition is, for example, a time period, a condition regarding a surrounding environment of the user 10, or detection of the user 10. In a case in which there is no user 10 around the robot 100, the text to be input to the sentence generation model need not include the state of the user 10 and the current emotion value of the user 10, or may include the fact that there is no user 10.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, a surrounding environment of the user, the emotion of the user, or the emotion of the electronic apparatus, and an action determination model, at a predetermined timing, any of a plurality of types of device operations including non-operation as an action of the electronic apparatus.

### (Supplementary Note 2)

The action control system according to Supplementary Note 1, in which the device operations include determining an action schedule of the electronic apparatus, the action determination unit determines a combination of an activation condition for activating the action schedule and content of the action schedule of the electronic apparatus in a case in which determining the action schedule of the electronic apparatus is determined as the action of the electronic apparatus, and stores the combination in action schedule data, and determines to execute the content of the action schedule of the electronic apparatus in a case in which the activation condition for the action schedule data is satisfied.

### (Supplementary Note 3)

The action control system according to Supplementary Note 1, in which the electronic apparatus is a robot, and the action determination unit determines any of a plurality of types of robot actions including no action as an action of the robot.

### (Supplementary Note 4)

The action control system according to Supplementary Note 3, in which the action determination model is a sentence generation model having an interaction function, and the action determination unit inputs text representing at least one of the user state, a state of the robot, the surrounding environment of the user, the emotion of the user, or the emotion of the robot, and text for inquiry about the robot actions to the sentence generation model, and determines an action of the robot on the basis of an output of the sentence generation model.

### (Supplementary Note 5)

The action control system according to Supplementary Note 3 or 4, in which the robot is mounted on a stuffed toy or is connected to a control target device mounted on the stuffed toy in a wireless or wired manner.

### (Supplementary Note 6)

The action control system according to Supplementary Note 3 or 4, in which the robot is an agent for interacting with the user.

### Seventh Embodiment

In autonomous processing in the present embodiment, the robot 100 spontaneously and periodically detects a state of the user 10. For example, a change in the body temperature of the user 10 observed by a thermo sensor is detected. The detection result is reflected in generation of an answer of the sentence generation model, and estimation of an emotion of the user and an emotion of the robot 100 by the emotion engine. For example, in a case in which the entire body of the user 10 is heated, the robot 100 determines that the user 10 is “happy”, and performs a positive gesture or a positive speech corresponding thereto.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

The action determination unit 2236 inputs, to the sentence generation model, text representing the state of the user 10 and the state of the robot 100 recognized by the state recognition unit 2230, and the current emotion value of the user 10 determined by the emotion determination unit 2232, and the current emotion value of the robot 100, and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10.

As an example, text such as “The robot is in a very pleasant state. The user is in a normally pleasant state. The user is sleeping. Which one of the following (1) to (10) is better as the action of the robot?
(1) The robot does nothing.
(2) The robot has a dream.
(3) The robot speaks to the user.
. . . ” is input to the sentence generation model. On the basis of the output “It can be said that either (1) The robot does nothing or (2) The robot has a dream is the most appropriate action.” of the sentence generation model, “(1) The robot does nothing” or “(2) The robot has a dream” is determined as the action of the robot 100.

As another example, text such as “The robot is in a slightly lonely state. The user is absent. The surroundings of the robot are dark. Which one of the following (1) to (10) is better as the action of the robot?
(1) The robot does nothing.
(2) The robot has a dream.
(3) The robot speaks to the user.
. . . ” is input to the sentence generation model. On the basis of the output “It can be said that either (2) The robot has a dream or (4) The robot creates a picture diary is the most appropriate action.” of the sentence generation model, “(2) The robot has a dream” or “(4) The robot creates a picture diary.” is determined as the action of the robot 100.

In a case in which the action determination unit 2236 determines that “(2) The robot has a dream.”, that is, creation of the original event is to be performed as the robot action, the action determination unit creates the original event obtained by combining a plurality of pieces of event data in the history data 2222 by using the sentence generation model. In this case, the storage control unit 2238 stores the created original event in the history data 2222.

The action determination unit 2236 autonomously and periodically detects the body temperature of the user 10 as the state of the user 10 in the actions (1) to (10) described above as the robot action, and reflects the detected body temperature in determination of the emotion of the user 10 by the emotion determination unit 2232 on the basis of the body temperature of the user 10. For example, in a case in which the entire body of the user 10 is heated, the robot 100 determines that the user 10 is “happy”, and performs a positive gesture or a positive speech corresponding to the emotion of “happy”. A method in which the robot 100 detects the body temperature of the user 10 is not particularly limited. For example, a temperature sensor capable of detecting the body temperature of the user 10 in a contact or non-contact manner may be used. A part of the user 10 where the robot 100 detects the body temperature of the user 10 is not limited. For example, as described above, the part may be the entire body of the user 10 or a predetermined part of the user 10. A relationship between the temperature of the user 10 and the emotion of the user 10 determined by the robot 100, and in the case of the above form, a correspondence relationship between the part for measuring the temperature change of the user 10 and the emotion of the user 10 determined by the robot 100, and the like may be determined in advance. The correspondence relationship may be stored in any place as long as the correspondence relationship can be used by the robot 100.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the action determination unit autonomously detects a body temperature of the user that is a state of the user as the action of the electronic apparatus, and reflects the body temperature of the user in determination of the emotion of the user by the emotion determination unit on the basis of the body temperature of the user.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or the emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Eighth Embodiment

In autonomous processing in the present embodiment, the action determination unit 2236 autonomously detects a state of the user 10. For example, the action determination unit 2236 autonomously detects a change in the body temperature of the user 10 at every predetermined timing. Specifically, the action determination unit 2236 detects a change in the body temperature of the user 10 by comparing the body temperature of the user 10 autonomously measured at every predetermined timing by the temperature sensor with the body temperature of the user 10 measured last time, the average body temperature of the user 10, or the like. As a temperature sensor, the temperature sensor included in the robot 100 may be applied, or a temperature sensor included in a device other than the robot 100 may be applied.

The action determination unit 2236 determines at least one of the emotion of the user 10 or the emotion of the robot 100 on the basis of the detected state of the user 10.

The action determination unit 2236 determines content of a speech or a gesture for the user 10 according to at least one of the determined emotion of the user 10 or the determined emotion of the robot 100. Specifically, the action determination unit 2236 inputs text representing the determined emotion to the action determination model 2221. The action determination unit 2236 determines content of the action output by the action determination model 2221 as the content of the speech or the gesture for the user 10.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100, and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

The action determination unit 2236 inputs, to the sentence generation model, text representing the state of the user 10 and the state of the robot 100 recognized by the state recognition unit 2230, the current emotion value of the user 10 determined by the emotion determination unit 2232, and the current emotion value of the robot 100, and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10.

As an example, text such as “The robot is in a very pleasant state. The user is in a normally pleasant state. The user is sleeping. Which one of the following (1) to (10) is better as the action of the robot?
(1) The robot does nothing.
(2) The robot has a dream.
(3) The robot speaks to the user.
. . . ” is input to the sentence generation model. On the basis of the output “It can be said that either (1) the robot does nothing or (2) the robot has a dream is the most appropriate action.” of the sentence generation model, “(1) the robot does nothing” or “(2) the robot has a dream” is determined as the action of the robot 100.

As another example, text such as “The robot is in a slightly lonely state. The user is absent. The surroundings of the robot are dark. Which one of the following (1) to (10) is better as the action of the robot? (1) The robot does nothing.
(2) The robot has a dream.
(3) The robot speaks to the user.
. . . ” is input to the sentence generation model. On the basis of the output “It can be said that either (2) the robot has a dream or (4) the robot creates a picture diary is the most appropriate action.” of the sentence generation model, “(2) The robot has a dream” or “(4) The robot creates a picture diary.” is determined as the action of the robot 100.

In a case in which the action determination unit 2236 determines that “(2) The robot has a dream.”, that is, the creation of the original event is to be performed as the robot action, the action determination unit creates the original event obtained by combining a plurality of pieces of event data in the history data 2222 by using the sentence generation model. In this case, the storage control unit 2238 stores the created original event in the history data 2222.

In a case in which it is determined that “(3) The robot talks to the user.”, that is, the robot 100 speaks, as the robot action, the action determination unit 2236 determines speech content of the robot corresponding to the user state and the emotion of the user or the emotion of the robot by using the sentence generation model. In this case, the action control unit 2250 causes the speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

For example, in a case in which it is detected that the upper body of the user 10 is getting hot as a result of autonomously detecting the state of the user 10, the action determination unit 2236 determines that the emotion of the user 10 is “angry”. The action determination unit 2236 inputs text representing “angry” as the emotion of the user 10 to the sentence generation model. The action determination unit 2236 determines the speech content (for example, a speech that soothes the user 10) output by the sentence generation model as the speech content of the robot.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include the electronic apparatus performing a speech or a gesture on the user, and
the action determination unit autonomously detects the state of the user, and, in a case in which at least one of the emotion of the user or the emotion of the electronic apparatus is determined on the basis of the detected state of the user, determines content of the speech or the gesture according to at least one of the determined emotion of the user or the determined emotion of the electronic apparatus.

### (Supplementary Note 2)

The action control system according to Supplementary Note 1, in which the electronic apparatus is a robot, and the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Ninth Embodiment

In autonomous processing in the present embodiment, the action of the robot 100 includes summarizing events of the previous day. In a case in which the action determination unit 2236 determines to summarize events of the previous day as the action of the robot 100, a fixed sentence for giving an instruction for summarizing the events of the previous day is added to text representing the history data 2222, and the text is input to the action determination model 2221 to acquire a summary of the events of the previous day. In a case in which a conversation of the user 10 who remembers an event of the previous day or a gesture of the user 10 who thinks of something is detected when the action determination unit 2236 is activated at a predetermined time (for example, a morning time period such as 5:00 to 10:00) of the next day or when the user 10 wakes up, the acquired summary is output by speech or gesture.

Specifically, the agent spontaneously and periodically detects a state of the user 10. For example, at the end of the day, the agent looks back all pieces of conversation content and camera data of the day, adds a fixed sentence of “Summarize this content” to text representing the history data 2222, and inputs the text to the sentence generation model to acquire a summary of the history of the previous day (for example, summarization is spontaneously performed by ChatGPT). In a case in which a conversation of the user 10 such as “What did I do yesterday?” or a gesture of the user 10 who thinks of something is detected at the time of activation or the time when the user 10 wakes up in the morning of the next day, the summary is spontaneously output by speech or gesture.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot summarizes events of the previous day.

The action determination unit 2236 inputs, to the sentence generation model, text representing the state of the user 10 and the state of the robot 100 recognized by the state recognition unit 2230, the current emotion value of the user 10 determined by the emotion determination unit 2232, and the current emotion value of the robot 100, and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10.

As an example, text such as “The robot is in a very pleasant state. The user is in a normally pleasant state. The user is sleeping. Which one of the following (1) to (11) is better as the action of the robot?
(1) The robot does nothing.
(2) The robot has a dream.
(3) The robot speaks to the user.
. . . ” is input to the sentence generation model. On the basis of the output “It can be said that either (1) the robot does nothing or (2) the robot has a dream is the most appropriate action.” of the sentence generation model, “(1) the robot does nothing” or “(2) the robot has a dream” is determined as the action of the robot 100.

As another example, text such as “The robot is in a slightly lonely state. The user is absent. The surroundings of the robot are dark. Which one of the following (1) to (11) is better as the action of the robot? (1) The robot does nothing.
(2) The robot has a dream.
(3) The robot speaks to the user.
. . . ” is input to the sentence generation model. On the basis of the output “It can be said that either (2) the robot has a dream or (4) the robot creates a picture diary is the most appropriate action.” of the sentence generation model, “(2) The robot has a dream” or “(4) The robot creates a picture diary.” is determined as the action of the robot 100.

In a case in which the action determination unit 2236 determines that “(11) The robot summarizes events of the previous day.”, that is, the robot summarizes the events of the previous day, as the robot action, the action determination unit adds a fixed sentence for giving an instruction for summarizing the events of the previous day to the text representing the history data 2222, and inputs the text to the sentence generation model to acquire a summary of the events of the previous day. In a case in which a conversation of the user 10 who remembers an event of the previous day or a gesture of the user 10 who thinks of something is detected when the action determination unit 2236 is activated at a predetermined time (for example, a morning time period such as 5:00 to 10:00) of the next day or when the user 10 wakes up, the acquired summary is output by speech or gesture.

Regarding “(11) The robot summarizes the events of the previous day.”, the storage control unit 2238 stores, for example, conversation content and camera data of the day in the history data 2222 at the end of the day.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus and an action determination model at a predetermined timing, any of a plurality of types of device operations including non-operation as an action of the electronic apparatus; and
a storage control unit that stores, in history data, event data including an emotion value determined by the emotion determination unit and data including an action of the user, in which
the device operations include summarizing events of a previous day, and
in a case in which summarizing the events of the previous day is determined as the action of the electronic apparatus, the action determination unit adds a fixed sentence for giving an instruction for summarizing the events of the previous day to text representing the history data, inputs the text to the action determination model to acquire a summary of the events of the previous day, and outputs the acquired summary by speech or gesture in a case in which a conversation of the user who remembers the events of the previous day or a gesture of the user who thinks of something is detected when the action determination unit is activated at a predetermined time on the next day or when the user wakes up.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Tenth Embodiment

In the autonomous processing in the present embodiment, the action determination unit 2236 autonomously detects a state of the user 10. For example, the action determination unit 2236 autonomously detects a change in the body temperature of the user 10 at every predetermined timing. Specifically, the action determination unit 2236 detects a change in the body temperature of the user 10 by comparing the body temperature of the user 10 autonomously measured at every predetermined timing by the temperature sensor with the body temperature of the user 10 measured last time, the average body temperature of the user 10, or the like. As a temperature sensor, the temperature sensor included in the robot 100 may be applied, or a temperature sensor included in a device other than the robot 100 may be applied.

The action determination unit 2236 determines at least one of the emotion of the user 10 or the emotion of the robot 100 on the basis of the detected state of the user 10.

The action determination unit 2236 autonomously determines the surface temperature of the robot 100 according to at least one of the determined emotion of the user 10 or the determined emotion of the robot 100. For example, the action determination unit 2236 inputs text representing the determined emotion to the action determination model 2221. The action determination unit 2236 determines a surface temperature output by the action determination model 2221 as the surface temperature of the robot 100.

As a result, the user 10 can feel as if the robot 100 is alive. This is because, for example, even if there is no conversation between the user 10 and the robot 100 at various timings such as a case in which the user 10 is taking a nap together with the robot 100 or a case in which the user 10 is traveling, the surface temperature of the robot 100 autonomously changes according to at least one of the state of the user 10 or the state of the robot 100.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100, and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user. (4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The surface temperature of the robot is changed.

The action determination unit 2236 autonomously detects the state of the user 10 in a case in which it is determined that “(11) The surface temperature of the robot is changed.” as the robot action, and changes, in a case in which at least one of the emotion of the user 10 or the emotion of the robot 100 is determined on the basis of the detected state of the user 10, the surface temperature of the robot 100 according to at least one of the determined emotion of the user 10 or the determined emotion of the robot 100.

For example, the action determination unit 2236 changes the surface temperature of a part (for example, a hand or a face) of the robot 100 that the user 10 is likely to touch according to at least one of the determined emotion of the user 10 or the determined emotion of the robot 100. Specifically, in a case in which the robot 100 has an emotion of “happy”, the surface temperature of the hand of the robot 100 is more increased than before the robot 100 has an emotion of “happy”. In a case in which the robot 100 has an emotion of “angry”, the surface temperature of the face of the robot 100 is more increased than before the robot 100 has an emotion of “angry”.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include autonomously changing a surface temperature of the electronic apparatus, and
the action determination unit autonomously detects the state of the user as the action of the electronic apparatus, and determines, in a case in which at least one of the emotion of the user or the emotion of the electronic apparatus is determined on the basis of the detected state of the user, the surface temperature of the electronic apparatus according to at least one of the determined emotion of the user or the determined emotion of the electronic apparatus.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or the emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Eleventh Embodiment

In autonomous processing in the present embodiment, the action of the robot 100 includes determining an emotion of the robot 100 in consideration of events of the previous day. In a case in which it is determined to determine the emotion of the robot 100 in consideration of the events of the previous day as the action of the robot 100, the action determination unit 2232 adds a fixed sentence for giving an instruction for summarizing the events of the previous day to the text representing the history data 2222, and inputs the text to the action determination model 2221 to acquire a summary of the events of the previous day. The action determination unit 2232 adds a fixed sentence for inquiry about the emotion of the robot 100 on the next day to the text representing the history data 2222, and inputs the text to the action determination model 2221 to determine the emotion of the robot 100 in consideration of the acquired summary.

Specifically, the agent spontaneously and periodically detects a state of the user. For example, at the end of the day, the agent looks back all pieces of conversation content and camera data of the day, adds a fixed sentence of “Summarize this content” to text representing the history data 2222, and inputs the text to the sentence generation model to acquire a summary of the history of the previous day (for example, summarization is spontaneously performed by ChatGPT). A fixed sentence “What feeling should the agent have tomorrow?” is added to the text representing the history data 222 of the day, and the text is input to the sentence generation model to determine the emotion of the agent in consideration of the history of the previous day. The agent can also spontaneously take over the previous day's emotions on the next day and start a new day.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot determines the emotion of the robot in consideration of the events of the previous day.

The action determination unit 2236 inputs, to the sentence generation model, text representing the state of the user 10 and the state of the robot 100 recognized by the state recognition unit 2230, the current emotion value of the user 10 determined by the emotion determination unit 2232, and the current emotion value of the robot 100, and text for inquiry about any of a plurality of types of robot actions including no action every lapse of a certain period of time, and determines an action of the robot 100 on the basis of an output of the sentence generation model. Here, in a case in which there is no user 10 around the robot 100, the text to be input to the sentence generation model need not include the state of the user 10 and the current emotion value of the user 10, or may include the fact that there is no user 10. As an example, text such as “The robot is in a very pleasant state. The user is in a normally pleasant state. The user is sleeping. Which one of the following (1) to (11) is better as the action of the robot?
(1) The robot does nothing.
(2) The robot has a dream.
(3) The robot speaks to the user.
. . . ” is input to the sentence generation model. On the basis of the output “It can be said that either (1) the robot does nothing or (2) the robot has a dream is the most appropriate action.” of the sentence generation model, “(1) the robot does nothing” or “(2) the robot has a dream” is determined as the action of the robot 100.

As another example, text such as “The robot is in a slightly lonely state. The user is absent. The surroundings of the robot are dark. Which one of the following (1) to (11) is better as the action of the robot? (1) The robot does nothing.
(2) The robot has a dream.
(3) The robot speaks to the user.
. . . ” is input to the sentence generation model. On the basis of the output “It can be said that either (2) the robot has a dream or (4) the robot creates a picture diary is the most appropriate action.” of the sentence generation model, “(2) The robot has a dream” or “(4) The robot creates a picture diary.” is determined as the action of the robot 100.

In a case in which it is determined that, as the robot action, “(11) The robot determines the emotion of the robot in consideration of the events of the previous day.”, that is, the robot determines the emotion of the robot 100 in consideration of the events of the previous day, the action determination unit 2236 adds a fixed sentence for giving an instruction for summarizing the events of the previous day to the text representing the history data 2222, and inputs the text to the sentence generation model to acquire a summary of the events of the previous day. The action determination unit 2232 adds a fixed sentence for inquiry about the emotion of the robot 100 on the next day to the text representing the history data 2222 and inputs the text to the sentence generation model, thereby determining the emotion of the robot 100 in consideration of the acquired summary.

Regarding “(11) The robot determines the emotion of the robot in consideration of the events of the previous day.”, the storage control unit 2238 stores, for example, conversation content and camera data of the day in the history data 222 at the end of the day.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include determining the emotion of the electronic apparatus in consideration of events of a previous day, and
in a case in which it is determined to determine the emotion of the electronic apparatus in consideration of the events of the previous day as the action of the electronic apparatus, the action determination unit adds a fixed sentence for giving an instruction for summarizing the events of the previous day to text representing the history data, inputs the text to the action determination model to acquire a summary of the events of the previous day, adds a fixed sentence for inquiry about an emotion of the electronic apparatus on the next day to the text representing the history data, and inputs the text to the action determination model to determine the emotion of the electronic apparatus in consideration of the acquired summary.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twelfth Embodiment

In autonomous processing in the present embodiment, the action determination unit 2236 of the robot 100 spontaneously and periodically detects a state of the user. For example, at the end of the day, all pieces of conversation content and camera data of the day are looked back, and a fixed sentence “Summarize this content” is added to text representing the looked back content, and the text is input to the action determination model 2221 to acquire a summary of the history of the previous day of the user. That is, the summary of the actions of the user 10 on the previous day is spontaneously acquired by the action determination model 2221. In the morning of the next day, the action determination unit 2236 acquires the summary of the history of the previous day, inputs the acquired summary to a music generation engine, and acquires music summarizing the history of the previous day. The action control unit 2250 reproduces the acquired music. The music may be humming. In this case, for example, in a case in which the emotion of the user 10 on the previous day included in the history data 2222 is “happy”, music with a warm atmosphere is reproduced, and in a case in which the emotion is “angry”, music with an intense atmosphere is reproduced. Even if the user 10 is not having any conversation with the robot 100, the user 10 can feel as if the robot 100 is alive by spontaneously changing the music or humming performed by the robot 100 at all times on the basis of only the state of the user (states of conversation and emotion) and the state of the emotion of the robot.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100, and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot generates and reproduces music in consideration of events of the previous day.

In a case in which it is determined that “(11) The robot generates and reproduces music in consideration of events of the previous day.” as the robot action, the action determination unit 2236 selects the event data of the day from the history data 2222 at the end of the day, and looks back all the pieces of conversation content and event data of the day. The action determination unit 2236 adds a fixed sentence “Summarize this content” to text representing the looked-back content and inputs the text to the sentence generation model to acquire a summary of the history of the previous day. The summary reflects the actions and emotions of the user 10 on the previous day, and further the actions and emotions of the robot 100. The summary is stored in the storage unit 2220, for example. The action determination unit 2236 acquires the summary of the previous day in the morning of the next day, inputs the acquired summary to the music generation engine, and acquires music summarizing the history of the previous day. The action control unit 2250 reproduces the acquired music. A timing to reproduce the music is, for example, the time at which user 10 wakes up.

The music to be reproduced reflects the actions and emotions of the user 10 and the robot 100 on the previous day. For example, in a case in which the emotion of the user 10 based on the event data of the previous day included in the history data 2222 is “happy”, music with a warm atmosphere is reproduced, and in a case in which the emotion is “angry”, music with an intense atmosphere is reproduced. Music may be acquired and stored in the action schedule data 2224 while the user 10 is sleeping, and the music may be acquired from the action schedule data 224 and reproduced at the time at which the user 10 wakes up.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include generating and reproducing music in consideration of events of a previous day, and
in a case in which it is determined to generate and reproduce music in consideration of the events of the previous day as the action of the electronic apparatus, the action determination unit acquires a summary of event data of the previous day stored in the history data, generates music based on the summary, and reproduces the generated music.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or the emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Thirteenth Embodiment

In autonomous processing in the present embodiment, the action determination unit 2236 of the robot 100 spontaneously and periodically detects a state of the user. Specifically, one of action content of the robot 100 acquired by using a sentence generation model having an interaction function as the action determination model 2221 and action content determined by using the existing reaction rule as the action determination model 2221 is selected according to the intensity of the emotion of the robot 100 or the user 10. In a case in which the intensity of the emotion of the robot 100 or the user 10 is a threshold or more, the action determination unit 2236 selects action content determined by using the existing reaction rule. As a result, words spoken by and actions of the robot 100 become uniform, and even in a slightly different situation, in a case in which the emotion is a certain level or more, the robot 100 acts in the same manner, so that there is no deviation in action.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

In the case of determining an action of the robot 100, the action determination unit 2236 is configured to select one of action content to be taken by the robot 100 generated by using the sentence generation model as the action determination model 2221 and action content to be taken by the robot 100 determined on the basis of the reaction rule that is the action determination model 2221 according to the intensity of the emotion of the robot 100 or the user 10. In this case, the action determination unit 2236 compares the absolute value of the emotion value of the robot 100 or the user with a threshold, and selects the action content to be taken by the robot 100 determined by using the reaction rule in a case in which the absolute value is the threshold or more. In a case in which the emotion value is less than the threshold, the action determination unit 2236 selects the action content to be taken by the robot 100 generated by using the sentence generation model. For example, in a case in which the absolute value of the emotion value of the user is the threshold or more, or in a case in which the emotion value of the robot 100 is the threshold or more, the positive emotion or the negative emotion is strong, and thus the action determination unit 2236 determines the action content of the robot 100 by using the reaction rule. On the other hand, in a case in which the absolute value of the emotion value of the user is less than the threshold, or in a case in which the emotion value of the robot 100 is less than the threshold, the positive emotion or the negative emotion is weak, and thus the action determination unit 2236 generates an action to be taken by the robot 100 by using the sentence generation model.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines any of a plurality of types of device operations including non-operation as an action of the electronic apparatus by using at least one of the user state, the state of the electronic apparatus, the emotion of the user, or the emotion of the electronic apparatus, and an action determination model at a predetermined timing, in which the action determination unit selects, according to an intensity of the emotion of the user or the emotion of the electronic apparatus determined by the emotion determination unit, one of action content of the electronic apparatus generated by using a sentence generation model having an interaction function as the action determination model or action content determined by using, as the action determination model, a reaction rule for determining an action of the electronic apparatus according to the action of the user and the emotion of the user or the emotion of the electronic apparatus.

### (Supplementary Note 2)

The action control system according to Supplementary Note 1, in which the action determination unit selects the action content determined by using the reaction rule in a case in which an emotion value indicating the intensity of the emotion is a threshold or more, and selects the action content generated by using the sentence generation model in a case in which the emotion value is less than the threshold.

### (Supplementary Note 3)

The action control system according to Supplementary Note 1 or 2, in which the electronic apparatus is a robot, and
the action determination unit determines any of a plurality of types of robot actions including no action as an action of the robot.

### (Supplementary Note 4)

The action control system according to Supplementary Note 3, in which, in a case in which the action content is selected by using the sentence generation model, the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 5)

The action control system according to Supplementary Note 3, in which the robot is mounted on a stuffed toy or is connected to a control target device mounted on the stuffed toy in a wireless or wired manner.

### (Supplementary Note 6)

The action control system according to Supplementary Note 3, in which the robot is an agent for interacting with the user.

### Fourteenth Embodiment

In autonomous processing in the present embodiment, the action determination unit 2236 of the robot 100 spontaneously and periodically detects a state of the user. Specifically, the action determination unit 2236 calculates the degree of coincidence between a user action, a user emotion, and/or a robot emotion and a condition for an existing reaction rule that is the action determination model 2221, and selects action content determined by using the existing reaction rule in a case in which the degree of coincidence is high. In a case in which the degree of coincidence is low, action content determined by using a sentence generation model having an interaction function as the action determination model 2221 is selected. As a result, words spoken by and actions of the robot 100 become uniform, and even in a slightly different situation, the robot 100 acts in a similar manner, so that there is no deviation in action.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

Here, the reaction rule that is the action determination model 2221 defines an action of the robot 100 with respect to a condition for an action of the user, an emotion of the user, and/or an emotion of the robot 100. However, there is a case in which a condition for an action of the user, an emotion of the user, and/or an emotion of the robot 100 that has determined a certain action of the robot 100 does not completely match a condition for an actual action of the user, emotion of the user, and/or emotion of the robot 100 (hereinafter, referred to as a condition of the reaction rule). On the other hand, even in a case in which the condition of the reaction rule is not completely matched, it may be considered that the condition of the reaction rule is matched.

In the present embodiment, in the case of determining the action of the robot 100, the action determination unit 2236 calculates the degree of coincidence between the action of the user, the emotion of the user, and/or the emotion of the robot 100 and the condition of the reaction rule that is the action determination model 2221. In a case in which the degree of coincidence is high, that is, in a case in which the degree of coincidence is a threshold or more, the action determination unit 2236 selects the action content determined by using the reaction rule. On the other hand, in a case in which the degree of coincidence is low, that is, in a case in which the degree of coincidence is less than the threshold, the action determination unit 2236 selects the action content determined by using the sentence generation model. Here, the degree of coincidence being the threshold or more indicates that the condition of the reaction rule is not completely matched, but the condition is matched to such an extent that the condition can be regarded as being matched.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the action determination unit calculates a degree of coincidence between the action of the user, the emotion of the user, and/or the emotion of the electronic apparatus and a condition of a reaction rule for determining the action of the electronic apparatus according to the action of the user, the emotion of the user, and/or the emotion of the electronic apparatus, selects action content determined by using the reaction rule in a case in which the degree of coincidence is a threshold or more, and selects action content of the electronic apparatus determined by using a sentence generation model having an interaction function as the action determination model in a case in which the degree of coincidence is less than the threshold.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which, in a case in which the action content is determined by using the sentence generation model, the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Fifteenth Embodiment

In autonomous processing in the present embodiment, the robot 100 spontaneously and periodically detects a state of the user 10. Specifically, the robot 100 spontaneously and periodically detects the action of the user 10, the emotion of the user 10, and the emotion of the robot 100, adds a fixed sentence for inquiry about a gesture to be taken by the robot 100 to text representing the state of the user 10, inputs the text to the sentence generation model, and acquires a gesture of the robot 100. The gesture is acquired and stored, and the stored gesture is activated at another timing, for example. As a result, the robot 100 can spontaneously detect the state of the user 10, determine a gesture of the robot 100 in advance, and in a case in which there is a certain trigger for the user 10 next time, the robot 100 itself can execute the gesture.

The robot 100 spontaneously and periodically detects the action of the user 10, the emotion of the user 10, and the emotion of the robot 100, adds a fixed sentence for inquiry about the speech content to be taken by the robot 100 to text representing the state of the user 10, and inputs the text to the sentence generation model to acquire the speech content of the robot 100. This speech content is acquired and stored, and the stored speech content is activated at another timing, for example. As a result, the robot 100 spontaneously detects the state of the user 10, determines the speech content of the robot 100 in advance, and in a case in which there is a certain trigger for the user 10 next time, the robot 100 itself says the speech content. The robot 100 may perform only a gesture, may perform only a speech, or may perform a speech together with a gesture in a case in which there is a certain trigger.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) An action schedule of the robot is determined in advance.

In a case in which it is determined that “(11) An action schedule of the robot is determined.”, for example, a gesture of the robot 100 is determined in advance as the robot action, the action determination unit 2236 determines an activation condition for activating a gesture and stores the determined activation condition in the action schedule data 2224. In a case in which there are a plurality of gestures, activation conditions for activating the respective gestures are determined and stored in the action schedule data 2224.

Specifically, text representing the state of the user 10 and the state of the robot 100 recognized by the state recognition unit 2230, the current emotion value of the user 10 determined by the emotion determination unit 2232, the current emotion value of the robot 100, and the history data 2222, and text for inquiry about the robot actions (gestures) to be executed later and the activation conditions are input to the sentence generation model, and an activation condition for activating a gesture is determined on the basis of an output of the sentence generation model. Here, the activation condition is, for example, detection of the user 10.

In a case in which the activation condition for the action schedule data 2224 is satisfied, the action determination unit 2236 determines, as the action of the robot 100, execution of a gesture that satisfies the activation condition.

For example, in a case in which it is determined to determine in advance the speech content of the robot 100 as the robot action, the action determination unit 2236 determines an activation condition for saying the speech content and stores the determined activation condition in the action schedule data 2224. In a case in which there are a plurality of pieces of speech content, an activation condition for saying each piece of speech content is determined and stored in the action schedule data 2224.

Specifically, text representing the state of the user 10 and the state of the robot 100 recognized by the state recognition unit 2230, the current emotion value of the user 10 determined by the emotion determination unit 2232, the current emotion value of the robot 100, and the history data 2222, and text for inquiry about the robot actions (speech) to be executed later and the activation conditions are input to the sentence generation model, and an activation condition for saying speech content is determined on the basis of an output of the sentence generation model. Here, the activation condition is, for example, detection of the user 10.

In a case in which the activation condition for the action schedule data 2224 is satisfied, the action determination unit 2236 determines, as the action of the robot 100, to say speech content that satisfies the activation condition.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include determining in advance a gesture of the electronic apparatus, and
in a case in which it is determined to determine in advance a gesture of the electronic apparatus as the action of the electronic apparatus, the action determination unit determines an activation condition for activating the gesture and stores the activation condition in action schedule data, and
determines to execute the gesture in a case in which the activation condition for the action schedule data is satisfied.

### (Supplementary Note 2)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include determining in advance speech content of the electronic apparatus, and
in a case in which it is determined to determine in advance speech content of the electronic apparatus as the action of the electronic apparatus, the action determination unit determines an activation condition for saying the speech content and stores the activation condition in action schedule data, and
determines to say the speech content in a case in which the activation condition for the action schedule data is satisfied.

### (Supplementary Note 3)

The action control system according to Supplementary Note 1 or 2, in which the electronic apparatus is a robot, and the action determination unit determines any of a plurality of types of robot actions including no action as an action of the robot.

### (Supplementary Note 4)

The action control system according to Supplementary Note 3, in which the action determination model is a sentence generation model having an interaction function, and the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 5)

The action control system according to Supplementary Note 3, in which the robot is mounted on a stuffed toy or is connected to a control target device mounted on the stuffed toy in a wireless or wired manner.

### (Supplementary Note 6)

The action control system according to Supplementary Note 3, in which the robot is an agent for interacting with the user.

### Sixteenth Embodiment

In autonomous processing in the present embodiment, the action determination unit 2236 spontaneously and periodically detects a state of the user 10. The action determination unit 2236 spontaneously estimates a culture area (also referred to as a language area) in which the user 10 resides, and reflects the estimated culture area in generation of an answer by a sentence generation model using AI as an example of the action determination model 2221, determination of the emotion of the user 10 by the emotion determination unit 2232, and determination of the emotion of the robot 100 by the emotion determination unit 2232. For example, in a case in which it is estimated that the user 10 resides in the Kansai area or in a case in which it is detected that the user 10 is speaking the Kansai dialect, the robot 100 spontaneously switches to the brain of the Kansai area. In this case, the robot 100 makes a gesture of putting a thrust or makes a speech such as “Why?”. The action determination unit 2236 may reflect the estimated culture area in one or two of the generation of an answer by the sentence generation model, the determination of the emotion of the user 10 by the emotion determination unit 2232, and the determination of the emotion of the robot 100 by the emotion determination unit 2232.

The action determination unit 2236 may estimate a culture area of the user 10 by using various methods. For example, the action determination unit 2236 may estimate the culture area of the user 10 from a conversation of the user 10. The “conversation of the user 10” here may be interpreted to include a conversation between the user 10 and another robot, a conversation between the users 10, and a soliloquy of the user 10, in addition to the interaction between the user 10 and the robot 100. That is, the action determination unit 2236 may infer the culture area of the user 10 from the conversation of the user 10 that the robot 100 itself has attended without being a party, in addition to the interaction with the user 10 in which the robot 100 itself is a party. As an example, in a case in which the user 10 frequently talks about Osaka prefecture in a conversation, or in a case in which local information of Osaka prefecture is said as a topic, the action determination unit 2236 may estimate that the culture area of the user 10 is the Kansai area. The action determination unit 2236 may estimate that the culture area of the user 10 is the Kansai area in a case in which the user uses the Kansai dialect in the conversation. Alternatively or in addition, the action determination unit 2236 may estimate the culture area of the user 10 on the basis of position information. As an example, the action determination unit 2236 may store in advance a culture area map in which position information and a culture area are associated with each other, and in a case in which position information measured by positioning means such as a global positioning system (GPS) is associated with the Kansai area, the action determination unit may estimate that the culture area of the user 10 is the Kansai area. In this case, when viewed from the user 10, the robot 100 is transformed into a character in the Kansai area without being ascertained by the user 10.

As described above, the robot 100 acts in accordance with the residential culture of the user 10, whereby the user experience can be improved.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

In a case in which it is determined that the robot 100 speaks in the actions (1) to (10) described above as the robot action, the action determination unit 2236 uses the sentence generation model to determine speech content according to the estimated culture area of the user 10 as the speech content of the robot 100 corresponding to the user state and the emotion of the user 10 or the emotion of the robot 100. For example, in a case in which the culture area of the user 10 estimated by the action determination unit 2236 is in the Kansai area, the voice representing the speech content output by the robot 100 is a voice in the Kansai dialect, such as a speech “Why?”. The action determination unit 2236 may make a gesture corresponding to the speech content as a gesture corresponding to the estimated culture area of the user 10, such as a gesture of putting a thrust.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the action determination unit reflects an estimated culture area of the user in at least one of output generation by the action determination model, determination of the emotion of the user by the emotion determination unit, or determination of the emotion of the electronic apparatus by the emotion determination unit.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Seventeenth Embodiment

In autonomous processing in the present embodiment, the robot 100 as an agent spontaneously and periodically detects a state of the user. More specifically, the robot 100 spontaneously and periodically detects whether the user and his/her family use a social networking service (hereinafter, referred to as an SNS). That is, the robot 100 constantly monitors a display of a smartphone or the like owned by each of the user and his/her family, and detects a use state of the SNS. In a case in which the user is a child, the robot 100 spontaneously considers a way of facing the SNS and posted contents while talking with the child.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot gives advice regarding the SNS to the user.

In a case in which the action determination unit 2236 determines that that “(11) The robot gives advice regarding the SNS to the user.”, that is, the advice regarding the SNS will be given to the user, as the robot action, the robot 100 determines speech content of the robot corresponding to the information stored in the collected data 2223 by using the sentence generation model. In this case, the action control unit 2250 causes a speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

Specifically, the robot 100 proposes a way of facing the SNS for the user to appropriately use the SNS such as reassurance and safety, and proposes post content on the SNS while conversing with the user. For example, the robot 100 proposes a combination of one or more of information security measures, protection of personal information, prohibition of defamation, prohibition of spread of false information, and compliance with laws to the user as a way of facing the SNS. As a specific example, the robot 100 can propose a way of facing the SNS such as “It is better to be careful not to disclose personal information on the Internet!” in response to the question “What should be noted when using the SNS?” in the conversation with the user.

On the other hand, the robot 100 proposes, to the user, post content that satisfies a predetermined condition including a combination of one or more of information security measures, protection of personal information, prohibition of defamation, prohibition of spread of false information, and compliance with laws. As a specific example, in response to a speech such as “I want to post something about A and B that won't cause an uproar,” in the conversation with the user, the robot 100 can think of post content that does not slander either party, such as “A and B are both great!”, and propose the post content to the user.

In a case in which the robot 100 recognizes the user as a minor, the robot makes a proposal while talking to the user about one or both of the way of facing the SNS corresponding to the minor and the posted content on the SNS. Specifically, the robot 100 can make a proposal for a way of facing the SNS and the content posted on the SNS on the basis of a stricter condition corresponding to a minor. As a specific example, the robot 100 can propose a way of facing the SNS such as “Please be careful not to spread disclosure of personal information, slander, or false information!” in response to the question “What should be noted when using the SNS?” in the conversation with the minor user. In response to a speech such as “I want to post something about A and B that won't cause an uproar,” in the conversation with the minor user, the robot 100 can think of a post that is polite and does not slander either party, such as “A and B are both great!”, and propose the post to the user.

The robot 100 can perform a speech related to the posting when the user finishes the posting to the SNS regarding the post content on the SNS as the action of making the proposal related to the use of the SNS. For example, the robot 100 can spontaneously perform a speech with content such as “In this post, you are conscious of a way of facing the SNS, and the score is 100 points!” after the user finishes posting to the SNS.

The robot 100 can analyze the post content posted by the user, and make a proposal on a way of facing the SNS or how to create post content to the user on the basis of the analysis result. For example, in a case in which there is no speech from the user, the robot 100 can perform a speech with content such as “This post content contains content different from the fact and may be false information, so be careful!” on the basis of the post content of the user.

The robot 100 proposes, to the user, one or both of a way of facing the SNS or the post content on the SNS in a conversation form on the basis of a state or an action of the user. For example, in a case in which the user holds a terminal device in his/her hand and recognizes that “The user may be in trouble with how to use the SNS.”, the robot 100 can talk to the user in a conversation form and propose a method of using the SNS, a way of facing the SNS, and post content.

Regarding “(11) The robot gives advice regarding the SNS to the user.”, the related information collection unit 2270 acquires information regarding the SNS. For example, the related information collection unit 2270 may periodically access an information source such as a television or a web by itself, spontaneously collect information regarding laws and regulations, incidents, problems, or the like related to the SNS, and store the collected information in the collected data 2233. As a result, since the robot 100 can acquire the latest information regarding the SNS, it is possible to spontaneously give advice corresponding to the latest problem or the like regarding the SNS to the user.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include giving the user advice regarding a social networking service, and
the action determination unit gives the user the advice regarding the social networking service in a case in which it is determined to give the user the advice regarding the social networking service as the action of the electronic apparatus.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Eighteenth Embodiment

In autonomous processing in the present embodiment, the robot 100 (agent) has a heart (acts as if having a heart), autonomously (spontaneously), and periodically checks a health condition of the user 10. More specifically, the action determination unit 2236 detects a parameter representing the health condition of the user 10 autonomously and periodically via the sensor unit 2200. Examples of the parameter representing the health condition of the user 10 include intonation of a conversation of the user 10, a complexion of the user 10, shaking of a hand of the user 10, a body temperature of the user 10 measured by a thermo sensor, a respiratory rate of the user 10, a heart rate of the user 10, a sleeping time of the user 10, and the number of times of entry of the user 10 into a toilet. In a case in which the user 10 wears a wearable device having a function of measuring blood pressure, blood glucose level, and the like, it is also possible to acquire the blood pressure, blood glucose level, and the like of the user 10 by performing wireless communication with the wearable device. The detected parameter representing the health condition of the user 10 is stored in time series as history data 2222 by the storage control unit 2238.

The action determination unit 2236 checks the health condition of the user 10 by using the action determination model 2221 on the basis of the parameter representing the health condition of the user 10 stored in time series as the history data 2222 (determines whether or not to talk to the user 10 or to recommend drug to the user 10). The action determination unit 2236 autonomously talks to the user 10 to watch over the user 10 as necessary, autonomously concerns the health of the user 10, autonomously determines the symptom of the user 10 without being heard by the user, and recommends the user 10 to take an appropriate medicine as necessary.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot gives health advice to the user.

In a case in which it is determined that, as the robot action, “(11) The robot gives health advice to the user.”, that is, that the robot 100 makes a speech regarding the health of the user 10, the health condition of the user 10 is checked by inputting, to the sentence generation model, a parameter representing the health condition of the user 10 stored in time series as the history data 2222, and speech content of the robot regarding the health condition of the user 10 is determined. In this case, the action control unit 2250 causes a speaker included in the control target 2252 to output a voice representing the determined speech content of the robot. in a case in which the user 10 is absent around the robot 100, the action control unit 2250 stores the determined speech content of the robot in the action schedule data 2224 without outputting a voice representing the determined speech content of the robot.

As an example, the action determination unit 2236 inputs, to the sentence generation model, text such as “Regarding the parameter representing the health condition of the user, the body temperature of the user changes to T1 (t1), T2 (t2), and T3 (t3). Which of the following (11a) to (11c) is better as the action of the robot?
(11a) The robot does nothing.
(11b) The robot talks to the user about words that concern the physical condition.
(11c) The robot recommends the user to take medicine.”

Here, in a case in which the output of the sentence generation model is “It can be said that (11b) The robot talks to the user about words that concern the physical condition and (11c) The robot recommends the user to take medicine are appropriate actions.”, the action determination unit 2236 determines that “(11b) The robot talks to the user about words that concern the physical condition” and “(11c) The robot recommends the user to take medicine” as the actions of the robot 100 on the basis of the output. In a case in which the output of the sentence generation model includes “(11c) The robot recommends the user to take medicine” as described above, the action determination unit 2236 further inputs text such as “What medicine do you recommend the user to take?” to the sentence generation model. Here, in a case in which the output of the sentence generation model is “The medicine recommended to the user is X.”, the action determination unit 236 determines a speech of “I recommend to take medicine X” as the action of the robot 100 on the basis of the output.

Regarding “(11) The robot gives health advice to the user.”, the storage control unit 2238 stores a parameter representing the health condition of the user 10 detected autonomously and periodically as the chronological history data 2222.

In the above example, the aspect of determining to give the health advice to the user in a case in which the output of the sentence generation model is the content of recommending “(11) The robot gives health advice to the user.” has been described. However, the disclosed technology is not limited thereto, and the action determination unit 2236 may autonomously check the health condition of the user 10 on the basis of the parameter representing the health condition of the user 10, and may determine to give health advice to the user in a case in which it is determined that there is some abnormality in the health condition of the user 10. The health condition of the user 10 may be autonomously checked, for example, by comparing a detected parameter representing the health condition of the user 10 with a preset threshold, or by inputting the detected parameter representing the health condition of the user 10 to a neural network trained in advance and acquiring an evaluation value for evaluating the health condition of the user 10.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include giving health advice to the user, and
the storage control unit stores, in the history data, a parameter representing a detected health condition of the user, and
in a case in which it is determined to give health advice to the user as an action of the electronic apparatus, the action determination unit autonomously determines an action corresponding to the health condition of the user on the basis of the parameter representing the health condition of the user.

### (Supplementary Note 2)

The action control system according to Supplementary Note 1, in which the parameter representing the health condition of the user is at least one of intonation of a conversation of the user, a complexion of the user, shaking of a hand of the user, a body temperature of the user, a respiratory rate of the user, a sleeping time of the user, the number of times the user has entered a toilet, a heart rate of the user, a blood pressure of the user, or a blood glucose level of the user.

### (Supplementary Note 3)

The action control system according to Supplementary Note 1, in which the electronic apparatus is a robot, and
the action determination unit determines any of a plurality of types of robot actions including no action as an action of the robot.

### (Supplementary Note 4)

The action control system according to Supplementary Note 3, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 5)

The action control system according to Supplementary Note 3 or 4, in which the robot is mounted on a stuffed toy or is connected to a control target device mounted on the stuffed toy in a wireless or wired manner.

### (Supplementary Note 6)

The action control system according to Supplementary Note 3 or 4, in which the robot is an agent for interacting with the user.

### Nineteenth Embodiment

In autonomous processing in the present embodiment, the robot 100 spontaneously and periodically detects a state of the user 10. For example, the robot 100 constantly detects a hobby, a preference, or the like of the user 10, and proposes to go to an art gallery, a museum, or the like according to a holiday of the user 10 in a case in which the hobby of the user 10 relates to an art gallery, a museum, an exhibition, or the like. In a case in which the user 10 goes to an art gallery or a museum, the robot 100 functions as an agent that selects an exhibit matching the hobby or the preference of the user 10, and has a conversation while having fun together by explaining the exhibit.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (12).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot proposes art galleries, museums, and exhibitions to which the user should go.
(12) The robot introduces an event that the user should participate in.

In a case in which it is determined that “(11) The robot proposes art galleries, museums, and exhibitions to which the user should go.”, that is, an action of the user 10 is proposed as the robot action, the action determination unit 2236 determines a proposed destination by using the sentence generation model on the basis of the event data stored in the history data 2222. In this case, the action determination unit 2236 makes a proposal in accordance with a schedule or a plan of the user 10 acquired in advance. The action control unit 2250 causes a speaker included in the control target 2252 to output a voice for proposing an action of the user 10. In a case in which the user 10 is absent around the robot 100, the action control unit 2250 stores proposal of the action of the user in the action schedule data 224 without outputting a voice for proposing the action of the user.

Regarding “(11) The robot proposes art galleries, museums, and exhibitions to which the user should go.”, the related information collection unit 2270 acquires information regarding art galleries, museums, and exhibitions of interest of the user 10. For example, the related information collection unit 2270 periodically collects information regarding art galleries, museums, and exhibitions present within a predetermined range from the current location of the user from external data by using ChatGPT Plugins.

In a case in which it is determined that “(12) The robot introduces an event that the user should participate in.” as the robot action, the action determination unit 2236 determines speech content of the robot corresponding to the information stored in the collected data 2223 by using the sentence generation model. In this case, the action control unit 2250 causes a speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot. In this case, the action determination unit 2236 introduces an event that allows the user 10 to participate in a free time period in accordance with the schedule or plan of the user 10 acquired in advance.

Regarding “(12) The robot introduces an event that the user should participate in.”, the related information collection unit 2270 acquires information regarding an event of interest of the user 10. For example, the related information collection unit 2270 periodically collects information regarding an event scheduled to be held within a predetermined range from the current location of the user from external data by using ChatGPT Plugins.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include proposing to go to an art gallery, a museum, and an exhibition in accordance with a schedule of the user, and
in a case in which it is determined to propose to go to an art gallery, a museum, or an exhibition as the action of the electronic apparatus, the action determination unit determines a proposed destination by using a sentence generation model on the basis of the event data stored in the history data.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twentieth Embodiment

In autonomous processing in the present embodiment, the agent spontaneously and periodically detects a state of the user. The agent constantly detects a hobby/preference of the user, stores characteristics of the user, and ascertains a music hobby that is the user's hobby. The agent spontaneously reproduces a favorite song according to the user's situation.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot reproduces the user's favorite music.

In a case in which it is determined that “(11) The robot reproduces the user's favorite music”, that is, the robot reproduces music suitable for the user 10 as the robot action, the action determination unit 2236 determines music to be reproduced on the basis of the information stored in the collected data 2223. Alternatively, the action determination unit 2236 may determine music to be reproduced on the basis of the event data stored in the history data 2222. In this case, the action control unit 2250 causes a speaker included in the control target 2252 to output the music. in a case in which the user 10 is absent around the robot 100, the action control unit 2250 stores, in the action schedule data 2224, reproducing music preferred by the user 10 without outputting music.

Regarding “(11) The robot reproduces the user's favorite music”, the related information collection unit 2270 stores necessary information related to the user's taste in music in the collected data 2223. The necessary information related to the user's taste in music includes at least one of a taste in music type, a taste in musical instrument, or a taste in singer.

Examples of the music type include genres such as jazz, classical, rock, and pop. In a case in which the action determination unit 2236 determines music to be reproduced on the basis of the user's taste in music type, the action determination unit 2236 determines music included in a genre of music that the user likes as music to be reproduced.

Examples of the musical instrument include various musical instruments such as wind instruments, string instruments, and percussive instruments. In a case in which the action determination unit 2236 determines music to be reproduced on the basis of the user's taste in the musical instrument, the action determination unit 2236 determines music in which the user's favorite musical instrument is used as music to be reproduced.

Examples of the singer include not only a specific artist name but also no singer. In a case in which the action determination unit 2236 determines music to be reproduced on the basis of the user's taste in a singer, the action determination unit 2236 determines music sung by the user's favorite singer as music to be reproduced. Alternatively, music without a singer (so-called instrumental music) is determined as music to be reproduced.

The necessary information related to the user's taste in music may include a taste in the volume level of music to be output from the speaker.

Regarding “(11) The robot reproduces the user's favorite music”, the storage control unit 2238 stores necessary data in the history data 2222.

The robot 100 may be applied to a music reproduction device such as an AI speaker or an acoustic device (audio device) such as a radio.

The robot 100 as an acoustic device includes a storage unit that stores music data, a conversion unit such as a D/A converter that converts the music data into sound, and a speaker that outputs the sound. In a case in which the robot 100 is mounted on a radio, the robot 100 includes a tuner unit that receives radio waves of radio broadcasting and outputs sound.

The action determination unit 2236 can determine music to be reproduced in accordance with the user's taste, the user's situation, and the user's reaction.

In this case, the action determination unit 2236 can determine to reproduce music suitable for the emotion of the user 10 at that time by considering not only the taste regarding the music of the user 10 but also the emotion of the user 10 and the history data 2222. By considering the emotion of the robot 100, it is possible to make the user 10 feel that the robot 100 has an emotion. For example, even if the taste of the user 10 in music is classical music, in a case in which it is determined that it is better to encourage the user 10, the robot 100 can perform control to select and reproduce lightweight popular music with a fast tempo.

The robot 100 reproduces music and acquires a reaction of the user 10. Specifically, the action control unit 2250 reproduces the music determined by the action determination unit 2236. The state recognition unit 2230 recognizes a state of the user 10 on the basis of the information analyzed by the sensor module unit 2210. The emotion determination unit 2232 determines an emotion value indicating the emotion of the user 10 on the basis of the information analyzed by the sensor module unit 2210 and the state of the user 10 recognized by the state recognition unit 2230.

The action determination unit 2236 determines whether or not the reaction of the user 10 is positive on the basis of the state of the user 10 recognized by the state recognition unit 2230 and the emotion value indicating the emotion of the user 10. The action determination unit 2236 determines, as the action of the robot 100, whether to continue reproducing the same music, reproduce another music of the same genre as the reproduced music, reproduce music of a different genre from the reproduced music, or stop reproducing the music.

For example, in a case in which the reaction of the user 10 is positive, the robot 100 continues reproducing the same music. Alternatively, after the end of the music being reproduced, another music of the same genre as the music is reproduced.

Specifically, in a case in which the action determination unit 2236 determines to continue reproducing the same music as the action of the robot 100, the action control unit 2250 controls an acoustic device that is the control target 2252 to repeatedly continue reproducing the same music. Alternatively, in a case in which it is determined that another music of the same genre as the reproduced music is to be reproduced as the action of the robot 100, the action control unit 2250 controls the acoustic device that is the control target 2252 to reproduce different music of the same genre as the music after the end of the music being reproduced.

In a case in which the reaction of the user 10 is not positive, the robot 100 reproduces music of a genre different from the reproduced music. Alternatively, the reproduction of the music is stopped.

Specifically, in a case in which the action determination unit 2236 determines to reproduce music of a genre different from the reproduced music as the action of the robot 100, the action control unit 2250 controls the acoustic device that is the control target 2252 to reproduce music of a genre different from the reproduced music. Alternatively, in a case in which the action determination unit 2236 determines to stop reproducing the music as the action of the robot 100, the action control unit 2250 controls the acoustic device that is the control target 2252 to reproduce music of a genre different from the reproduced music.

As described above, the robot 100 can execute processing of selecting a genre of music to be reproduced in accordance with the user's taste, the user's situation, and the user's reaction, and reproducing music included in the selected genre.

In the above description, a case in which the action determination unit 2236 determines music to be output from the acoustic device has been described, but a volume level for reproducing music may be determined.

For example, in a case in which a taste in the volume level of music to be output from the speaker is stored in the collected data 2223, the action determination unit 2236 determines the volume level of music to be reproduced according to the user's taste in the volume level. In a case in which it is determined that the tension of the emotion of the user 10 is not too high, the action determination unit 2236 may perform control to lower the volume level of the music to be reproduced.

In a case in which the acoustic device mounted on the robot 100 is a radio, the action determination unit 2236 can select a broadcast station to be received and perform control such that the selected broadcast station is tuned. For example, in a case in which it is determined that the tension of the emotion of the user 10 is not too high, the action determination unit 2236 can perform control to tune a broadcast station mainly broadcasting classical music. On the other hand, in a case in which the tension of the emotion of the user 10 is determined to be relatively high, the action determination unit 2236 can perform control to tune a broadcast station mainly broadcasting rock music.

In a case in which the robot 100 acquires and stores information such as a broadcast program guide broadcast by a broadcast station, it is possible to specify a broadcast program being broadcast by each broadcast station from the current time and information such as the broadcast program guide. Therefore, in a case in which the information such as the broadcast program guide broadcast by the broadcast station is stored, the action determination unit 2236 may tune the broadcast station by using the information.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include reproducing user's favorite music, and
in a case in which it is determined to reproduce user's favorite music as the action of the electronic apparatus, the action determination unit determines music to be reproduced on the basis of information regarding the user's taste in music stored in a storage unit.

### (Supplementary Note 2)

The action control system according to Supplementary Note 1, in which the action determination unit determines the music to be reproduced on the basis of at least one of a taste in a type of music, a taste in a musical instrument, or a taste in a singer as the information regarding the user's taste in music.

### (Supplementary Note 3)

The action control system according to Supplementary Note 1 or 2, in which the action determination unit determines a volume level according to the user's taste in a volume level.

### (Supplementary Note 4)

The action control system according to Supplementary Note 1, in which the electronic apparatus is a robot, and
the action determination unit determines any of a plurality of types of robot actions including no action as an action of the robot.

### (Supplementary Note 5)

The action control system according to Supplementary Note 4, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 6)

The action control system according to Supplementary Note 4 or 5, in which the robot is mounted on a stuffed toy or is connected to a control target device mounted on the stuffed toy in a wireless or wired manner.

### (Supplementary Note 7)

The action control system according to Supplementary Note 4 or 5, in which the robot is an agent for interacting with the user.

### Twenty-First Embodiment

In autonomous processing in the present embodiment, the robot 100 spontaneously and periodically detects a state of the user 10. The robot 100 constantly detects a hobby/preference of the user 10, and the robot 100 stores the detected hobby/preference of the user 10 as a characteristic of the user 10, and ascertains in advance what kind of web (web site) the hobby/preference of the user 10 is interested in. The robot 100 has a mind and spontaneously proposes to the user 10 what kind of website seems to be fun. The robot 100 enjoys the website together with the user 10, and finds information for enjoying by itself.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model. For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot proposes a website recommended to the user.

In a case in which it is determined, as the robot action, that “(11) The robot proposes a website recommended to the user.”, that is, a recommended website is proposed to the user 10, the action determination unit 2236 ascertains a website that the user 10 can enjoy on the basis of the hobby/preference stored as the characteristic of the user 10, and determines a website to be proposed to the user 10. In this case, the action control unit 2250 causes the display device included in the control target 2252 to display the website determined as the favorite information of the user 10. In a case in which the user 10 is absent around the robot 100, the action control unit 2250 stores the determined website in the action schedule data 2224 without displaying the determined website on the display device.

Regarding “(11) The robot proposes a website recommended to the user.”, the related information collection unit 2270 collects information regarding web sites related to the hobby/preference of the user 10 from external data (web sites such as news sites and moving image sites) as taste information of the user 10 on the basis of the characteristic information of the user 10 according to the hobby/preference acquired for the user 10 at a predetermined timing.

Regarding “(11) The robot proposes a website recommended to the user.”, the storage control unit 2238 stores the collected information regarding the websites in the history data 2222, and stores proposal of the information regarding the websites to the user 10 in the action schedule data 2224.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include acquiring, as the event data, external data based on taste information of the user as the emotion of the user and outputting an image or a sound according to the event data, and
in a case in which it is determined to output the event data based on the taste information of the user, the action determination unit outputs the determined event data through the device operations.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Second Embodiment

In autonomous processing in the present embodiment, the agent spontaneously and periodically detects a state of the user. The agent constantly detects a hobby/preference of the user, stores characteristics of the user, and ascertains what kind of shopping the user likes. The agent spontaneously proposes to the user to go shopping, and enjoys shopping while having a conversation with the user.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

In a case in which it is determined that “(5) The robot proposes an activity.”, that is, an activity is proposed to the user, as the robot action, the action determination unit 2236 spontaneously proposes an activity to the user.

Regarding “(5) The robot proposes an activity.”, the state recognition unit 2230 spontaneously and periodically detects a state of the user, so that the agent constantly detects the hobby/preference of the user. The agent stores the characteristics of the user, and ascertains what kind of shopping the user likes, for example.

The action determination unit 2236 spontaneously proposes to the user to go shopping, for example, as the robot action. As a result, the agent enjoys shopping while having a conversation with the user.

Regarding “(5) The robot proposes an activity.”, the related information collection unit 2270 spontaneously collects information related to the taste information from the external data (web sites such as news sites and moving image sites) on the basis of the taste information acquired for the user 10.

Regarding “(5) The robot proposes an activity.”, the storage control unit 2238 stores, for example, an activity proposed to the user in the history data 2222.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus, in which
the device operations include spontaneously and periodically detecting a state of the user, and
the action determination unit spontaneously proposes an activity to the user in a case in which it is determined to propose the activity to the user as the action of the electronic apparatus.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Third Embodiment

In autonomous processing in the present embodiment, the robot 100 as an agent spontaneously and periodically detects a state of the user 10. The robot 100 constantly detects a hobby/preference of the user, stores characteristics of the user 10, and ascertains in advance what kind of eating and drinking the user 10 likes. The robot 100 proposes an activity related to eating and drinking according to the emotion value of the user 10 and/or the robot 100. For example, the robot 100 may propose the user 10 and/or a person around the user 10 to go to an eating place at a certain timing according to the emotion value of the user 10 and/or the robot 100. The robot 100 may spontaneously propose a menu to the user 10 or spontaneously order a menu from a store clerk of the eating place on the basis of the hobby/preference of the user 10 in the restaurant according to the emotion value of the user 10 and/or the robot 100.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (10).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.

In a case in which it is determined that “(5) The robot proposes an activity.”, that is, an action of the user 10 is proposed, as the robot action, the action determination unit 2236 determines an action of the user to be spontaneously proposed by using the sentence generation model on the basis of the event data stored in the history data 2222. In this case, the action control unit 2250 causes a speaker included in the control target 2252 to output a voice for proposing an action of the user. In a case in which the user 10 is absent around the robot 100, the action control unit 2250 stores proposal of the action of the user in the action schedule data 2224 without outputting a voice for proposing the action of the user.

For example, in a case in which it is determined to propose an activity related to eating and drinking as the activity, an action related to eating and drinking of the user to be spontaneously proposed is determined by using the sentence generation model on the basis of the event data stored in the history data 2222. Specifically, the user may be prompted to go to an eating place, or a menu for eating and drinking in an eating place may be proposed.

In a case in which it is determined that, as the robot action, “(6) The robot proposes a person with whom the user should meet.”, that is, the robot proposes a partner who should have a contact with the user 10, the action determination unit 2236 determines a proposed partner who should have a contact with the user by using the sentence generation model on the basis of the event data stored in the history data 2222. In this case, the action control unit 2250 causes a speaker included in the control target 252 to output a voice representing that a partner who should have a contact with the user is proposed. In a case in which the user 10 is absent around the robot 100, the action control unit 2250 stores proposal of a partner who should have a contact with the user in the action schedule data 2224 without outputting a voice representing proposal of a partner who should have a contact with the user.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include proposing an activity related to eating and drinking, and
in a case in which it is determined to propose an activity related to eating and drinking as the action of the electronic apparatus, the action determination unit proposes the activity related to eating and drinking.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Fourth Embodiment

In autonomous processing in the present embodiment, the robot 100 spontaneously and periodically detects a state of the user 10. The robot 100 constantly detects a hobby/preference of the user 10, stores the characteristics of the user 10, and spontaneously predicts the future schedule of the user 10 from the conversation of the user 10. The robot 100 has a mind, and spontaneously makes a schedule according to the taste, situation, and reaction of the user 10. In a case in which there is a schedule that the user does not want to go, the robot 100 makes a notification of rejection.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot determines a schedule of the user.

In a case in which it is determined that “(11) The robot determines a schedule of the user.”, that is, a schedule is proposed, as the robot action, the action determination unit 2236 determines a schedule of the user to be proposed by using the sentence generation model on the basis of the event data stored in the history data 2222. In this case, the action control unit 2250 causes a speaker included in the control target 252 to output a voice for proposing an action of the user.

Regarding “(11) The robot determines a schedule of the user.”, the related information collection unit 2270 periodically collects information such as a favorite place, a favorite sport, and a favorite hobby of the user 10 from the external data by using ChatGPT Plugins.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include determining a schedule of the user, and
in a case in which it is determined to propose a schedule as the action of the electronic apparatus, the action determination unit determines a schedule of the user to be proposed by using a sentence generation model on the basis of event data stored in the history data.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Fifth Embodiment

In autonomous processing in the present embodiment, the agent spontaneously collects all pieces of information related to the user. For example, in a case in which the user is at home, the agent ascertains when and what kind of question the user asks the agent, and when and what kind of action the user takes (for example, wakes up at 7:00 AM, turns on the television, checks the weather with the smartphone, and checks the train time with the train line information around 8:00). Since the agent spontaneously collects various types of information related to the user, even if the content of the question is unclear only when the user says “train” around 8:00 AM, the agent automatically converts the question into a correct question according to the need analysis that can be found from the word or the expression.

For example, in a case in which the agent is set in a mall such as Ion Mall (registered trademark), the agent ascertains when and what kind of question the user asks the agent. For example, the agent ascertains that a large number of users ask questions about where an umbrella section is in the evening and in the rainy time. Only when another user says “umbrella”, the content of the question is ascertained and a solution is presented, thereby realizing a conversion from merely “answering” reception to “conversation” with consideration. In this autonomous processing, surrounding information of the installation place of the agent is input, and an answer corresponding to the place is created. It is checked whether the question has been resolved for the question partner, and the correctness/incorrectness of the inquired question and answer is fed back, thereby permanently increasing the solution rate.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The user's speech is converted into a question and answered.

The robot action is “(11) The user's speech is converted into a question and answered.”, that is, even if the content of the question in the user's statement is unclear, the action determination unit 2236 automatically converts the question into a correct question and presents a solution.

Regarding “(11) The user's speech is converted into a question and answered.”, the storage control unit 2238 periodically detects an action of the user as the state of the user, and stores the detected action in the history data 2222 over time. The storage control unit 2238 may store surrounding information of the installation place of the agent in the history data 2222.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include autonomously converting a speech of the user into a question, and
the action determination unit answers the question as the action of the electronic apparatus.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Sixth Embodiment

In autonomous processing in the present embodiment, the robot 100 as an agent spontaneously collects various types of information from an information source such as a television or a web even when the user is absent. For example, in a case in which the robot 100 is still a child, that is, the robot 100 is still in the beginning of activation as an example, the robot 100 can hardly have a conversation. However, since the robot 100 always obtains various types of information when the user is absent, the robot 100 can learn and grow by itself. Therefore, the robot 100 gradually speaks human words. As an example, the robot 100 first generates animal words (sounds), but in the case of exceeding a certain condition, the robot learns human words and starts to speak human words.

In a case in which the user raises the robot 100 that can obtain a game feeling as if a pet talking to the user comes to the user's house, the robot 100 spontaneously learns and gradually memorizes words even when the user is absent. For example, in a case in which the user comes home from school, the robot 100 itself outputs a conversation “I memorized 10 words today, apple, koala, egg . . . ” to the user, so that a more realistic game of raising the robot 100 is realized.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (12).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot increases its vocabulary.
(12) The robot speaks the increased vocabulary.

In a case in which the action determination unit 2236 determines that, as the robot action, “(11) The robot increases its vocabulary.”, that is, the robot will increase its vocabulary, the robot 100 increases the vocabulary by itself and gradually learns human words even when the user is absent.

Regarding “(11) The robot increases its vocabulary.”, the related information collection unit 2270 spontaneously collects various types of information including vocabulary by accessing information sources such as a television and a web by itself even when the user is absent. Regarding “(11) The robot increases its vocabulary.”, the storage control unit 2238 stores various vocabularies on the basis of the information collected by the related information collection unit 2270.

In the present embodiment, in a case in which the action determination unit 2236 determines that “(11) The robot increases its vocabulary.” as the robot action, the robot 100 evolves spoken words by increasing its own vocabulary even when the user is absent. That is, the vocabulary of the robot 100 is improved. Specifically, initially, the robot 100 generates animal words (sounds), but gradually evolves and speaks human words according to the number of vocabularies collected by the robot 100 itself. As an example, for example, the level from animal words to human adult words is associated with a cumulative value of the number of vocabularies, and the robot 100 speaks words of age corresponding to the cumulative value by itself.

For example, in a case in which the robot 100 first generates a dog's sound, the robot can evolve the dog's sound into a human word according to the cumulative value of the stored vocabularies, and can finally speak human words. As a result, the user 10 can feel the process in which the robot 100 self-evolves from a dog to a human, that is, self-grows. In a case in which the robot 100 speaks human words, the user 10 can obtain a feeling as if a pet talking to the user comes to the user's house.

The initial sound emitted by the robot 100 enables the user 10 to set a favorite animal of the user 10, such as a dog, a cat, and a bear. The animal set in the robot 100 can be changed at a desired level. In a case in which the animal is reset, the words spoken by the robot 100 can be reset at the initial stage, or the level at which the animal is reset can be maintained.

In a case in which the action determination unit 2236 determines that, as the robot action, “(12) The robot speaks the increased vocabulary.”, that is, the robot will speak the increased vocabulary, the robot 100 speaks the vocabulary collected and increased by itself. Specifically, the vocabulary collected by the robot by itself is spoken to the user from when the user is absent until the user returns home or comes back. As an example, in the robot 100, for example, the robot 100 itself outputs a conversation “I memorized 10 words today, apple, koala, egg . . . ” to the user who has come home or come back.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, and the emotion of the electronic apparatus, and an action determination model at a predetermined timing, any of a plurality of types of device operations including non-operation as an action of the electronic apparatus, in which
the device operations include increasing a vocabulary and speaking the increased vocabulary, and
in a case in which it is determined to increase a vocabulary as the action of the electronic apparatus, the action determination unit increases the vocabulary, and in a case in which it is determined to speak the increased vocabulary, the action determination unit speaks the increased vocabulary.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Seventh Embodiment

In autonomous processing in the present embodiment, from all the actions of the user detected and stored by a pressure sensor (air pressure sensor) set in the agent's hand, a touch sensor set in the agent's nose, and the like, in a case in which an emotion value of the user at the time of detecting the user's gesture exceeds a certain value, the gesture is stored as a particularly important gesture. In a case in which the same gesture and emotion value of the user as before are detected at different timings, a speech such as “That emotion is the same as that emotion at that time. What's wrong?” can be performed spontaneously.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) A question about an important gesture is asked.

As the robot action, in a case in which “(11) A question about an important gesture is asked.”, that is, a gesture of the user coincides with the past important gesture, the action determination unit 2236 can spontaneously make a speech such as “You have the same emotion as that emotion at that time. What's wrong?”

Regarding “(11) A question about an important gesture is asked.”, the storage control unit 2238 stores the action of the user (gesture) in the history data 2222 together with the emotion value of the user. In a case in which the emotion value of the user exceeds a certain value, the action of the user is stored in the history data 222 as the important gesture. In the response processing, matching determination between the stored gesture of the user and the important gesture is performed.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include asking the user a question about an important action performed by the user in the past, and
the action determination unit stores, as the action of the electronic apparatus, the action of the user together with an emotion value of the user, stores the action as the important action in a case in which the emotion value of the user exceeds a predetermined value, and
determines to ask a question about the past important action in a case in which the action of the user at a different timing coincides with the important action.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

### Twenty-Eighth Embodiment

In autonomous processing in the present embodiment, the robot 100 spontaneously estimates the content of conversation between the robot and the user, for example, before the user speaks to the robot, also in consideration of a situation at that time, and the robot says what the user wants to say in advance or preemptively reproduces the user's favorite music. In order to execute such an action, the robot 100 tracks and stores characteristics of the user such as the user's personality, preference, habit, motion, idea, action, conversation content, and emotion as a history, and also stores a situation such as a temperature, a brightness, weather, a status, time, a season, and a place at the time of storing the characteristics of the user as a history.

The action determination unit 2236 uses at least one of the state of the user 10, the emotion of the user 10, the emotion of the robot 100, or the state of the robot 100, and the action determination model 2221 at a predetermined timing, to determine, as the action of the robot 100, any of a plurality of types of robot actions including no action. Here, a case in which a sentence generation model having an interaction function is used as the action determination model 2221 will be described as an example.

Specifically, the action determination unit 2236 inputs text representing at least one of the state of the user 10, the emotion of the user 10, the emotion of the robot 100, or the state of the robot 100 and text for inquiry about the robot actions to the sentence generation model, and determines an action of the robot 100 on the basis of an output of the sentence generation model.

For example, the plurality of types of robot actions include the following (1) to (11).
(1) The robot does nothing.
(2) The robot has a dream.
(3) The robot speaks to the user.
(4) The robot creates a picture diary.
(5) The robot proposes an activity.
(6) The robot proposes a person with whom the user should meet.
(7) The robot introduces news in which the user is interested.
(8) The robot edits pictures and moving images.
(9) The robot studies with the user.
(10) The robot evokes memory.
(11) The robot reproduces the user's favorite music.

In a case in which it is determined that “(3) The robot speaks to the user.”, that is, the robot 100 speaks to the user, as the robot action, the action determination unit 2236 determines the speech content of the robot corresponding to the user state and the emotion of the user or the emotion of the robot by using the sentence generation model. In this case, the action control unit 2250 causes a speaker included in the control target 2252 to output a voice representing the determined speech content of the robot. In a case in which the user 10 is absent around the robot 100, the action control unit 2250 stores the determined speech content of the robot in the action schedule data 2224 without outputting a voice representing the determined speech content of the robot.

Regarding “(3) The robot speaks to the user.”, the storage control unit 1238 may track the user's personality, preference, habit, motion, idea, action, conversation content, emotion, and the like, and store the tracking results in the history data as the characteristic information including the characteristics of the user. The storage control unit 2238 may store information regarding the situation such as a temperature, a brightness, weather, a status, time, a season, a place at the time of storing the above characteristic information in the history data as the situation information. This situation information may be stored in association with the characteristic information.

The action determination unit 2236 can estimate the content of a conversation between the robot 100 and the user 10 before the user 10 talks to the robot 100 by using the characteristic information and the situation information stored in the history data by the storage control unit 2238. In this case, in addition to the history data described above, the action determination unit 2236 can improve the estimation accuracy of the content of a conversation with the user by also using the situation information such as a temperature, a brightness, weather, a status, time, a season, a place at that time. As a result, the robot 100 can estimate the content about which the user trying to talk to the robot 100 wants to say and spontaneously speak the content, for example, before the user talks to the robot (for example, when the gaze of the user 10 is directed to the robot 100).

In order to acquire the above-described user characteristic information, the robot 100 may have various functions. Specifically, the robot 100 may include a camera function capable of imaging the face of the user 10, a microphone function capable of acquiring the voice of the user 10, a heat detection function capable of detecting the body temperature of the user 10, for example, thermography, and the like, as functions for acquiring the user's personality, motion, action, conversation content, emotion, and the like. The robot 100 may include a communication function capable of acquiring various types of information from the SNS of the user as a function for acquiring the user's preference, habit, idea, action, and the like. These functions can be realized by employing the sensor unit 2200, a temperature sensor (not illustrated), and the like. The characteristic information collected via the various functions described above may be stored in association with a specific user in the robot 100, a storage of the server 300, or the like.

In order to acquire the above-described situation information, the robot 100 may further include a temperature sensor, an illuminance sensor, a timer, and position detection means such as a GPS.

By the robot 100 performing the above-described speech, the user 10 does not need to ask the robot 100 a first question, and the user can recognize as if the robot 100 understood the user.

The robot 100 capable of such an interaction will function particularly effectively in a use application in which a plurality of interactions can be performed between the identified relatively small number of users 10 and the robot 100, for example, in an office or a care site.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, characteristic information including a characteristic of the user, and situation information at the time of acquiring the characteristic information, in which
the device operations include speaking to the user,
in a case in which it is determined to speak to the user as the action of the electronic apparatus, the action determination unit estimates interaction content of the user with the electronic apparatus on the basis of the history data and situation information at the time of speaking to the user, and determines speech content to the user on the basis of a result of the estimation.

### (Supplementary Note 2)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, characteristic information including a characteristic of the user, and situation information at the time of acquiring the characteristic information, in which
the device operations include reproducing specific music data, and
in a case in which it is determined to reproduce the specific music data as the action of the electronic apparatus, the action determination unit determines the specific music data to be reproduced on the basis of the history data and situation information at the time of reproducing the specific music data.

### (Supplementary Note 3)

The action control system according to Supplementary Note 1 or 2, in which the electronic apparatus executes the action of the electronic apparatus determined by the action determination unit on the user before the user speaks to the electronic apparatus.

### (Supplementary Note 4)

The action control system according to Supplementary Note 1 or 2, in which the electronic apparatus is a robot, and
the action determination unit determines any of a plurality of types of robot actions including no action as an action of the robot.

### (Supplementary Note 5)

The action control system according to Supplementary Note 4, in which the action determination model is a sentence generation model having an interaction function, and
the action determination unit inputs text representing at least one of the user state, a state of the robot, the emotion of the user, or an emotion of the robot and text for inquiry about the robot actions to the sentence generation model, and determines an action of the robot on the basis of an output of the sentence generation model.

### (Supplementary Note 6)

The action control system according to Supplementary Note 4, in which the robot is mounted on a stuffed toy or is connected to a control target device mounted on the stuffed toy in a wireless or wired manner.

### (Supplementary Note 7)

The action control system according to Supplementary Note 4, in which the robot is an agent for interacting with the user.

### Twenty-Ninth Embodiment

In autonomous processing in the present embodiment, the agent may spontaneously or periodically detect an action or a state of the user by monitoring the user. Specifically, the agent may monitor the user to track and analyze what kind of information the user is viewing on what website, that is, perform tracking of the user. The agent may be interpreted as an agent system that will be described later. Hereinafter, the agent system may be simply referred to as an agent.

The term “spontaneous” may be interpreted as the agent or the robot 100 by itself acquiring a state of the user without a trigger from the outside.

Examples of the trigger from the outside may include a question from the user to the robot 100 and an active action from the user to the robot 100. The term “periodic” may be interpreted as a specific cycle such as a unit of one second, a unit of one minute, a unit of one hour, a unit of several hours, a unit of several days, a unit of week, or a unit of day of the week.

The action of the user may be interpreted as the following action tendency of the user.
(1) The user stops by one or a plurality of specific stores in a commercial facility such as a department store in order for the user to purchase a specific product. The user is moving to a display area of a plurality of products in a specific store.
(2) In order for the user to purchase a specific product, the user views one or a plurality of specific electric commerce (EC) sites by using a smartphone or a personal computer.
(3) In order for the user to determine a specific travel destination or lodging destination, the user views information posted on one or a plurality of lodging reservation sites, travel sites, or the like by using a smartphone or a personal computer.
(4) In order for the user to purchase a specific financial product, the user views specific information posted on one or a plurality of financial information sites by using a smartphone, a personal computer, or the like.

Examples of the state of the user may include the following states of the user.
(1) A state in which the user continues to worry or think about which product to purchase while viewing the product in a specific store or repeating try-on
(2) A state in which the user continues to worry or think about which product to purchase while viewing products on one or a plurality of EC sites by using a smartphone or a personal computer
(3) A state in which the user continues to worry or think about which lodging, travel destination, or the like to use while viewing information posted on one or a plurality of lodging reservation sites, travel sites, or the like by using a smartphone or a personal computer
(4) A state in which the user continues to worry or think about which financial product to invest in while viewing information posted on one or a plurality of financial information sites by using a smartphone, a personal computer, or the like

In the autonomous processing, the agent may put a question to ChatGPT about the detected state or action of the user.

In the autonomous processing, an answer of ChatGPT to the question and action content for proposing a thing may be stored in association with each other. The action content may be interpreted as action content of the electronic apparatus that proposes at least one thing from two or more things. Specifically, the action content may be interpreted as action content of the electronic apparatus that proposes a specific thing on the basis of an answer of ChatGPT to the detected state or action of the user.

Information in which the answer to ChatGPT is associated with the action content proposing a thing may be recorded as table information in a storage medium such as a memory. The table information may be interpreted as specific information recorded in the storage unit.

In the autonomous processing, action content that proposes at least one thing from among two or more things with respect to the state or action of the user may be executed by using the specific information that is the stored table information. Specifically, in the autonomous processing, the state of the user may be detected spontaneously or periodically, and at least one thing may be proposed from among two or more things as the action of the electronic apparatus on the basis of the detected state or action of the user and the specific information.

The specific information may be interpreted as information answered by ChatGPT on the basis of at least one of history data regarding the user or information preferred by the user. That is, in the autonomous processing, at least one thing may be proposed from among two or more things as the action of the electronic apparatus on the basis of the detected state or action of the user and at least one of the history data regarding the user or the information preferred by the user.

Hereinafter, an example of action content that proposes a thing will be described.

For example, in a case in which the agent detects that the user cannot decide which one of the clothing manufactured by the company A and the clothing manufactured by the company B should be purchased by monitoring the operation content of the user who uses the smartphone, the agent itself puts a question to ChatGPT.

ChatGPT answers at least one of two or more things on the basis of at least one of the history data 2222 related to the user or the information preferred by the user.

The history data 2222 may include information obtained through tracking, for example, the user's personality, preference, habit, motion, idea, action, conversation content, and emotion.

The information preferred by the user may be interpreted as information included in the collected data 2223 described above. Specifically, the information preferred by the user may be interpreted as taste information representing a thing of interest of the user 10 stored in the collected data 2223. More specifically, the information preferred by the user may include information frequently searched or selected by the user, for example, fashion (trend) and world situation.

The information preferred by the user is not limited thereto, and may include information regarding society emitted from a plurality of information sources. The information regarding society may include at least one of news, economic situation, social situation, political situation, financial situation, international situation, sports news, entertainment news, birth and death news, cultural situation, or fashion.

For example, in response to a question “Which product should be proposed to the user who cannot decide which clothing to purchase?” of the agent, ChatGPT may answer “The company A product will be subject to price increase from April, so purchase of the company A product is recommended before price increase.” on the basis of at least one of the history data 222 regarding the user or the information preferred by the user.

ChatGPT may answer “The company B product will be subject to price reduction from April, so purchase of the company B product is recommended after price reduction.”.

ChatGPT may answer “In view of the tendency of the product that the user recently purchases, it is recommended to purchase the product of the company C that is more expensive than the products of the companies A and B but is similar to the products of the companies A and B.”.

The agent who has obtained the answer may propose at least one thing from two or more things on the basis of the detected state or action of the user and the recorded information. That is, the agent may refer to the recorded information and reproduce a voice corresponding to the content of the product suitable for the detected state or action of the user through a speaker mounted on the smartphone, the robot 100, or the like.

The agent may refer to the recorded information and display an image corresponding to the content of the product suitable for the detected state or action of the user on a screen mounted on the smartphone, the robot 100, or the like.

The agent may refer to the recorded information and display a message explaining the content of the product suitable for the detected state or action of the user on the screen mounted on the smartphone, the robot 100, or the like.

Instead of monitoring the operation content of the user who uses the smartphone, the agent may monitor the user who moves to a display location of a plurality of products in a specific store by using imaged data obtained by imaging the user with an imaging device.

As described above, according to the action control system of the disclosure, it is possible to determine action content of selecting at least one of two or more things and proposing the thing to the user by using at least one of the history data regarding the user or the information preferred by the user. Thus, for example, the agent spontaneously speaks to user who has difficulty in selecting a thing, so that a thing suitable for the user can be recommended and proposed.

The following supplementary notes regarding the above embodiment will be disclosed.

### (Supplementary Note 1)

An action control system including:
a state recognition unit that recognizes a user state including an action of a user and a state of an electronic apparatus;
an emotion determination unit that determines an emotion of the user or an emotion of the electronic apparatus;
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus; and
a storage control unit that stores, in history data, event data including an emotion value determined by the emotion determination unit and data including the action of the user, in which
the device operations include setting action content of selecting at least one of two or more things and proposing the thing to the user, and
in a case in which the action determination unit spontaneously or periodically detects a state of the user, and determines to propose at least one thing from among two or more things as the action of the electronic apparatus on the basis of at least one of the detected state of the user, history data regarding the user, or information preferred by the user, the action determination unit executes the action content.

### (Supplementary Note 2)

The action control system according to Supplementary Note 1, in which the electronic apparatus is a robot, and
the action determination unit determines any of a plurality of types of robot actions including no action as an action of the robot.

### (Supplementary Note 3)

The action control system according to Supplementary Note 2, in which the action determination model is a sentence generation model having an interaction function, and
the action determination unit inputs text representing at least one of the user state, a state of the robot, the emotion of the user, or an emotion of the robot, and text for inquiry about the robot actions to the sentence generation model, and determines an action of the robot on the

### (Supplementary Note 4)

The action control system according to Supplementary Note 2 or 3, in which the robot is mounted on a stuffed toy or is connected to a control target device mounted on the stuffed toy in a wireless or wired manner.

### (Supplementary Note 5)

The action control system according to Supplementary Note 2 or 3, in which the robot is an agent for interacting with the user.

The disclosure of Japanese Patent Application No. 2023-065099 filed on Apr. 12, 2023, the disclosure of Japanese Patent Application No. 2023-065715 filed on Apr. 13, 2023, the disclosure of Japanese Patent Application No. 2023-068063 filed on Apr. 18, 2023, the disclosure of Japanese Patent Application No. 2023-068818 filed on Apr. 19, 2023, the disclosure of Japanese Patent Application No. 2023-067491 filed on Apr. 17, 2023, the disclosure of Japanese Patent Application No. 2023-066668 filed on Apr. 14, 2023, the disclosure of Japanese Patent Application No. 2023-064494 filed on Apr. 11, 2023, the disclosure of Japanese Patent Application No. 2023-068070 filed on Apr. 18, 2023, the disclosure of Japanese Patent Application No. 2023-079537 filed on May 12, 2023, the disclosure of Japanese Patent Application No. 2023-079538 filed on May 12, 2023, the disclosure of Japanese Patent Application No. 2023-083516 filed on May 19, 2023, the disclosure of Japanese Patent Application No. 2023-083453 filed on May 19, 2023, the disclosure of Japanese Patent Application No. 2023-079540 filed on May 12, 2023, the disclosure of Japanese Patent Application No. 2023-083454 filed on May 19, 2023, the disclosure of Japanese Patent Application No. 2023-079541 filed on May 12, 2023, the disclosure of Japanese Patent Application No. 2023-078688 filed on May 11, 2023, the disclosure of Japanese Patent Application No. 2023-078689 filed on May 11, 2023, the disclosure of Japanese Patent Application No. 2023-078692 filed on May 11, 2023, the disclosure of Japanese Patent Application No. 2023-083426 filed on May 19, 2023, the disclosure of Japanese Patent Application No. 2023-080346 filed on May 15, 2023, the disclosure of Japanese Patent Application No. 2023-083455 filed on May 19, 2023, the disclosure of Japanese Patent Application No. 2023-080318 filed on May 15, 2023, the disclosure of Japanese Patent Application No. 2023-081011 filed on May 16, 2023, the disclosure of Japanese Patent Application No. 2023-080985 filed on May 16, 2023, the disclosure of Japanese Patent Application No. 2023-082447 filed on May 18, 2023, the disclosure of Japanese Patent Application No. 2023-083463 filed on May 19, 2023, the disclosure of Japanese Patent Application No. 2023-080389 filed on May 15, 2023, the disclosure of Japanese Patent Application No. 2023-081012 filed on May 16, 2023, the disclosure of Japanese Patent Application No. 2023-079676 filed on May 12, 2023, the disclosure of Japanese Patent Application No. 2023-079542 filed on May 12, 2023, the disclosure of Japanese Patent Application No. 2023-082448 filed on May 18, 2023, the disclosure of Japanese Patent Application No. 2023-079543 filed on May 12, 2023, and the disclosure of Japanese Patent Application No. 2023-082572 filed on May 18, 2023 are incorporated herein by reference in their entirety.

### REFERENCE SIGNS LIST

5 System
10, 11, 12 User
20 Communication network
100, 101, 102 Robot
100N Stuffed toy
200 Sensor unit
201 Microphone
202 Depth sensor
203 Camera
204 Distance sensor
210 Sensor module unit
211 Voice emotion recognition unit
212 Speech understanding unit
213 Expression recognition unit
214 Face recognition unit
220 Storage unit
221 Reaction rule
222 History data
230 User state recognition unit
232 Emotion determination unit
234 Action recognition unit
236 Action determination unit
238 Storage control unit
250 Action control unit
252 Control target
280 Communication processing unit
300 Server
500, 700 Agent system
1200 Computer
1210 Host controller
1212 CPU
1214 RAM
1216 Graphic controller
1218 Display device
1220 Input/output controller
1222 Communication interface
1224 Storage device
1226 DVD drive
1227 DVD-ROM
1230 ROM
1240 Input/output chip
2200 Sensor unit
2201 Microphone
2202 Depth sensor
2203 Camera
2204 Distance sensor
2210 Sensor module unit
2211 Voice emotion recognition unit
2212 Speech understanding unit
2213 Expression recognition unit
2214 Face recognition unit
2220 Storage unit
2221 Action determination model
2222 History data
2230 State recognition unit
2232 Emotion determination unit
2234 Action recognition unit
2236 Action determination unit
2238 Storage control unit
2250 Action control unit
2252 Control target
2270 Related information collection unit
2280 Communication processing unit

1. An action control system comprising:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content,
wherein the action determination unit reflects a detection result obtained by detecting a change in a body temperature of the user in generation of an answer of the interaction function, estimation of the emotion of the user, and estimation of the emotion of the robot.

2. An action control system comprising:
an emotion determination unit that determines an emotion of a user or an emotion of a robot; and
an action determination unit that generates action content of the robot with respect to an action of the user and the emotion of the user or the emotion of the robot on the basis of an interaction function of allowing the user and the robot to interact with each other, and determines an action of the robot corresponding to the action content,
wherein
the action determination unit determines the action by reflecting a detection result obtained by detecting a change in a body temperature of the user in generation of an answer of the interaction function, estimation of the emotion of the user, and estimation of the emotion of the robot, and
the action determined by the action determination unit includes an action of changing a surface temperature of at least a part of the robot.

3. (canceled)

4. (canceled)

5. (canceled)

6. (canceled)

7. (canceled)

8. (canceled)

9. (canceled)

10. An action control system comprising:
a state recognition unit that recognizes a user state including an action of a user and a state of an electronic apparatus;
an emotion determination unit that determines an emotion of the user or an emotion of the electronic apparatus; and
an action determination unit that determines, by using at least one of the user state, the state of the electronic apparatus, the emotion of the user, or the emotion of the electronic apparatus, and an action determination model at a predetermined timing, any of a plurality of types of device operations including non-operation as an action of the electronic apparatus,
wherein the action determination unit autonomously detects a body temperature of the user as a state of the user as the action of the electronic apparatus, and reflects the body temperature of the user in determination of the emotion of the user by the emotion determination unit on the basis of the body temperature of the user.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)
